(19) United States
(12) Patent Application Publication
(10) Pub. No.: US 2026/0041817 A1
(43) Pub. Date: Feb. 12, 2026

(54) BIODEGRADABLE TISSUE REPLACEMENT IMPLANT AND ITS USE

(71) Applicant: THE U.S.A., as represented by the Secretary, Department of Health and Human Services, Bethesda, MD (US)

(72) Inventors: Arvydas Maninishkis, Washington, DC (US); Kapil Bharti, Potomac, MD (US)

(73) Assignee: THE U.S.A., as represented by the Secretary, Department of Health and Human Services, Bethesda, MD (US)

(21) Appl. No.: 19/092,324

(22) Filed: Mar. 27, 2025

Related U.S. Application Data
(63) Continuation of application No. 17/295,030, now abandoned.
(60) Provisional application No. 62/769,484, filed on Nov. 19, 2018.

(51) Int. Cl. A61L 27/38; A61L 27/18; A61L 27/54; A61L 27/56

(52) U.S. Cl. CPC A61L 27/3813; A61L 27/18; A61L 27/3834; A61L 27/3869; A61L 27/54; A61L 27/56; A61L 2430/16

(57) ABSTRACT

Tissue replacement implants are disclosed that include polarized retinal pigment epithelial cells on a poly(lactic-co-glycolic acid) (PLGA) scaffold, wherein the PLGA scaffold is 20-30 microns in thickness, has a DL-lactide/glycotide ratio of about 1:1, an average pore size of less than about 1 micron, and a fiber diameter of about 150 to about 650 nm. Also disclosed are methods of treating a subject with a retinal degenerative disease, retinal or retinal pigment epithelium dysfunction, retinal degradation, retinal damage, or loss of retinal pigment epithelium. These methods include locally administering to the eye of the subject the tissue replacement implant. In further embodiments, methods are disclosed for producing the tissue replacement implant.

### CROSS REFERENCE TO RELATED APPLICATION

This is a continuation of U.S. patent Ser. No. 17/295,030, filed on May 18, 2021, which is a § 371 U.S. national stage of International Application No. PCT/US2019/062016, filed Nov. 18, 2019, which claims the benefit of U.S. Provisional Application No. 62/769,484, filed Nov. 19, 2018.

The prior applications are incorporated by reference herein.

### ACKNOWLEDGMENT OF GOVERNMENT SUPPORT

This invention was made with government support under project no. ZA1 #: ET000542-03 awarded by the National Institutes of Health, the National Eye Institute. The government has certain rights in the invention.

### REFERENCE TO AN ELECTRONIC SEQUENCE LISTING

The contents of the electronic sequence listing submitted herewith entitled sequence.xml having a size of 2,680 bytes, and a Date of Creation of Mar. 25, 2025, is herein incorporated by reference in its entirety.

### FIELD OF THE DISCLOSURE

This disclosure is related to the field of cell-based therapeutics, specifically to tissue replacement implants including retinal pigment epithelium (RPE) cells, and methods for producing and using these tissue replacement implants.

### BACKGROUND

Cell-based therapeutics offer the promise of ‘permanent’ replacement of degenerative tissue. The eye is an appealing area of interest due to its ease of accessibility and the urgent need for effective therapies to help a growing elderly population experiencing vision loss (Fischbach, et al., Sci Transl. Med 5, 179ps177 (2013); Song and Bharti, Brain Res 1638, 2-14 (2016)). The previous success with surgical procedures transplanting autologous RPE/choroid cells obtained from the periphery of the same patients' eyes has provided the critical proof-of-principle needed to develop pluripotent stem cell derived RPE based cell therapies (Joussen et al., Ophthalmology 114, 551-560 (2007); van Zeeburg, et al., Am J Ophthalmol 153, 120-127.e122 (2012)). In recent preliminary clinical studies, embryonic stem cell (ESC) and induced pluripotent stem cell (iPSC)-based therapies have been tested in patients with age-related macular degeneration (AMD), a leading cause of blindness among elderly (Schwartz et al., Lancet 379, 713-720 (2012); Schwartz et al., Lancet 385, 509-516 (2015); Mandai et al., N Engl J Med 376, 1038-1046 (2017); Bharti et al., Pigment Cell Melanoma Res 24, 21-34 (2011); da Cruz et al., Nat Biotechnol 36, 328-337 (2018); Kashani et al., Sci Transl Med 10, (2018)). AMD has two advanced stages: “dry” AMD or geographic atrophy is caused by the death of the retinal pigment epithelium (RPE), a monolayer of pigmented cells located in the back of the eye; the “wet” or choroidal neovascular AMD is caused by proliferation of choroidal vessels that penetrate through the RPE and leak fluid and blood under the retina (Ambati and Fowler, Neuron 75, 26-39 (2012); Bird, et al., JAMA Ophthalmol 132, 338-345 (2014)). Both conditions lead to photoreceptor cell death, causing serious vision loss and can lead to blindness.

To date, several studies have explored subretinal delivery of stem cell-derived RPE cells in AMD patients: an ESC-derived RPE cell suspension was tested in a phase I clinical trial of patients with geographic atrophy (“dry”) stage of AMD (Joussen et al., Ophthalmology 114, 551-560 (2007); van Zeeburg, et al., Am J Ophthalmol 153, 120-127.e122 (2012)); an autologous iPSC-RPE (iRPE) cells were recently transplanted in one patient with the “wet” form of AMD (Schwartz et al., Lancet 385, 509-516 (2015); Mandai et al., N Engl J Med 376, 1038-1046 (2017)). In addition, an ESC-derived RPE-on a paralene scaffold was tested in four “dry” AMD patients and the same cells on polyester scaffold was tested in two “wet” AMD patients (da Cruz et al., Nat Biotechnol 36, 328-337 (2018); Kashani et al., Sci Transl. Med 10 (2018)). These studies evidenced the safety of stem cell-based therapies, but they also unveiled the need for innovation to develop a commercially approved stem cell therapy to the clinic. For example, RPE cells in suspension do not self-organize into a confluent polarized monolayer and they do not provide barrier function in the back of patients' eyes, affecting the long-term survival of cells (Diniz et al., Investigative Ophthalmology & Visual Science 54, 5087-5096 (2013)). Previous approaches have not provided methods at were functionally validated in multiple patients (Kamao et al., Stem Cell Reports 2, 205-218 (2014)). Provided herein is a tissue replacement implant that is clinically effective for the treatment of a variety of retinal diseases and retinal dysfunction, and can be produced using Good Manufacturing Practice (GMP)/clinical-grade manufacturing.

### SUMMARY OF THE DISCLOSURE

In some embodiments, a tissue replacement implant is disclosed that includes polarized retinal pigment epithelial cells on a poly(lactic-co-glycolic acid) (PLGA) scaffold, wherein the PLGA scaffold is 20-30 microns in thickness, has a DL-lactide/glycotide ratio of about 1:1, an average size of pores in-between adjacent fibers of less than about 1 micron, and a fiber diameter of about 150 to about 650 nm.

In additional embodiments, disclosed are methods of treating a subject with a retinal degenerative disease, retinal or retinal pigment epithelium dysfunction, retinal degradation, retinal damage, or loss of retinal pigment epithelium. These methods include locally administering to the eye of the subject a disclosed tissue replacement implant.

In further embodiments, methods are disclosed for producing the tissue replacement implant. These methods include: a) obtaining PLGA coated with vitronectin, wherein the PLGA scaffold comprises fibers that forming mesh structure and wherein the PLGA scaffold has an upper surface and a lower surface, wherein the PLGA scaffold is about 20- about 30 microns in thickness, has a DL-lactide/glycotide ratio of about 1:1, an average pore size of less than about 1 microns, and a fiber diameter of about 150 to about 650 nm; b) treating the scaffold with heat to fuse fibers of the scaffold at the junctions of fiber intersections within the PLGA scaffold to increase mechanical strength of the PLGA scaffold & to reduce pore size; c) seeding retinal pigment epithelial cells onto the PLGA scaffold at about 125,000 to about 500,000 cells per 12 mm diameter of PLGA scaffold; and d) culturing the retinal pigment epithelial cells on the PLGA scaffold in a tissue culture medium in vitro, with medium present on both the upper surface and the lower surface of the PLGA scaffold, for a time that is sufficient for i) polarization of the retinal pigment epithelial cells and ii) bulk degradation of the PLGA in the scaffold.

The foregoing and other features and advantages of the invention will become more apparent from the following detailed description of a several embodiments, which proceeds with reference to the accompanying figures.

### BRIEF DESCRIPTION OF THE FIGURES

FIGS. 1A-1F. Clinical-grade 2D triphasic iPSC-RPE differentiation protocol generates pure RPE cells. (A) Workflow illustrating a pipeline to manufacture and test autologous clinical-grade iPSC-RPE-patches for filing an Investigational New Drug (IND)-application to the FDA for approval to start a phase I clinical trial. (B) Time-line of clinical-grade iRPE differentiation. Clinical-grade iRPE differentiation takes 77 days, is initiated with monolayer iPSCs and performed using xeno-free reagents. Neuro Ectoderm Induction Medium (NEIM); RPE Induction Medium (RPEIM); RPE Commitment Medium (RPECM); RPE Growth Medium (RPEGM); RPE Maturation Medium (RPEMM). (C) Coding-region sequencing of 223 oncogenes at 2000× depth was performed for all nine clinical-grade AMD iPSC clones. Kinship analysis of sequence confirms identity between iPSC clones and their respective donors, except for Donor 2 clone B that shows several sequence changes. (D, E) Flow cytometry analysis of clinical-grade iPSC-RPE derived from all three AMD patients, performed at the RPE progenitor stage day (D)17, RPE-commitment stage (D27), and immature RPE-stage (D42) reveals high PAX6/MITF double+ cells at the progenitor stage that progressively decline in number as cells mature towards RPE lineage (trendline). Consistently MITF+ cells increase in number from RPE progenitor to immature RPE stage. Furthermore, RPE-progenitor marker PMEL17+ and pigmentation enzyme TYRP1+ cells are high at the RPE-progenitor stage, whereas mature RPE-marker CRABP and BEST1+ cells start at the RPE-commitment stage and continue to increase to the immature RPE stage (trendline) (n=6). (F) Progressive increase in RPE-specific gene expression from D5-D42 of clinical-grade iPSC-RPE differentiation (n=6).

FIGS. 2A-2I. Biodegradable PLGA-scaffolds help generate functionally mature AMD iRPE-patches. (A) Young's Modulus of different PLGA-scaffolds is determined to identify the optimal scaffold for transplantation (**p<0.001). (B) SEM confirms surface topology of single-layer fused PLGA scaffold. (C) Maturation of iRPE-patch confirmed by immunostaining for mature-RPE marker RPE65 (cytoplasmic) and human-specific antigen STEM121 (cytoplasmic), top panel; RPE-pigmentation protein GPNMB (cytoplasmic) and Bruch's membrane protein COLLAGEN IV (basal), middle panel; and COLLAGEN VIII, Bruch's membrane marker (basal), bottom panel (n=3). (D) TEM of iRPE-patch on transwell membrane or the PLGA-scaffold confirms similar maturation. Basal infoldings are present only in the case of PLGA scaffold (inset) (n=3). (E) ΔcT values of RPE-specific genes are displayed for all eight iRPE-patches from three AMD donors (n=8). (F) Live TER measurement during the last three weeks (D54-77) of iRPE-patch maturation shows a progressive increase in patch electrical intactness and maturity. Representative data from three clones (3A, 3D, 4A) is displayed (n=8). (G) Graphs shows similar rate of phagocytosis for most AMD-iRPE-patches (n=8). (H, I) Principle Component Analysis (PCA) combining data from all assays (morphometric, gene expression, TER, and phagocytosis) shows most variation between clones across PC1, except for sample D2B (H). PCA plotted without D2B data revealed higher donor-to-donor variation as compared to clone-to-clone variation (I) (n=8).

FIGS. 3A-3K. Clinical-grade AMD iRPE-patch shows improved integration compared to injected RPE cells in rodent models. (A-C) En face infrared image (A), OCT (B), and immunohistochemistry for human antigen (STEM121—cytoplasmic, C) confirm the correct sub-retinal location and complete integration of the 0.5 mm diameter clinical-grade AMD-iRPE-patch (lighter arrowhead; darker arrowhead marks rat RPE cells—see inset for higher magnification) in the sub-retinal space of immunocompromised rat at ten weeks post-surgery. (n=20) (D) Immunohistochemistry for STEM121-(arrowhead, see inset for higher magnifications) confirms the presence of clinical-grade AMD-iRPE cells injected in rat eye. Rat RPE are not positive for STEM121 (arrowhead in the inset shows higher magnification). (E) The absence of Ki67 immunostaining confirms lacking proliferation in injected human cells (lighter arrowhead, see inset for higher magnification; rat RPE is marked with darker arrowhead). (F) STEM121—immunostaining (lighter arrowhead) also reveals integration of a small number of human cells in the rat RPE (darker arrowhead; see inset for higher magnification) (n=10). (G, I) Photomontage of RCS rat retina shows outer nuclear layer (ONL) is rescued (arrowheads) with transplanted iPSC-RPE-patch (˜2,500 cells on a 0.5 mm diameter patch) (G) or iRPE cell suspension (100,000 cells) (I), compared with the degenerated ONL in non-transplanted areas (arrow, n=10). (H, J) Immunofluorescence staining of iRPE-patch (H) or iRPE cell suspension (J) implanted retina with ONL rescue (light arrowheads) (HuNu, human nuclear antigen—nuclear, human-specific anti-PMEL17—cytoplasmic). Note, darker arrowhead in (H) points to iRPE cells that likely dislodged from the scaffold during transplantation. (K) Optokinetic tracking thresholds at P90. Even with a 40 times smaller dose, iRPE-patch performs similar to cell suspension at p90, suggesting the better efficacy of iRPE-patch (n=10, *p<0.05, **p<0.001).

FIGS. 4A-4I. Development of an iRPE-patch efficacy model by laser-induced RPE ablation in pigs. (A) Schematics of micropulse laser injuring the pig RPE; insert, fluorescein angiogram depicting laser-induced outer blood-retinal-barrier breakdown. (B, C) OCT reveals RPE detachment at 24 h post laser and RPE-thinning (arrowheads) 48h post 1% laser duty cycle. (D) Heatmap of the P1 values of the visual streak region after 1% or 3% duty cycle laser (laser areas outlined with dashed lines in). P1 value scale bar is indicated. (E) Average mfERG waveform from healthy, 1% and 3% duty cycle laser areas. (F-I) immunostaining for TUNEL (nuclear), RPE65 (cytoplasmic in RPE), and PNA (cytoplasmic in photoreceptors) (F, G) and H&E staining (H, I) at 48 hours reveal damaged, detached, and apoptotic RPE monolayer (arrowheads) with some damage to the ONL layer.

FIGS. 5A-5N. Clinical-grade AMD iRPE-patch is efficacious in a laser-induced retinal degeneration pig model. (A-C) Comparison of OCT from retina over a healthy region, retina transplanted with an empty PLGA scaffold, or a retina transplanted with clinical-grade AMD iRPE-patch (horizontal lines) shows integration of iRPE-patch and healthy retina over the patch as compared to empty scaffold where retinal tubulations can be seen. (D-F) Immunostaining for STEM 121 (cytoplasmic—RPE, arrowhead, F) and RPE65 (cytoplasmic—RPE) confirms integration of clinical-grade AMD iRPE-patch in the pig eye. PNA staining (bright signal in photoreceptor outer segments) shows better preservation of photoreceptors in iRPE-patch transplanted retina (F) as compared to empty scaffold transplanted retina (E, arrowhead marks retinal tubulations). (G) Immunostaining for (cone opsins is marked by arrowhead) confirms preservation of cone photoreceptors above the area of human (STEM121, cytoplasmic in the RPE) iRPE-patch transplantation. (H, I) Rhodopsin immunostaining shows phagocytosed (bright signal marked by arrowheads) photoreceptor outer segments (POS) by healthy pig RPE immunostained with RPE65 (cytoplasmic in the RPE) and by human iRPE cells immunostained with STEM121 (cytoplasmic in the RPE). Z-sections show POS localized inside pig and human RPE cells. (J-L) Heat maps of N1P1 mfERG responses show dampened signals in the visual streak area after laser ablation of the RPE (compare J to K), and partial signal recovery after transplantation of clinical-grade iRPE-patch (L). (M, N) Average mfERG waveform (M), and combined data (N), show a significantly improved (p<0.05) signal in clinical-grade iRPE-patch on PLGA (top lines) as compared to empty scaffold (bottom lines) over 10 weeks of follow up. n=7.

FIGS. 6A-6N. iRPE-patch shows better integration and retina recovery as compared to iRPE-cell suspension in a laser-induced retinal degeneration pig model. (A-H) OCT shows improved retina health in PLGA-iRPE-patch and transwell-iRPE-patch as compared to empty PLGA-scaffolds and iRPE cell suspension at 2 and 5 weeks post-transplantation in pigs with laser-injured RPE (horizontal line and arrowheads point to the transplants). (I-L) Immunostaining for human antigen STEM121 (cytoplasmic signal in the RPE) and RPE65 (cytoplasmic signal in the RPE) confirms integration of human iRPE-patch in the pig eye (horizontal line and lighter arrowhead in J) with organized outer nuclear layer (PNA white, darker arrowhead, J) in healthy area and iRPE-patch transplanted area as compared to minimal PNA staining in the empty-scaffold area and cell suspension transplant, suggesting recovery of photoreceptors overlying the iRPE-patch. n=3. (M, N) Individual and average mfERG responses show improved retinal health in transwell-iRPE-patch and PLGA-iRPE-patch as compared to the empty scaffold and iRPE cell injection (n=3; *p<0.05).

FIGS. 7A-7O. (A) Time-line for research-grade iPSC-RPE differentiation. Differentiation into mature RPE takes 107 days and is initiated using 3D iPSC aggregates in NeuroEctoderm Induction Medium (NEIM). (B, C) Removal of FGF2 from RPE Induction Medium (RPEIM) doubles the number of GFP-positive RPE cells, measured using a reporter iPSC line expressing GFP under the control of TYROSINASE gene promoter42. (D) MEK inhibition by PD0325901 in RPEIM reduced variability of iPSC differentiation into GFP-positive RPE progenitors. (E) Mild DUAL SMAD-inhibition with low amounts of NOGGIN (50 ng/ml) in RPEIM combined with ACTIVIN A (100 ng/ml) in RPE Commitment Medium (RPECM) further increases the number of GFP-positive RPE progenitors. (F) ACTIVIN A and WNT3a in RPECM do not show any synergistic increase in number of GFP-positive RPE progenitors, as shown recently15 (n=3). (G) Research-grade 3D iPSC aggregate-based differentiation protocol reproducibly generates RPE from multiple healthy and patient iPSC lines (fibroblasts or blood derived) (n=4). (H, I) Flow cytometry analysis of PAX6 and MITF positive RPE cells at D42 of research-grade differentiation protocol. (J) Confirmation of the epithelial phenotype in cells on day12 of clinical-grade differentiation protocol. (K, L) How cytometry analysis of OCT4 and TRA1-81 at D42 of clinical-grade iRPE differentiation protocol confirms the absence of iPSCs. (M-O) Three additional users (first user data in FIG. 1D) reproduced the clinical-grade iRPE differentiation protocol, shown by flow cytometry analysis of PAX6 and MITF double positive cells at D17.

FIGS. 8A-8H. (A-H) SEM images of PLGA scaffold from an edge (A-D) and en face (E-H) views during degradation, showing changes in surface topology and thinning of the entire scaffold due to bulk degradation of PLGA fibers (n=3).

FIGS. 9A-9J. (A, B) SEM confirms the presence of comparable apical processes on PLGA-iRPE-patch and transwell-iRPE-patch (n=3). (C, D) Electrophysiological traces confirm similar electrical properties of the PLGA-iRPE-patch and transwell-iRPE-patch (n=3). (E) Comparison of fold change in RPE-specific genes between PLGA-iRPE-patch and transwell-iRPE-patch as compared to iPSCs showed similar level of monolayer maturity on the two substrates. Note, however, that different biological replicates show less variable on PLGA scaffold as compared to the transwell (n=3). (F, G) Bright field and ZO-1 immunostained images of iRPE from AMD donor 4, iPSC clone C (D4C). (H) ZO-1 immunostained images are segmented using a convolutional neural network to highlight cell borders. Segmented images were used for morphometric analysis (Schaub et al, under preparation). (I) Morphometric analysis performed on ZO-1 stained images of iRPE-patches from all three AMD donors using REShAPE software. 75,000-100,000 cells are imaged per patch and images analyzed to determine the hexagonality score (how hexagonal is the cell) of each RPE cell. Data is displayed as violin plots with the center dot representing the mean and horizontal lines in each plot representing 99% data points. RPE-patch derived from different clones are shows at different dots (n=8). (J) Graph shows the polarized VEGF secretion (basal/apical ratio) for different AMD-iRPE-patch (n=8).

FIGS. 10A-10B. (A, B) In-vitro spiking studies performed by seeding mixed cultures of iPSC and RPE on scaffolds (100% iPSC; 10% iPSC+90% RPE; 1% iPSC+99% RPE; and 100% RPE). iPSC-markers (OCT4 and TRA 1-81) were evaluated at days 0, 2, 7, and 14 post seeding by flow cytometry (A). Gene expression analysis of iPSC (ZFP42, OCT4, NANOG, LIN28A, LEFTY1, DNMT38) and lineage-specific markers (mesoderm—VWF, S100A4, KDR; endoderm—GATA6, FOXA2, AFP; non-RPE ectoderm—SOX10, MAP2, GFAP) evaluated at days 0, 7 and 14 days post-seeding of mixed iPSC, RPE cultures. Data is displayed as heat maps of relative ΔcT values. All values are normalized to day 0 data (B). This experiment confirms that PLGA scaffolds and RPE maturation medium do not support iPSC or non-RPE lineage growth. Also, non-RPE lineages genes are not expressed in iRPE-cells.

FIGS. 11A-11F. (A) Schematic of 0.5 mm diameter iRPE-patch transplantation in rat sub-retinal space. Surgery starts with a 1.2 mm sclerotomy, followed by vitreous displacement with hyaluronic acid (HA), retinal detachment by HA injection in the sub-retinal space, iRPE-patch loading in the transplantation tool, delivery of the patch in the sub-retinal space, and flattening of retinal detachment by hyaluronic acid. (B) Visualization of successfully transplanted iRPE-patch in the sub-retinal space of a rat eye. (C) An Optical Coherence Tomography image confirming successful delivery of transplanted iRPE-patch in the sub-retinal space of a rat eye. (D-F) Immunohistochemistry for human-specific PMEL17 (arrowhead, D) confirms RPE phenotype of injected AMD iRPE cells suspension in the sub-retinal space at 10 weeks post-injection. In contrast, injected human iPSC suspension (STEM121—E) leads to teratoma formation (F) n=10

FIGS. 12A-12G. (A) heatmap showing the average mfERG electrical waveforms responses of the pig eye before the laser was performed. Laser ablation of RPE is performed in the highest electrical activity area, the visual streak (dotted line) of the pig eye. (B, C) OCT of 3% laser duty cycle at 24 and 48 hours post-laser shows RPE and ONL damage and sub-retinal edema (arrowhead). (D) 3% duty cycle laser area staining with TUNEL (nuclear signal) shows several apoptotic cells in the ONL at 24h. Line shows the RPE monolayer. (E) At 48h, with 3% duty cycle laser, both ONL and RPE look severely damaged, with several apoptotic cells (nuclear signal) and weak PNA (photoreceptor outer segment cytoplasmic signal) and RPE65 (cytoplasmic signal in the RPE) staining. (F, G) H &E staining of the 1% (F) and 3% (G) duty cycles laser after 48 hours. Healthy area is evident to the left in the 1% duty cycle (F, arrowhead). Note significantly disrupted photoreceptors in 3% laser as compared to 1% (arrowheads).

FIGS. 13A-13M. (A, B) Image of transplantation tool and the tool tip loaded with an empty-scaffold (arrowhead). (C-F) Schematic of the pig surgery. After a standard four port-vitrectomy, retina is detached using a 38G blunt tip cannula (C), followed by retinotomy (D), enlarging of the sclerotomy (E), and delivery of the human RPE-patch in the sub-retinal space (F). (G-I) Intra-operative fundus imaging and optical coherence tomography (iOCT) performed during surgery confirm delivery of the 4×2 mm scaffold at the intended sub-retinal location. (J, K) OCT of pig eyes two (J) and ten weeks (K) post-surgery confirm empty scaffold degradation in non-immunosuppressed pigs and no inflammation caused by degrading PLGA byproducts. (L) Post-surgery confirmation of empty scaffold degradation in non-immunosuppressed pigs and no inflammation caused by degrading PLGA byproducts. (M) N1P1 mfERG signal over the empty-scaffold shows a significant reduction until week 3, caused likely by the surgical procedure. The signal recovers over time as the surgical damage heals and the scaffold degrades (2-way ANOVA, p<0.0001) n=3.

FIGS. 14A-14I. (A) GFP expressing human iRPE-patch. (B, C) Fundus autofluorescence images showing GFP-positive 4×2 mm iRPE-patch under the retina 2 weeks (white arrowhead) and 10 weeks post-transplantation (white arrowhead shows the iRPE-patch and grey arrowhead shows the laser area). Note, human cells do not migrate away from the patch. (D-F) Images of human iRPE-patch transplanted area of pig retina stained for photoreceptors (PNA, white, D); and immunostained for human iRPE cells (STEM121, cytoplasmic, E) and RPE (RPE65, cytoplasmic, F). (G) Quantification of the number of nuclei in the outer nuclear layer (ONL) of pig retina above the area of empty scaffold transplantation as compared to the area of iRPE-patch transplantation. Numbers are presented as fraction of healthy retina. (H) Immunostaining for human nuclear antigen STEM101 (nuclear) and RPE65 (cytoplamic) confirms integration of human iRPE-patch in the pig eye. (I) 3D reconstruction of Rhodopsin (photoreceptor outer segments) and STEM121 (cytoplasmic in the RPE) (arrowheads) immunostained sections of human iRPE-patch transplanted in a pig eye shows photoreceptor outer segments (POS) inside human RPE cells.

FIGS. 15A-15L. (A-I) Fluorescein angiography (A, D, G) confirming micropulse laser based RPE-injury in pig eyes used for empty scaffold (A), transwell-iRPE-patch (D), and iRPE cell injection (G) transplantation. Intra-operative fundus imaging (B, E, H), and intra-operative OCT (C, F, I) confirming correct delivery of empty scaffold (B, C), transwell-iRPE-patch (E, F), and iRPE cell injection (H, I) (white arrowheads). (J-L) Images of human iRPE-patch transplanted area of pig retina stained for photoreceptors (PNA, cytoplasmic in photoreceptor outer segments, J); and immunostained for human iRPE cells (STEM121, cytoplasmic in the RPE, K) and RPE (RPE65, cytoplasmic in the RPE, L). Note, STEM121 label for human cells (see underlined in K) stops where the pig RPE begins.

FIG. 16. CRISPR mediated gene correction in Albinism iPSC. SEQ ID NO: 1, wherein X is C or T, is shown in the top panel, and SEQ ID NO: 1, wherein X is C, is shown in the bottom panel (see also SEQ ID NO: 2).

FIG. 17. RPE patch from patient cells and CRISPR-corrected iPSC.

FIG. 18. Histology of OCA2 patient RPE cells and CRISPR-corrected OCA2 RPE cells.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:
SEQ ID NOs: 1 and 2 are the nucleic acid sequence of a portion of the OCA2 gene.

### DETAILED DESCRIPTION OF SEVERAL EMBODIMENTS

The retina is a layer of specialized light sensitive neural tissue located at the inner surface of the eye of vertebrates. Light reaching the retina after passing the cornea, the lens and the vitreous humor is transformed into chemical and electrical events that trigger nerve impulses. The cells that are responsible for transduction, the process for converting light into these biological processes are specialized neurons called photoreceptor cells.

The retinal pigment epithelium (RPE) is a polarized monolayer of densely packed hexagonal cells in the mammalian eye that separates the neural retina from the choroid. The cells in the RPE contain pigment granules and perform a crucial role in retinal physiology by forming a blood-retinal barrier and closely interacting with photoreceptors to maintain visual function by absorbing the light energy focused by the lens on the retina. These cells also transport ions, water, and metabolic end products from the subretinal space to the blood and take up nutrients such as glucose, retinol, and fatty acids from the blood and deliver these nutrients to photoreceptors.

RPE cells are also part of the visual cycle of retinal: Since photoreceptors are unable to reisomerize all-trans-retinal, which is formed after photon absorption, back into 11-cis-retinal, retinal is transported to the RPE where it is reisomerized to 11-cis-retinal and transported back to the photoreceptors.

Many ophthalmic diseases, such as (age-related) macular degeneration, macular dystrophies such as Stargardt's and Stargardt's-like disease, Best disease (vitelliform macular dystrophy), and adult vitelliform dystrophy or subtypes of retinitis pigmentosa, are associated with a degeneration or deterioration of the retina itself or of the RPE. It has been demonstrated in animal models that photoreceptor rescue and preservation of visual function could be achieved by subretinal transplantation of RPE cells (Coffey et al. Nat. Neurosci. 2002:5, 53-56; Lin et al. Curr. Eye Res. 1996:15, 1069-1077; Little et al. Invest. Ophthalmol. Vis. Sci. 1996:37, 204-211; Sauve et al. Neuroscience 2002:114, 389-401). In addition, there is a need for methods and implants that can be used to treat physical injury of the retina.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Activin: Members of the transforming growth factor beta (TGF-beta) superfamily which participate in regulation of several biological processes, including cell differentiation and proliferation. Activin A is a member of this family that mediates its biological effects through a complex of transmembrane receptor serine/threonine kinases, and binds to specific Activin A receptors. It is a dimer composed of two subunits. Activin A participates in regulation of stem cell maintenance, via SMAD-dependent activation transcription of marker of pluripotency like POU class 5 homeobox 1 (Oct-3/4), nanog, nodal, and nodal-signaling regulators, Left-right determination factor 1 and 2 (Lefty-B and Lefty-A). Activin A also stimulates transcription of several hormones such as Gonadotropin-releasing hormone. An exemplary sequence for Activin A is provided in GENBANK® Accession No. NM_002192.

Agent: Any protein, nucleic acid molecule (including chemically modified nucleic acids), compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject).

Agonist or Inducer: An agent that binds to a receptor of a cell or a ligand of such a receptor and triggers a response by that cell, often mimicking the action of a naturally occurring substance. In one embodiment, a Frizzled (Fzd) agonist binds to a Fzd receptor and potentiates or enhances the Wnt/beta-catenin signaling pathway.

Alter: A change in an effective amount of a substance or parameter of interest, such as a polynucleotide, polypeptide or a property of a cell. An alteration in polypeptide or polynucleotide or enzymatic activity can affect a physiological property of a cell, such as the differentiation, proliferation, or senescence of the cell. The amount of the substance can be changed by a difference in the amount of the substance produced, by a difference in the amount of the substance that has a desired function, or by a difference in the activation of the substance. The change can be an increase or a decrease. The alteration can be in vivo or in vitro. In several embodiments, altering is at least about a 50%, 60%, 70%, 80%, 90%, 95%, 96%, 97%, 98%, 99%, or 100% increase or decrease in the effective amount (level) of a substance, the proliferation and/or survival of a cells, or the activity of a protein, such as an enzyme.

Animal: Living multi-cellular vertebrate organisms, a category that includes, for example, mammals and birds. The term mammal includes both human and non-human mammals. Similarly, the term “subject” includes both human and veterinary subjects, for example, non-human primates, dogs, cats, horses, rabbits, pigs, mice, rats, and cows.

Antagonist or Inhibitor: An agent that blocks or dampens a biochemical or biological response when bound to a receptor or a ligand of the receptor. Antagonists mediate their effects through receptor interactions by preventing agonist-induced responses. In one embodiment, a Frizzled (Fzd) antagonist binds to a Fzd receptor or to a Fzd ligand (such as Wnt) and inhibits the Wnt/beta-catenin signaling pathway.

Antibody: A polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (VH) region and the variable light (VL) region. Together, the VH region and the VL region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, IL); Kuby, J., Immunology, 3^{rd} Ed., W. H. Freeman & Co., New York, 1997.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

CD34: A transmembrane phosphoglycoprotein protein encoded by the CD34 gene in humans and other mammalian species. CD34 was first described on hematopoietic stem cells. Cells expressing CD34 (CD34⁺ cell) are normally found in the umbilical cord and bone marrow as hematopoietic cells, or in mesenchymal stem cells, and endothelial progenitor cells, amongst other cells. CD34+ cells can be isolated from blood samples using immunomagnetic or immunofluorescent methods.

Cell: A structural and functional unit of an organism that can replicate independently, is enclosed by a membrane, and contains biomolecules and genetic material. Cells used herein may be naturally-occurring cells or artificially modified cells (e.g., fusion cells, genetically modified cells, etc.).

The term “cell population” refers to a group of cells, typically of a common type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and depleted of one or more cell types.

The term “stem cell” refers to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also produced from somatic cells by reprogramming them to a pluripotent state by the expression of certain transcription factors associated with pluripotency; these cells are called “induced pluripotent stem cells” or “iPSCs”.

Differentiation: Refers to the process whereby relatively unspecialized cells (such as embryonic stem cells or other stem cells) acquire specialized structural and/or functional features characteristic of mature cells. Similarly, “differentiate” refers to this process. Typically, during differentiation, cellular structure alters and tissue-specific proteins appear.

Defined or “Fully Defined:” In relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants and energy sources) which is supplemented with recombinant albumin, chemically defined lipids, and recombinant insulin. An exemplary fully defined medium is ESSENTIAL 8™ medium.

Embryoid Bodies: Three-dimensional aggregates of pluripotent stem cells. These cells can undergo differentiation into cells of the endoderm, mesoderm and ectoderm. In contrast to monolayer cultures, the spheroid structures that are formed when pluripotent stem cells aggregate enables the non-adherent culture of EBs in suspension, which is useful for bioprocessing approaches. The three-dimensional structure, including the establishment of complex cell adhesions and paracrine signaling within the EB microenvironment, enables differentiation and morphogenesis.

Embryo: A cellular mass obtained by one or more divisions of a zygote or an activated oocyte with an artificially reprogrammed nucleus without regard to whether it has been implanted into a female. A “morula” is the preimplantation embryo 3-4 days after fertilization, when it is a solid mass, generally composed of 12-32 cells (blastomeres). A “blastocyst” refers to a preimplantation embryo in placental mammals (about 3 days after fertilization in the mouse, about 5 days after fertilization in humans) of about 30-150 cells. The blastocyst stage follows the morula stage, and can be distinguished by its unique morphology. The blastocyst is generally a sphere made up of a layer of cells (the trophectoderm), a fluid-filled cavity (the blastocoel or blastocyst cavity), and a cluster of cells on the interior (the inner cell mass, ICM). The ICM, consisting of undifferentiated cells, gives rise to what will become the fetus if the blastocyst is implanted in a uterus.

Embryonic stem cells: Embryonic cells derived from the inner cell mass of blastocysts or morulae, optionally that have been serially passaged as cell lines. The term includes cells isolated from one or more blastomeres of an embryo, preferably without destroying the remainder of the embryo. The term also includes cells produced by somatic cell nuclear transfer. “Human embryonic stem cells” (hES cells) includes embryonic cells derived from the inner cell mass of human blastocysts or morulae, optionally that have been serially passaged as cell lines. The hES cells may be derived from fertilization of an egg cell with sperm or DNA, nuclear transfer, parthenogenesis, or by means to generate hES cells with homozygosity in the HLA region. Human ES cells can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. Human embryonic stem cells include, but are not limited to, MAO1, MAO9, ACT-4, No. 3, H1, H7, H9, H14 and ACT30 embryonic stem cells. Human embryonic stem cells, regardless of their source or the particular method use to produce them, can be identified based on (i) the ability to differentiate into cells of all three germ layers, (ii) expression of at least Oct-4 and alkaline phosphatase, and (iii) ability to produce teratomas when transplanted into immunocompromised animals.

Expand: A process by which the number or amount of cells in a cell culture is increased due to cell division. Similarly, the terms “expansion” or “expanded” refers to this process. The terms “proliferate,” “proliferation” or “proliferated” may be used interchangeably with the words “expand,” “expansion”, or “expanded.” Typically, during an expansion phase, the cells do not differentiate to form mature cells, but divide to form more cells.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

Feeder layer: Non-proliferating cells (such as irradiated cells) that can be used to support proliferation of stem cells. Protocols for the production of feeder layers are known in the art, and are available on the internet, such as at the National Stem Cell Resource website, which is maintained by the American Type Culture Collection (ATCC). “Feeder-free” or “feeder-independent” is used herein to refer to a culture supplemented with cytokines and growth factors (e.g., TGFβ, bFGF, LIF) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL®) or are grown on a substrate such as fibronectin, collagen or vitronectin. These approaches allow human stem cells to remain in an essentially undifferentiated state without the need for mouse fibroblast “feeder layers.”

Fetus: A developing mammal at an embryonic stage before birth. In humans, the fetal stage of prenatal development starts at the beginning of the 9th week after fertilization. In human eyes and RPE are already formed at 4 weeks from conception. RPE continues to mature for next several weeks.

Fibroblast growth factor (FGF): Any suitable fibroblast growth factor, derived from any animal, and functional fragments thereof, such as those that bind the receptor and induce biological effects related to activation of the receptor. A variety of FGFs are known and include, but are not limited to, FGF-1 (acidic fibroblast growth factor), FGF-2 (basic fibroblast growth factor, bFGF), FGF-3 (int-2), FGF-4 (hst/K-FGF), FGF-5, FGF-6, FGF-7, FGF-8, FGF-9 and FGF-98. “FGF” refers to a fibroblast growth factor protein such as FGF-1, FGF-2, FGF-4, FGF-6, FGF-8, FGF-9 or FGF-98, or a biologically active fragment or mutant thereof. The FGF can be from any animal species. In one embodiment, the FGF is mammalian FGF, including but not limited to, rodent, avian, canine, bovine, porcine, equine and human. The amino acid sequences and method for making many of the FGFs are well known in the art.

The amino acid sequence of human bFGF and methods for its recombinant expression are disclosed in U.S. Pat. No. 5,439,818, herein incorporated by reference. The amino acid sequence of bovine bFGF and various methods for its recombinant expression are disclosed in U.S. Pat. No. 5,155,214, also herein incorporated by reference. When the 146 residue forms are compared, their amino acid sequences are nearly identical, with only two residues that differ. Recombinant bFGF-2, and other FGFs, can be purified to pharmaceutical quality (98% or greater purity) using the techniques described in detail in U.S. Pat. No. 4,956,455.

An FGF inducer includes an active fragment of FGF. In its simplest form, the active fragment is made by the removal of the N-terminal methionine, using well-known techniques for N-terminal methionine removal, such as a treatment with a methionine aminopeptidase. A second desirable truncation includes an FGF without its leader sequence. Those skilled in the art recognize the leader sequence as the series of hydrophobic residues at the N-terminus of a protein that facilitate its passage through a cell membrane but that are not necessary for activity and that are not found on the mature protein. Human and murine bFGF are commercially available.

Frizzled (Fzd): A family of seven-pass transmembrane mammalian proteins that have characteristics of G-protein-coupled receptors and that bind proteins of the Wnt family of lipoglycoproteins, secreted Frizzled-related proteins (sFRPs), R-spondin, and Norrin and activates downstream signaling. Frizzled proteins (also referred to as Frizzled receptors) contain a cysteine-rich domain (CRD) that binds its cognate ligands, a carboxy terminal PDZ (Psd-95/disc large/ZO-1 homologous)-binding domain, and various consensus sites for serine/threonine kinases and tyrosine kinases. Amino acid hydropathy analysis indicates that the Frizzled proteins contain one extracellular amino terminus, three extracellular protein loops, three intracellular protein loops, and an intracellular carboxy terminus.

Frizzled proteins have an important regulatory role during embryonic development and have also been associated, in humans and in animal models, with a number of diseases, including various cancers, cardiac hypertrophy, familial exudative vitreoretinopathy, and schizophrenia.

There are at least 10 mammalian Frizzled proteins and the genes encoding the mammalian Frizzled proteins are related to the Drosophila frizzled genes. The human Frizzled proteins include Frizzled1 (Fzd1; GENBANK® Accession No. AB017363), Frizzled2 (Fzd2; GENBANK® Accession Nos. L37882/NM_001466), Frizzled3 (Fzd3; GENBANK® Accession No. AJ272427), Frizzled4 (Fzd4; GENBANK® Accession No. AB032417), Frizzled5 (Fzd5; GENBANK® Accession No. U43318), Frizzled6 (Fzd6; GENBANK® Accession No. AB012911), Frizzled7 (Fzd7; GENBANK® Accession No. AB010881), Frizzled8 (Fzd8; GENBANK® Accession No. AB043703), Frizzled9 (Fzd9; GENBANK® Accession Nos. U82169/NM_003508) and Frizzled10 (Fzd10; GENBANK® Accession No. AB027464). All of the GENBANK® entries are incorporated herein by reference as available on Jan. 1, 2013.

Growth factor: A substance that promotes cell growth, survival, and/or differentiation. Growth factors include molecules that function as growth stimulators (mitogens), factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are a fibroblast growth factor (such as FGF-2), epidermal growth factor (EGF), cilliary neurotrophic factor (CNTF), and nerve growth factor (NGF), and activin-A.

Haplotype: A combination of alleles at multiple loci along a single chromosome. A haplotype can be based upon a set of single-nucleotide polymorphisms (SNPs) on a single chromosome and/or the alleles in the major histocompatibility complex. The term “haplotype-matched” refers to a cell (e.g. iPSC) and the subject being treated share one or more major histocompatibility locus haplotypes. The haplotype of the subject can be readily determined using assays well known in the art. The haplotype-matched iPSC can be autologous or allogeneic. The autologous cells which are grown in tissue culture and differentiated to RPE cells inherently are haplotype-matched to the subject. “Substantially the same HLA type” indicates that the HLA type of donor matches with that of a patient to the extent that the transplanted cells, which have been obtained by inducing differentiation of iPSCs derived from the donor's somatic cells, can be engrafted when they are transplanted to the patient.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Isolated: An “isolated” biological component, such as a nucleic acid, protein or organelle that has been substantially separated or purified away from other biological components in the environment (such as a cell) in which the component naturally occurs, i.e., chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Similarly, an “isolated” cell has been substantially separated, produced apart from, or purified away from other cells of the organism in which the cell naturally occurs. Isolated cells can be, for example, at least 99%, at least 98%, at least 97%, at least 96%, 95%, at least 94%, at least 93%, at least 92%, or at least 90% pure.

Mammal: This term includes both human and non-human mammals. Examples of mammals include but are not limited to: humans and veterinary and laboratory animals, such as pigs, cows, goats, cats, dogs, rabbits and mice.

Marker or Label: An agent capable of detection, for example by ELISA, spectrophotometry, flow cytometry, immunohistochemistry, immunofluorescence, microscopy, Northern analysis or Southern analysis. For example, a marker can be attached to a nucleic acid molecule or protein, thereby permitting detection of the nucleic acid molecule or protein. Examples of markers include, but are not limited to, radioactive isotopes, nitorimidazoles, enzyme substrates, co-factors, ligands, chemiluminescent agents, fluorophores, haptens, enzymes, and combinations thereof. Methods for labeling and guidance in the choice of markers appropriate for various purposes are discussed for example in Sambrook et al. (Molecular Cloning: A Laboratory Manual, Cold Spring Harbor, New York, 1989) and Ausubel et al. (In Current Protocols in Molecular Biology, John Wiley & Sons, New York, 1998).

In some embodiments, the marker is a fluorophore (“fluorescent label”). Fluorophores are chemical compounds, which when excited by exposure to a particular wavelength of light, emits light (i.e., fluoresces), for example at a different wavelength. Fluorophores can be described in terms of their emission profile, or “color.” Green fluorophores, for example Cy3, FITC, and Oregon Green, are characterized by their emission at wavelengths generally in the range of 515-540. Red fluorophores, for example Texas Red, Cy5 and tetramethylrhodamine, are characterized by their emission at wavelengths generally in the range of 590-690. In other embodiments, the marker is a protein tag recognized by an antibody, for example a histidine (His)-tag, a hemagglutinin (HA)-tag, or a c-Myc-tag.

Membrane potential: The electrical potential of the interior of the cell with respect to the environment, such as an external bath solution. One of skill in the art can readily assess the membrane potential of a cell, such as by using conventional whole cell techniques. The membrane potential can be assessed using many approaches, such as using conventional whole cell access, or using, for example, perforated-patch whole-cell and cell-attached configurations.

Medium: A synthetic set of culture conditions with the nutrients necessary to support the growth (cell proliferation/expansion) and/or differentiation of a specific population of cells. In one embodiment, the cells are stem cells, such as iPSCs. In another embodiment, the cells are RPE cells. Media generally include a carbon source, a nitrogen source and a buffer to maintain pH. In one embodiment, growth medium contains a minimal essential media, such as DMEM, supplemented with various nutrients to enhance stem cell growth. Additionally, the minimal essential media may be supplemented with additives such as horse, calf or fetal bovine serum.

“Retinal Induction Medium (RIM)” refers to a growth media that comprises a WNT pathway inhibitor and a BMP pathway inhibitor and can result in the differentiation of PSCs to retinal lineage cells. The RIM also comprises a TGFβ pathway inhibitor.

“Retinal Differentiation Medium (RDM)” is a medium that comprises a WNT pathway inhibitor, a BMP pathway inhibitor and a MEK inhibitor and differentiates retinal cells. The RDM also comprises a TGFβ pathway inhibitor.

“Retinal Medium (RM)” refers to as a growth medium for the culture of retinal cells comprising Activin A and Nicotinamide.

“RPE-Maturation Medium (RPE-MM)” refers to a medium for the maturation of RPE cells comprising taurine and hydrocortisone. The RPE-MM also comprises triiodothyronine. The RPE-MM may also comprise PD0325901 or PGE2.

Nodal: A secretory protein encoded by the NODAL gene that belongs to the Transforming Growth Factor (TGF-beta) superfamily. During embryonic development, the left-right (LR) asymmetry of visceral organs in vertebrates is established through nodal signaling. Nodal is expressed in the left side of the organism in early development and it is highly conserved among deuterostomes. Exemplary Nodal sequences can be found as GENBANK® Accession Nos. NM_018055.4 and NP_060525.3, Jan. 6, 2013, which are incorporated by reference herein.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In some examples, a nucleic acid encodes a disclosed antigen.

Noggin: A protein which is encoded by the NOG gene. Noggin inhibits TGF-β signal transduction by binding to TGF-β family ligands and preventing them from binding to their corresponding receptors. Noggin plays a key role in neural induction by inhibiting BMP4, along with other TGF-β signaling inhibitors such as chordin and follistatin. Exemplary sequences for Noggin are GENBANK® Accession Nos. NP_005441.1 and NM_005450.4, Jan. 13, 2013, which are incorporated herein by reference.

Oct-4: A protein also known as POU5-F1 or MGC22487 or OCT3 or OCT4 or OTF3 or OTF4, that is the gene product of the Oct-4 gene. The term includes Oct-4 from any species or source and includes analogs and fragments or portions of Oct-4 that retain the ability to be used for the production of iPSCs. The Oct-4 protein may have any of the known published sequences for Oct-4 which can be obtained from public sources such as GENBANK®. An example of such a sequence includes, but is not limited to, GENBANK® Accession No. NM_002701.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: Conventional pharmaceutically acceptable carriers are useful for practicing the methods and forming the compositions disclosed herein. Remington's Pharmaceutical Sciences, by E. W. Martin, Mack Publishing Co., Easton, PA, 15th Edition, 1975, describes examples of compositions and formulations suitable for pharmaceutical delivery of the antimicrobial compounds herein disclosed.

In general, the nature of the carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pluripotent stem cells: Stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); and (c) express one or more markers of embryonic stem cells (e.g., express Oct 4, alkaline phosphatase, SSEA-3 surface antigen, SSEA-4 surface antigen, nanog, TRA-1-60, TRA-1-81, SOX2, REX1, etc), but that cannot form an embryo and the extraembryonic membranes (are not totipotent).

Exemplary pluripotent stem cells include embryonic stem cells derived from the inner cell mass (ICM) of blastocyst stage embryos, as well as embryonic stem cells derived from one or more blastomeres of a cleavage stage or morula stage embryo (optionally without destroying the remainder of the embryo). These embryonic stem cells can be generated from embryonic material produced by fertilization or by asexual means, including somatic cell nuclear transfer (SCNT), parthenogenesis, and androgenesis. PSCs alone cannot develop into a fetal or adult animal when transplanted in utero because they lack the potential to contribute to all extraembryonic tissue (e.g., placenta in vivo or trophoblast in vitro).

Pluripotent stem cells also include “induced pluripotent stem cells (iPSCs)” generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell. iPSCs are similar in properties to embryonic stem cells.

Polypeptide: A polymer in which the monomers are amino acid residues that are joined together through amide bonds. When the amino acids are alpha-amino acids, either the L-optical isomer or the D-optical isomer can be used, the L-isomers being preferred in nature. The term polypeptide is specifically intended to cover naturally occurring proteins, as well as those that are recombinantly or synthetically produced.

Substantially purified polypeptide as used herein refers to a polypeptide that is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Variant amino acid sequences may, for example, be 80, 90 or even 95 or 98% identical to the native amino acid sequence. Programs and algorithms for determining percentage identity can be found at the NCBI website.

Pre-confluent: A cell culture in which the proportion of the culture surface which is covered by cells is about 60-80%. In one embodiment, pre-confluent refers to a culture in which about 70% of the culture surface is covered by cells.

Prenatal: Existing or occurring before birth. Similarly, “postnatal” is existing or occurring after birth.

Recombinant: A recombinant nucleic acid or polypeptide molecule is one that has a sequence that is not naturally occurring or has a sequence that is made by an artificial combination of two otherwise separated segments of sequence. This artificial combination can be accomplished by chemical synthesis of polypeptide or nucleic acid molecules, or by the artificial manipulation of isolated segments of nucleic acid molecules, such as by genetic engineering techniques.

Retinal pigment epithelial (RPE) cell: RPE cells can be recognized based on pigmentation, epithelial morphology, and apical-basal polarized cells. RPE cells express, both at the mRNA and protein level, one or more of the following: Pax6, MITF, RPE65, CRALBP, PEDF, Bestrophin and/or Otx2. In certain other embodiments, the RPE cells express, both at the mRNA and protein level, one or more of Pax-6, MitF, and tyrosinase. RPE cells do not express (at any detectable level) the embryonic stem cell markers Oct-4, nanog, or Rex-1. Specifically, expression of these genes is approximately 100-1000 fold lower in RPE cells than in ES cells or iPSCs, when assessed by quantitative RT-PCR. Differentiated RPE cells also can be visually recognized by their cobblestone morphology and the initial appearance of pigment. In addition, differentiated RPE cells have trans epithelial resistance/TER, and trans epithelial potential/TEP across the monolayer (TER>100 ohms·cm²; TEP>2 mV), transport fluid and CO₂ from apical to basal side, and regulate a polarized secretion of cytokines.

“Mature” RPE cells are referred to herein as RPE cells which have downregulated expression of immature RPE markers such as Pax6 and upregulated expression of mature RPE markers such as RPE65.

RPE cell “maturation” refers herein to the process by which RPE developmental pathways are modulated to generate mature RPE cells. For example, modulation of cilia function can result in RPE maturation. “Retinal lineage cells” herein refer to cells that can give rise or differentiate to RPE cells.

Retinal Pigment Epithelium: The pigmented layer of hexagonal cells, present in vivo in mammals, just outside of the neurosensory retinal that is attached to the underlying choroid. These cells are densely packed with pigment granules, and shield the retinal from incoming light. The retinal pigment epithelium also serves as the limiting transport factor that maintains the retinal environment by supplying small molecules such as amino acid, ascorbic acid and D-glucose while remaining a tight barrier to choroidal blood borne substances.

Secreted Frizzled-related protein (sFRP): The sFRP family of proteins are approximately 32-40 kDa glycoproteins that were identified as antagonists of Wnt signaling (Rattner et al. (1997) Proc. Natl. Acad. Sci. USA 94:2859-63; Melkonyan et al. (1997) Proc. Natl. Acad. Sci. USA 94:13636-41; Finch et al. (1997) Proc. Natl. Acad. Sci. USA 94:6770-5; Uren et al. (2000) J. Biol. Chem. 275:4374-82; Kawano et al. (2003) J. Cell. Sci. 116:2627-34). In mammals, there are five sFRPs. The human sFRPs include sFRP1 (GENBANK® Accession No. AF001900.1), sFRP2 (GENBANK® Accession No. NM_003013.2), sFRP3 (GENBANK® Accession No. U91903.1), sFRP4 (GENBANK® Accession No. NM_003014.3), and sFRP5 (GENBANK® Accession No. NM_003015.3), as available on Jan. 1, 2013.

The sFRPs contain three structural units: an amino terminal signal peptide, a Frizzled type cysteine-rich domain (CRD), and a carboxy-terminal netrin (NTR) domain. The CRD spans approximately 120 amino acids, contains 10 conserved cysteine residues, and has 30-50% sequence similarity to the CRD of Fzd receptors. The netrin domain is defined by six cysteine residues and several conserved segments of hydrophobic residues and secondary structures. The biological activity of sFRPs is largely attributed to their role as regulators of Wnt function.

Sonic hedgehog (SHH): Sonic hedgehog (SHH) is one of three mammalian homologs of the Drosophila hedgehog signaling molecule and is expressed at high levels in the notochord and floor plate of developing embryos. SHH is known to play a key role in neuronal tube patterning (Echerlard et al., Cell 75:1417-30, 1993), the development of limbs, somites, lungs and skin. Moreover, overexpression of SHH has been found in basal cell carcinoma. Exemplary amino acid sequences of SHH is set forth in U.S. Pat. No. 6,277,820. An exemplary sequence for human Sonic is set forth as GENBANK Accession No. NG_007504.1 (Jan. 1, 2013), which is incorporated by reference herein.

Subject: An animal or human subjected to a treatment, observation or experiment.

Super donors: Individuals that are homozygous for certain MHC class I and II genes. These homozygous individuals can serve as super donors and their cells, including tissues and other materials comprising their cells, can be transplanted in individuals that are either homozygous or heterozygous for that haplotype. The super donor can be homozygous for the HLA-A, HLA-B, HLA-C, HLA-DR, HLA-DP or HLA-DQ locus/loci alleles, respectively.

Tissue Replacement Implant: A biological compatible structure including a both a matrix and cells that is created in vitro, that can be used to replace a tissue in vivo.

Totipotent or totipotency: A cell's ability to divide and ultimately produce an entire organism including all extraembryonic tissues in vivo. In one aspect, the term “totipotent” refers to the ability of the cell to progress through a series of divisions into a blastocyst in vitro. The blastocyst comprises an inner cell mass (ICM) and a trophectoderm. The cells found in the ICM give rise to pluripotent stem cells (PSCs) that possess the ability to proliferate indefinitely, or if properly induced, differentiate in all cell types contributing to an organism. Trophectoderm cells generate extraembryonic tissues, including placenta and amnion.

Treatment: Therapeutic measures that cure, slow down, lessen symptoms of, and/or halt progression of a diagnosed pathologic condition or disorder. In certain embodiments, treating a subject with a retinal disorder results in a decline in the deterioration of the retinal; an increase in the number of retinal pigment epithelial cells, an improvement in vision, or some combination of effects.

Tyrosinase: A copper-containing oxidase that catalyzes the production of melanin and other pigments from tyrosine by oxidation. This enzyme is the rate limiting enzyme for controlling the production of melanin. Tyrosinase acts in the hydroxylation of a monophenol and, the conversion of an o-diphenol to the corresponding o-quinone. o-Quinone undergoes several reactions to eventually form melanin. In humans, the tyrosinase enzyme is encoded by the TYR gene. Exemplary amino acid and nucleic acid sequences are set forth in GENBANK® Accession Nos. NM_000372.4 (human) and NM_011661.4 (mouse), Jan. 5, 2013, and which are incorporated by reference herein.

Undifferentiated: Cells that display characteristic markers and morphological characteristics of undifferentiated cells, distinguishing them from differentiated cells of embryo or adult origin. Thus, in some embodiments, undifferentiated cells do not express cell lineage specific markers, including, but no limited to, RPE markers.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. A vector may also include a sequence encoding for an amino acid motif that facilitates the isolation of the desired protein product such as a sequence encoding maltose binding protein, c-myc, or GST.

Wnt: A family of highly conserved secreted signaling molecules that regulate cell-to-cell interactions and are related to the Drosophila segment polarity gene, wingless. In humans, the Wnt family of genes encodes 38 to 43 kDa cysteine rich glycoproteins. The Wnt proteins have a hydrophobic signal sequence, a conserved asparagine-linked oligosaccharide consensus sequence (see e.g., Shimizu et al Cell Growth Differ 8:1349-1358 (1997)) and 22 conserved cysteine residues. Because of their ability to promote stabilization of cytoplasmic beta-catenin, Wnt proteins can act as transcriptional activators and inhibit apoptosis. Overexpression of particular Wnt proteins has been shown to be associated with certain cancers.

The Wnt family contains at least 19 mammalian members. Exemplary Wnt proteins include Wnt-1, Wnt-2, Wnt2b, Wnt-3, Wnt-3a, Wnt-4, Wnt-5a, Wnt5b, Wnt-6, Wnt-7a, Wnt-7b, Wnt-8a, Wnt-8b, Wnt9a, Wnt9b, Wnt10a, Wnt-10b, Wnt-11, and Wnt 16. These secreted ligands activate at least three different signaling pathways. In the canonical (or Wnt/beta-catenin) Wnt signaling pathway, Wnt activates a receptor complex consisting of a Frizzled (Fzd) receptor family member and low-density lipoprotein (LDL) receptor-related protein 5 or 6 (LRP5/6). To form the receptor complex that binds the Fzd ligands, Fzd receptors interact with LRP5/6, single pass transmembrane proteins with four extracellular EGF-like domains separated by six YWTD amino acid repeats (Johnson et al., 2004, J. Bone Mineral Res. 19:1749). The canonical Wnt signaling pathway activated upon receptor binding is mediated by the cytoplasmic protein Dishevelled (Dvl) interacting directly with the Fzd receptor and results in the cytoplasmic stabilization and accumulation of beta-catenin. In the absence of a Wnt signal, beta-catenin is localized to a cytoplasmic destruction complex that includes the tumor suppressor proteins adenomatous polyposis coli (APC) and Axin. These proteins function as critical scaffolds to allow glycogen synthase kinase (GSK)-3beta to bind and phosphorylate beta-catenin, marking it for degradation via the ubiquitin/proteasome pathway. Activation of Dvl results in the dissociation of the destruction complex. Accumulated cytoplasmic beta-catenin is then transported into the nucleus where it interacts with the DNA-binding proteins of the TCF/LEF family to activate transcription.

The non-canonical WNT pathway is regulated by three of these WNT ligands—WNT4, WNT5a, and WNT11. These ligands bind to the WNT receptor Frizzled in the absence of the co-receptors (LRP5/6). This leads to the activation of the RHO GTPase and ROCK kinase without activating cytoplasmic beta-catenin. ROCK regulates cytoskeleton to regulate apical-basal polarity of the cell. Because of competition for the same receptor, non-canonical WNT ligands also lead to inhibition of canonical WNT signaling.

Xeno-Free (XF): In relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the Xeno-free matrix may be essentially free of any non-human animal-derived components, therefore excluding mouse feeder cells or MATRIGEL™. MATRIGEL™ is a solubilized basement membrane preparation extracted from the Engelbreth-Holm-Swarm (EHS) mouse sarcoma, a tumor rich in extracellular matrix proteins to include laminin (a major component), collagen IV, heparan sulfate proteoglycans, and entactin/nidogen.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” “About” indicates within five percent, unless otherwise indicated. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Tissue Replacement Implants and Methods for Production

The cells in the retina that are directly sensitive to light are the photoreceptor cells. Photoreceptors are photosensitive neurons in the outer part of the retina and can be either rods or cones. In the process of phototransduction, the photoreceptor cells convert incident light energy focused by the lens to electric signals which are then sent via the optic nerve to the brain. Vertebrates have two types of photoreceptor cells including cones and rods. Cones are adapted to detect fine detail, central and color vision and function well in bright light. Rods are responsible for peripheral and dim light vision. Neural signals from the rods and cones undergo processing by other neurons of the retina.

The retinal pigment epithelium acts as a barrier between the bloodstream and the retina and closely interacts with photoreceptors in the maintenance of visual function. The retinal pigment epithelium is composed of a single layer of hexagonal cells that are densely packed with granules of melanin that absorbs light energy that arrives to the retina. The main functions of the specialized RPE cells include: transport of nutrients such as glucose, retinol, and fatty acids from the blood to the photoreceptors; transport of water, metabolic end products, and ions from the subretinal space to the blood; absorption of light and protection against photooxidation; reisomerization of all-trans-retinol into 11-cis-retinal; phagocytosis of shed photoreceptor membranes; and secretion of various essential factors for the structural integrity of the retina.

Dysfunction, injury, and loss of RPE cells are factors of many eye diseases and disorders including age-related macular degeneration (AMD) and hereditary macular degenerations such as Best disease, and retinitis pigmentosa. Other diseases are discussed below. Damage to the retina, such as from physical injury, also requires treatment. Disclosed herein is a tissue replacement implant comprising RPE cells that can be introduced locally into the eye of those in need of treatment. The tissue replacement implant improves retinal function and prevent blindness stemming from such conditions.

In some embodiments, disclosed are tissue replacement implants including RPE cells and methods for producing these tissue implants. These tissue replacement implants can be used for treating a retinal degenerative disease, retinal or retinal pigment epithelium dysfunction, retinal degradation, retinal damage, or loss of retinal pigment epithelium in a subject. These RPE cells can be produced, for example, using the methods disclosed in PCT Publication No. WO2014/121077 and PCT Publication No. WO 2017/044483, which are both incorporated by reference herein.

In some embodiments, the RPE cells are autologous to a subject that is being treated using the tissue implant. Autologous cells include cells from the same subject that are modified, such as to correct a mutation in a gene of interest, to express a heterologous protein, or to silence the expression of a mutant gene. In other embodiments, the RPE cells are allogeneic to the subject being treated using the tissue implant. In some non-limiting examples, the RPE cells are MHC matched to the subject being treated using the tissue implant. The RPE cells can be derived from a single subject or from multiple subjects, such as 2, 3, 4, or 5 subjects. In further embodiments, the RPE cells are produced from pluripotent stem cells. Disclosed are tissue implants and methods for producing tissue replacement implants including the RPE cells.

### A. Pluripotent Stem Cells

### 1. Embryonic Stem Cells

ES cells are derived from the inner cell mass of blastocysts and have a high in vitro differentiating capability. ES cells can be isolated by removing the outer trophectoderm layer of a developing embryo, then culturing the inner mass cells on a feeder layer of non-growing cells. The replated cells can continue to proliferate and produce new colonies of ES cells which can be removed, dissociated, replated again and allowed to grow. This process of “subculturing” undifferentiated ES cells can be repeated a number of times to produce cell lines containing undifferentiated ES cells (U.S. Pat. Nos. 5,843,780; 6,200,806; 7,029,913). ES cells have the potential to proliferate while maintaining their pluripotency. For example, ES cells are useful in research on cells and on genes which control cell differentiation. The pluripotency of ES cells combined with genetic manipulation and selection can be used for gene analysis studies in vivo via the generation of transgenic, chimeric, and knockout mice.

Methods for producing mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be produced or derived from a zygote or blastocyst-staged mammalian embryo produced by the fusion of a sperm and egg cell, nuclear transfer, pathogenesis, or the reprogramming of chromatin and subsequent incorporation of the reprogrammed chromatin into a plasma membrane to produce an embryonic cell by previously described methods (Thomson and Marshall, 1998; Reubinoff et al., 2000). In one method, human blastocysts are exposed to anti-human serum, and trophectoderm cells are lysed and removed from the inner cell mass which is cultured on a feeder layer of mouse embryonic fibroblasts. Further, clumps of cells derived from the inner cell mass are chemically or mechanically dissociated, replated, and colonies with undifferentiated morphology are selected by micropipette, dissociated, and replated (U.S. Pat. No. 6,833,269). In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as MATRIGEL® or laminin in the presence of “conditioned” medium containing basic fibroblast growth factor (Xu et al., 2001).

ES cells can also be derived from other organisms including rhesus monkey and marmoset by previously described methods (Thomson, and Marshall, 1998; Thomson et al., 1995; Thomson and Odorico, 2000), as well as from established mouse and human cell lines. For example, established human ES cell lines include MAOI, MA09, ACT-4, HI, H7, H9, H13, H14 and ACT30. As a further example, mouse ES cell lines that have been established include the CGR8 cell line established from the inner cell mass of the mouse strain 129 embryos, and cultures of CGR8 cells can be grown in the presence of LIF without feeder layers.

ES stem cells can be detected by protein markers including transcription factor Oct4, alkaline phosphatase (AP), stage-specific embryonic antigen SSEA-1, stage-specific embryonic antigen SSEA-3, stage-specific embryonic antigen SSEA-4, transcription factor NANOG, tumor rejection antigen 1-60 (TRA-1-60), tumor rejection antigen 1-81 (TRA-1-81), SOX2, or REX1.

a. Somatic Cell Nuclear Transfer

Pluripotent stem cells can be prepared through the method of somatic cell nuclear transfer. Somatic cell nuclear transfer involves the transfer of a donor nucleus into a spindle-free oocyte. In one method, donor fibroblast nuclei from skin fibroblasts of a primate are introduced into the cytoplasm of spindle-free, mature metaphase II primate ooctyes by electrofusion (Byrne et al., 2007). The fused oocytes are activated by exposure to ionomycin, and then incubated until the blastocyst stage. The inner cell mass of selected blastocysts are then cultured to produce embryonic stem cell lines. The embryonic stem cell lines show normal ES cell morphology, express various ES cell markers, and differentiate into multiple cell types both in vitro and in vivo.

### 2. Induced Pluripotent Stem Cells

The induction of pluripotency was originally achieved in 2006 using mouse cells (Yamanaka et al. 2006) and in 2007 using human cells (Yu et al. 2007; Takahashi et al. 2007) by reprogramming of somatic cells via the introduction of transcription factors that are linked to pluripotency. Pluripotent stem cells can be maintained in an undifferentiated state and are capable of differentiating into almost any cell type. The use of iPSCs circumvents most of the ethical and practical problems associated with large-scale clinical use of ES cells, and patients with iPSC-derived autologous transplants may not require lifelong immunosuppressive treatments to prevent graft rejection.

With the exception of germ cells, any cell can be used as a starting point for iPSCs. For example, cell types could be keratinocytes, fibroblasts, hematopoietic cells, mesenchymal cells, liver cells, or stomach cells. The cells can be a multipotent cells, such as but not limited to a hematopoietic stem cell, such as, but no limited to, CD34+ cells. T cells may also be used as a source of somatic cells for reprogramming (U.S. Pat. No. 8,741,648). There is no limitation on the degree of cell differentiation or the age of an animal from which cells are collected; even undifferentiated progenitor cells (including somatic stem cells) and finally differentiated mature cells can be used as sources of somatic cells in the methods disclosed herein. In one embodiment, the somatic cell is itself a RPE cells such as a human RPE cell. The RPE cell can be an adult or a fetal RPE cell. iPSCs can be grown under conditions that are known to differentiate human ES cells into specific cell types, and express human ES cell markers including: SSEA-1, SSEA-3, SSEA-4, TRA-1-60, and TRA-1-81.

Somatic cells and pluripotent stem cells, such as CD34+ cells, can be reprogrammed to produce induced pluripotent stem cells (iPSCs) using methods known to one of skill in the art. One of skill in the art can readily produce induced pluripotent stem cells, see for example, Published U.S. Patent Application No. 20090246875, Published U.S. Patent Application No. 2010/0210014; Published U.S. Patent Application No. 20120276636; U.S. Pat. Nos. 8,058,065; 8,129,187; 8,278,620; PCT Publication NO. WO 2007/069666 A1, and U.S. Pat. No. 8,268,620, which are incorporated herein by reference. Generally, nuclear reprogramming factors are used to produce pluripotent stem cells from a somatic cell. In some embodiments, at least three, or at least four, of Klf4, c-Myc, Oct3/4, Sox2, Nanog, and Lin28 are utilized. In other embodiments, Oct3/4, Sox2, c-Myc and Klf4 are utilized.

The cells are treated with a nuclear reprogramming substance, which is generally one or more factor(s) capable of inducing an iPSC from a somatic cell or a nucleic acid that encodes these substances (including forms integrated in a vector). The nuclear reprogramming substances generally include at least Oct3/4, Klf4 and Sox2 or nucleic acids that encode these molecules. A functional inhibitor of p53, L-myc or a nucleic acid that encodes L-myc, and Lin28 or Lin28b or a nucleic acid that encodes Lin28 or Lin28b, can be utilized as additional nuclear reprogramming substances. Nanog can also be utilized for nuclear reprogramming. As disclosed in published U.S. Patent Application No. 20120196360, exemplary reprogramming factors for the production of iPSCs include (1) Oct3/4, Klf4, Sox2, L-Myc (Sox2 can be replaced with Sox1, Sox3, Sox15, Sox17 or Sox18; Klf4 is replaceable with Klf1, Klf2 or Klf5); (2) Oct3/4, Klf4, Sox2, L-Myc, TERT, SV40 Large T antigen (SV40LT); (3) Oct3/4, Klf4, Sox2, L-Myc, TERT, human papilloma virus (HPV)16 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, Bmil; (7) Oct3/4, Klf4, Sox2, L-Myc, Lin28; (8) Oct3/4, Klf4, Sox2, L-Myc, Lin28, SV40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb is replaceable with Esrrg); (12) Oct3/4, Klf4, Sox2; (13) Oct3/4, Klf4, Sox2, TERT, SV40LT; (14) Oct3/4, Klf4, Sox2, TERT, HP VI 6 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, Bmil; (18) Oct3/4, Klf4, Sox2, Lin28 (19) Oct3/4, Klf4, Sox2, Lin28, SV40LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb is replaceable with Esrrg). In one non-limiting example, Oct3/4, Klf4, Sox2, and c-Myc are utilized. In other embodiments, Oct4, Nanog, and Sox2 are utilized, see for example, U.S. Pat. No. 7,682,828, which is incorporated herein by reference. These factors include, but are not limited to, Oct3/4, Klf4 and Sox2. In other examples, the factors include, but are not limited to Oct 3/4, Klf4 and Myc. In some non-limiting examples, Oct3/4, Klf4, c-Myc, and Sox2 are utilized. In other non-limiting examples, Oct3/4, Klf4, Sox2 and Sal 4 are utilized. Factors like Nanog, Lin28, Klf4, or c-Myc can increase reprogramming efficiency and can be expressed from several different expression vectors. For example, an integrating vector such as the EBV element-based system can be used (U.S. Pat. No. 8,546,140). In a further aspect, reprogramming proteins could be introduced directly into somatic cells by protein transduction. Reprogramming may further comprise contacting the cells with one or more signaling receptors including glycogen synthase kinase 3 (GSK-3) inhibitor, a mitogen-activated protein kinase (MEK) inhibitor, a transforming growth factor beta (TGF-β) receptor inhibitor or signaling inhibitor, leukemia inhibitory factor (LIF), a p53 inhibitor, an NF-kappa B inhibitor, or a combination thereof. Those regulators may include small molecules, inhibitory nucleotides, expression cassettes, or protein factors. It is anticipated that virtually any iPS cells or cell lines may be used.

Mouse and human cDNA sequences of these nuclear reprogramming substances are available with reference to the NCBI accession numbers mentioned in WO 2007/069666, which is incorporated herein by reference. Methods for introducing one or more reprogramming substances, or nucleic acids encoding these reprogramming substances, are known in the art, and disclosed for example, in published U.S. Patent Application No. 2012/0196360 and U.S. Pat. No. 8,071,369, which both are incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used.

In certain embodiments, undefined conditions may be used; for example, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR™ medium (Ludwig et al., 2006a; Ludwig et al., 2006b) or E8™ medium (Chen et al., 2011).

In some embodiments, the iPSCs can be modified, such as to express an exogenous gene, increase expression of an endogenous gene, increase copy number of a gene, to correct a gene mutation, or to silence the expression of a mutant gene. In some specific non-limiting examples, a mutation or a deletion in an endogenous gene is corrected. The gene can encode, for example, retinoid isomerohydrase (RPE65), bestrophin (BEST)1, MER tyrosine kinase proto-oncogene (MERTK), RAB escort protein (REP1), cellular retinaldyehyde binding protein (CRALBP), pre-mRNA processing factor (PPRF), complement factor H (CFH), complement component 3a receptor (C3aR)1, complement component 5 receptor (C5aR1), vascular endothelial growth factor (VEGF), pigment epithelium-derived factor (PEDF), complement factor I (CFI), complement factor 2B (C2B), ATP-binding cassette, subfamily A, member 4 (ABCA4), ATP-binding cassette, subfamily A, member 1 (ABCA1), membrane-type frizzled related protein (MFRP), C1q and tumor necrosis factor related protein 5 (ClqTNF5), spermatogenesis-associated protein 7 (SPATA7), centrosomal protein, 290 kd (CEP290), myosin VIIA (MYO7A), cilliary neurotrophic factor (CNTF), FMS-related tyrosine kinase 1 (FLT-1), Usher syndrome, type I (USH1A), tyrosinase, plasminogen (PLG), collagen, type XVIII, alpha-1 (COL18A1), TTRA serine peptidase (HTRA)1, ARMS2, tissue inhibitor of metalloproteinase (TIMP)3, epithelial growth factor (EGF) containing fibulin-like extracellular matrix protein (EFEMP)1, microphthalmia-associated transcription factor (MITF), transcription factor EC (TFEC), orthodenticle, drosophila, homolog of, 2 (OTX2), zinc finger protein 503 (ZNF503 or NLZ2). In one non-limiting example, the gene is RPE65. In another non-limiting example, the gene is BEST1. If a further non-limiting example, the gene encodes METRK. Methods for performing gene editing in iPSCs are disclosed, for example, in Hockenmeyer and Jaenisch, “Induced Pluripotent Stem Cell Meets Genome Editing,” Cell Stem Cell 18: 573-586, 2016, incorporated herein by reference. Any of the methods disclosed therein are of use. The method can include the use of a viral vector, such as an adeno-associated viral vector or a lentiviral vector ending a transgene of interest. The method can include the use of CRISPR/Cas9, TALEN nuclease, Zinc-finger nuclease, lentiviral mediated correction, adeno-associated virus mediated correction, shRNA, siRNA, or F-prime editing.

In some embodiments, the iPSC can be modified to express exogenous nucleic acids, such as to include a tyrosinase enhancer operably linked to a promoter and a nucleic acid sequence encoding a first marker. The tyrosinase gene is disclosed, for example, in GENBANK® Accession No. 22173, as available on Jan. 1, 2013. This sequence aligns to chromosome 7 of mouse strain C57BL/6 location 5286971-5291691 (invert orientation). A 4721 base pair sequence is sufficient for expression in RPE cells, see Murisier et al., Dev. Biol. 303: 838-847, 2007, which is incorporated herein by reference. This construct is expressed in retinal pigment epithelial cells. Other enhancers can be utilized. Other RPE-specific enhancers include D-MITF, DCT, TYRP1, RPE65, VMD2, MERTK, MYRIP, and RAB27A. Suitable promoters include, but are not limited to, any promoter expressed in retinal pigment epithelial cells including the tyrosinase promoter. The construct can also include other elements, such as a ribosome binding site for translational initiation (internal ribosomal binding sequences), and a transcription/translation terminator. Generally, it is advantageous to transfect cells with the construct. Suitable vectors for stable transfection include, but are not limited to retroviral vectors, lentiviral vectors and Sendai virus.

Plasmids have been designed with a number of goals in mind, such as achieving regulated high copy number and avoiding potential causes of plasmid instability in bacteria, and providing means for plasmid selection that are compatible with use in mammalian cells, including human cells. There are dual requirements of plasmids for use in human cells. First, they are suitable for maintenance and fermentation in E. coli, so that large amounts of DNA can be produced and purified. Second, they are safe and suitable for use in human patients and animals. The first requirement calls for high copy number plasmids that can be selected for and stably maintained relatively easily during bacterial fermentation. The second requirement calls for attention to elements such as selectable markers and other coding sequences. In some embodiments plasmids that encode a marker are composed of: (1) a high copy number replication origin, (2) a selectable marker, such as, but not limited to, the neo gene for antibiotic selection with kanamycin, (3) transcription termination sequences, including the tyrosinase enhancer and (4) a multicloning site for incorporation of various nucleic acid cassettes; and (5) a nucleic acid sequence encoding a marker operably linked to the tyrosinase promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, which are incorporated herein by reference.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector.

In some embodiments, the cells are transfected with a nucleic acid molecule encoding a marker. Markers include, but are not limited to, fluorescence proteins (for example, green fluorescent protein or red fluorescent protein), enzymes (for example, horse radish peroxidase or alkaline phosphatase or firefly/renilla luciferase or nanoluc), or other proteins. A marker may be a protein (including secreted, cell surface, or internal proteins; either synthesized or taken up by the cell); a nucleic acid (such as an mRNA, or enzymatically active nucleic acid molecule) or a polysaccharide. Included are determinants of any such cell components that are detectable by antibody, lectin, probe or nucleic acid amplification reaction that are specific for the marker of the cell type of interest. The markers can also be identified by a biochemical or enzyme assay or biological response that depends on the function of the gene product. Nucleic acid sequences encoding these markers can be operably linked to the tyrosinase enhancer. In addition, other genes can be included, such as genes that may influence stem cell to RPE differentiation, or RPE function, or physiology, or pathology. Thus, in some embodiments, a nucleic acid is included that encodes one or more of MITF, PAX6, TFEC, OTX2, LHX2, VMD2, CFTR, RPE65, MFRP, CTRP5, CFH, C3, C2B, APOE, APOB, mTOR, FOXO, AMPK, SIRT1-6, HTRP1, ABCA4, TIMP3, VEGFA, CFI, TLR3, TLR4, APP, CD46, BACE1, ELOLV4, ADAM 10, CD55, CD59, and ARMS2.

a. MHC Haplotype Matching

Major Histocompatibility Complex is the main cause of immune-rejection of allogeneic organ transplants. There are three major class I MHC haplotypes (A, B, and C) and three major MHC class II haplotypes (DR, DP, and DQ). The HLA loci are highly polymorphic and are distributed over 4 Mb on chromosome 6. The ability to haplotype the HLA genes within the region is clinically important since this region is associated with autoimmune and infectious diseases and the compatibility of HLA haplotypes between donor and recipient can influence the clinical outcomes of transplantation. HLAs corresponding to MHC class I present peptides from inside the cell and HLAs corresponding to MHC class II present antigens from outside of the cell to T-lymphocytes. Incompatibility of MHC haplotypes between the graft and the host triggers an immune response against the graft and leads to its rejection. Thus, a subject can be treated with an immunosuppressant to prevent rejection. HLA-matched stem cell lines may overcome the risk of immune rejection.

Because of the importance of HLA in transplantation, the HLA loci are usually typed by serology and PCR for identifying favorable donor-recipient pairs. Serological detection of HLA class I and II antigens can be accomplished using a complement mediated lymphocytotoxicity test with purified T or B lymphocytes. This procedure is predominantly used for matching HLA-A and -B loci. Molecular-based tissue typing can often be more accurate than serologic testing. Low resolution molecular methods such as SSOP (sequence specific oligonucleotide probes) methods, in which PCR products are tested against a series of oligonucleotide probes, can be used to identify HLA antigens, and currently these methods are the most common methods used for Class II-HLA typing. High resolution techniques such as SSP (sequence specific primer) methods which utilize allele specific primers for PCR amplification can identify specific MHC alleles.

MHC compatibility between a donor and a recipient increases significantly if the donor cells are HLA homozygous, i.e. contain identical alleles for each antigen-presenting protein. Most individuals are heterozygous for MHC class I and II genes, but certain individuals are homozygous for these genes. These homozygous individuals can serve as super donors and grafts generated from their cells can be transplanted in all individuals that are either homozygous or heterozygous for that haplotype. Furthermore, if homozygous donor cells have a haplotype found in high frequency in a population, these cells may have application in transplantation therapies for a large number of individuals.

Accordingly, iPSCs can be produced from cells, such as CD34+ cells, of the subject to be treated, or another subject with the same or substantially the same HLA type as that of the patient. In one case, the major HLAs (e.g., the three major loci of HLA-A, HLA-B and HLA-DR) of the donor are identical to the major HLAs of the recipient. In some cases, the somatic cell donor may be a super donor; thus, iPSCs derived from a MHC homozygous super donor may be used to generate RPE cells. Thus, the iPSCs derived from a super donor may be transplanted in subjects that are either homozygous or heterozygous for that haplotype. For example, the iPSCs can be homozygous at two HLA alleles such as HLA-A and HLA-B. As such, iPSCs produced from super donors can be used in the methods disclosed herein, to produce RPE cells that can potentially “match” a large number of potential recipients.

b. Episomal Vectors

In certain aspects, reprogramming factors are expressed from expression cassettes comprised in one or more exogenous episomal genetic elements (see U.S. Patent Publication 2010/0003757, incorporated herein by reference). Thus, iPSCs can be essentially free of exogenous genetic elements, such as from retroviral or lentiviral vector elements. These iPSCs are prepared by the use of extra-chromosomally replicating vectors (i.e., episomal vectors), which are vectors capable of replicating episomally to make iPSCs essentially free of exogenous vector or viral elements (see U.S. Pat. No. 8,546,140, incorporated herein by reference; Yu et al., 2009). A number of DNA viruses, such as adenoviruses, simian virus 40 (SV40) or bovine papilloma virus (BPV), or budding yeast ARS (Autonomously Replicating Sequences)-containing plasmids replicate extra-chromosomally or episomally in mammalian cells. These episomal plasmids are intrinsically free from all these disadvantages (Bode et al., 2001) associated with integrating vectors. For example, a lymphotrophic herpes virus-based including or Epstein Barr Virus (EBV) as defined above may replicate extra-chromosomally and help deliver reprogramming genes to somatic cells. Useful EBV elements are OriP and EBNA-1, or their variants or functional equivalents. An additional advantage of episomal vectors is that the exogenous elements will be lost with time after being introduced into cells, leading to self-sustained iPSCs essentially free of these elements.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV); herpes virus saimiri (HS) and Marek's disease virus (MDV). Additional sources of episome-based vectors are contemplated, such as yeast ARS, adenovirus, SV40, or BPV.

### B Retinal Pigment Epithelial Cells

RPE ells are utilized in the disclosed tissue implants. In some embodiments, cells can be produced from stem cells, such as ESCs or iPSCs.

The retinal pigment epithelium expresses markers such as cellular retinaldehyde-binding protein (CRALBP), RPE65, best vitelliform macular dystrophy gene (VMD2), and pigment epithelium derived factor (PEDF). Malfunction of the retinal pigment epithelium is associated with a number of vision-altering conditions, such as retinal pigment epithelium detachment, dysplasia, atrophy, retinopathy, retinitis pigmentosa, macular dystrophy, or degeneration.

Retinal pigment epithelial (RPE) cells can be characterized based upon their pigmentation, epithelial morphology, and apical-basal polarity. Differentiated RPE cells can be visually recognized by their cobblestone morphology and the initial appearance of pigment. In addition, differentiated RPE cells have transepithelial resistance/TER, and trans-epithelial potential/TEP across the monolayer (TER>200 Oms*cm²; TEP>2 mV), transport fluid and CO₂ from the apical to basal side, and regulate a polarized secretion of cytokines.

RPE cells express several proteins that can serve as markers for detection by the use of methodologies, such as immunocytochemistry, Western blot analysis, flow cytometry, and enzyme-linked immunoassay (ELISA). For example, RPE-specific markers may include: cellular retinaldehyde binding protein (CRALBP), microphthalmia-associated transcription factor (MITF), tyrosinase-related protein 1 (TYRP-1), retinal pigment epithelium-specific 65 kDa protein (RPE65), premelanosome protein (PMEL17), bestrophin 1 (BEST1), and c-mer proto-oncogene tyrosine kinase (MERTK). RPE cells do not express (at any detectable level) the embryonic stem cells markers Oct-4, nanog or Rex-2. Specifically, expression of these genes is approximately 100-1000 fold lower in RPE cells than in ES cells or iPSCs, when assessed by quantitative RT-PCR.

RPE cell markers may be detected at the mRNA level, for example, by reverse transcriptase polymerase chain reaction (RT-PCR), Northern blot analysis, or dot-blot hybridization analysis using sequence-specific primers in standard amplification methods using publicly available sequence data (GENBANK®). Expression of tissue-specific markers as detected at the protein or mRNA level is considered positive if the level is at least or about 2-, 3-, 4-, 5-, 6-, 7-, 8-, or 9-fold, and more particularly more than 10-, 20-, 30, 40-, 50-fold or higher above that of a control cell, such as an undifferentiated pluripotent stem cell or other unrelated cell type.

Exemplary methods for producing RPE from iPSCs and ESCs are disclosed below. A description of inhibitors that can be used in the media are disclosed at the end of this section. It should be noted that any of the specific inhibitors can be used wherein a reference is made to the class of inhibitors.

1. Derivation of RPE Cells from Embryoid Bodies of PSCs

iPSCs, such as, but no limited to, iPSC produced form CD34+ cells, can be reprogrammed using well-known reprogramming factors to produce RPE cells. PCT Publication No. 2014/121077, incorporated by reference herein in its entirety, discloses methods wherein embryoid bodies (EBs) produced from iPSCs are treated with Wnt and Nodal antagonists in suspension culture to induce expression of markers of retinal progenitor cells. This publication discloses methods wherein RPE cells are derived from iPSCs through a process of differentiation of EBs of the iPSCs into cultures highly enriched for RPE cells. For example, embryoid bodies are produced from iPSCs by the addition of a rho-associated coiled-coil kinase (ROCK) inhibitor and cultured in a first medium comprising two WNT pathway inhibitors and a Nodal pathway inhibitor. Further, the EBs are plated on a MATRIGEL® coated tissue culture in a second medium that does not comprise basic fibroblast growth factor (bFGF), comprises a Nodal pathway inhibitor, comprises about 20 ng to about 90 ng of Noggin, and comprises about 1 to about 5% knock out serum replacement to form differentiating RPE cells. The differentiating RPE cells are cultured in a third medium comprising ACTIVIN and/or WNT3a. The RPE cells are then cultured in RPE medium that includes about 5% fetal serum, a canonical WNT inhibitor, a non-canonical WNT inhibitor, and inhibitors of the Sonic Hedgehog and FGF pathways to produce human RPE cells.

There are several disadvantages in the use of EBs for the production of differentiated cell type. For example, the production of EBs is a non-consistent and non-reproducible process as the efficiency varies. The size and shape of EBs produced from iPSCs or ES cells is not homogenous, and the production of EBS also involves a rate-limiting centrifugation treatment. The present disclosure provides methods that allow large-scale production of iPSC- or ES-derived cells needed for clinical, research or therapeutic applications that are independent of EBs.

2. Derivation of RPE Cells from Essentially Single Cell PSCs

In some embodiments, RPE cells are produced from an essentially single cell suspension of pluripotent stem cells (PSCs) such as human iPSCs. In some embodiments, the PSCs are cultured to pre-confluency to prevent any cell aggregates. In certain aspects, the PSCs are dissociated by incubation with a cell dissociation enzyme, such as exemplified by TRYPSIN™ or TRYPLE™. PSCs can also be dissociated into an essentially single cell suspension by pipetting. In addition, Blebbistatin (e.g., about 2.5 μM) can be added to the medium to increase PSC survival after dissociation into single cells while the cells are not adhered to a culture vessel. A ROCK inhibitor instead of Blebbistatin may alternatively used to increase PSC survival after dissociated into single cells.

In order to efficiently differentiate RPE cells from the single cell PSCs, an accurate count of the input density can increase RPE differentiation efficiency. Thus, the single cell suspension of PSCs is generally counted before seeding. For example, the single cell suspension of PSCs is counted by a hemocytometer or an automated cell counter, such as VICELL® or TC20. The cells may be diluted to a cell density of about 10,000 to about 500,000 cells/mL, about 50,000 to about 200,000 cells/mL, or about 75,000 to about 150,000 cells/mL. In a non-limiting example, the single cell suspension of PSCs is diluted to a density of about 100,000 cells/mL in a fully defined cultured medium such as ESSENTIAL 8™ (E8™) medium.

Once a single cell suspension of PSCs is obtained at a known cell density, the cells are generally seeded in an appropriate culture vessel, such as a tissue culture plate, such as a flask, 6-well, 24-well, or 96-well plate. A culture vessel used for culturing the cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CELLSTACK® chambers, culture bag, and roller bottle, as long as it is capable of culturing the stem cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system ex vivo that supports a biologically active environment such that cells can be propagated. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable

In certain aspects, the PSCs, such as iPSCs, are plated at a cell density appropriate for efficient differentiation. Generally, the cells are plated at a cell density of about 1,000 to about 75,000 cells/cm2, such as of about 5,000 to about 40,000 cells/cm2. In a 6 well plate, the cells may be seeded at a cell density of about 50,000 to about 400,000 cells per well. In exemplary methods, the cells are seeded at a cell density of about 100,000, about 150,00, about 200,000, about 250,000, about 300,000 or about 350,000 cells per well, such as about 200,00 cells per well.

The PSCs, such as iPSCs, are generally cultured on culture plates coated by one or more cellular adhesion proteins to promote cellular adhesion while maintaining cell viability. For example, preferred cellular adhesion proteins include extracellular matrix proteins such as vitronectin, laminin, collagen and/or fibronectin which may be used to coat a culturing surface as a means of providing a solid support for pluripotent cell growth. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. In exemplary methods, the PSCs are grown on culture plates coated with vitronectin or fibronectin. In some embodiments, the cellular adhesion proteins are human proteins.

The extracellular matrix (ECM) proteins may be of natural origin and purified from human or animal tissues or, alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition includes synthetically generated peptide fragments of fibronectin or recombinant fibronectin. In some embodiments, the matrix composition is xeno-free. For example, in the xeno-free matrix to culture human cells, matrix components of human origin may be used, wherein any non-human animal components may be excluded.

In some aspects, the total protein concentration in the matrix composition may be about 1 ng/mL to about 1 mg/mL. In some preferred embodiments, the total protein concentration in the matrix composition is about 1 g/mL to about 300 g/mL. In more preferred embodiments, the total protein concentration in the matrix composition is about 5 g/mL to about 200 g/mL.

a. Culture Conditions

Cells, such as RPE cells or PSC, can be cultured with the nutrients necessary to support the growth of each specific population of cells. Generally, the cells are cultured in growth media including a carbon source, a nitrogen source and a buffer to maintain pH. The medium can also contain fatty acids or lipids, amino acids (such as non-essential amino acids), vitamin(s), growth factors, cytokines, antioxidant substances, pyruvic acid, buffering agents, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. In some embodiments, the PSCs are cultured in a fully defined and feeder free media.

Accordingly, the single cell PSCs are generally cultured in a fully defined culture medium after plating. In certain aspects, about 18-24 hours after seeding, the medium is aspirated and fresh medium, such as E8™ medium, is added to the culture. In certain aspects, the single cell PSCs are cultured in the fully defined culture medium for about 1, 2 or 3 days after plating. In some non-limiting examples, the single cells PSCs are cultured in the fully defined culture medium for about 2 days before proceeding with the differentiation process.

In some embodiments, the medium may contain alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco). The medium can be serum free.

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO2 concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

b. Differentiation Media
i. Retinal Induction Medium

After the single cell PSCs have adhered to the culture plate, the cells are preferably cultured in Retinal Induction Medium to start the differentiation process into retinal lineage cells. The Retinal Induction Medium (RIM) comprises a WNT pathway inhibitor and can result in the differentiation of PSCs to retinal lineage cells. The RIM additionally comprises a TGFβ pathway inhibitor and a BMP pathway inhibitor.

The RIM can include DMEM and F12 at about a 1:1 ratio. In exemplary methods, a WNT pathway inhibitor is included in the RIM, such as CKI-7, a BMP pathway inhibitor is included, such as LDN193189, and the TGFβ pathway inhibitor is included, such as SB431542. For example, the RIM comprises about 5 nM to about 50 nM, such as about 10 nM, of LDN193189, about 0.1 μM to about 5 μM, such as about 0.5 μM, of CKI-7, and about 0.5 μM to about 10 μM, such as about 1 μM, of SB431542. Additionally, the RIM can include knockout serum replacement, such as about 1% to about 5%, MEM non-essential amino acids (NEAA), sodium pyruvate, N-2 supplement, B-27 supplement, ascorbic acid, and insulin growth factor 1 (IGF1). In some embodiments, the IGF1 is animal free IGF1 (AF-IGF1) and is comprised in the RIM from about 0.1 ng/mL to about 10 ng/mL, such as about 1 ng/mL. The media is such as aspirated each day and replaced with fresh RIM. The cells are generally cultured in the RIM for about 1 to about 5 days, such as about 1, 2, 3, 4 or 5 days, such as for about 2 days to produce retinal lineage cells.

ii. Retinal Differentiation Medium

The retinal lineage cells can then be cultured in Retinal Differentiation Medium (RDM) for further differentiation. The RDM comprises a WNT pathway inhibitor, a BMP pathway inhibitor, a TGFβ pathway inhibitor and a MEK inhibitor. In one embodiment, the RDM comprises a WNT pathway inhibitor, such as CKI-7, a BMP pathway inhibitor, such as LDN193189, a TGFβ pathway inhibitor, such as SB431542, and a MEK inhibitor, such as PD0325901. Alternatively, the RDM can comprise a WNT pathway inhibitor, a BMP pathway inhibitor, a TGFβ pathway inhibitor and a bFGF inhibitor. Generally, the concentrations of the Wnt pathway inhibitor, BMP pathway inhibitor and TGFβ pathway inhibitor are higher in the RDM as compared to the RIM, such as about 9 to about 11 times higher, such as about 10 times higher. In exemplary methods, the RDM comprises about 50 nM to about 200 nM, such as about 100 nM of LDN193189, about 1 μM to about 10 μM, such as about 5 μM, of CKI-7, about 1 μM to about 50 μM, such as about 10 μM, of SB431542, and about 0.1 μM to about 10 μM, such as about 1 μM, 2 μM, 3 μM, 4 μM, 5 μM, 6 μM, 7 μM, 8 μM, or 9 μM of PD0325901.

Generally, the RDM comprises DMEM and F12 at about a 1:1 ratio, knockout serum replacement (e.g., about 1% to about 5%, such as about 1.5%), MEM NEAA, sodium pyruvate, N-2 supplement, B-27 supplement, ascorbic acid and IGF1 (e.g., about 1 ng/mL to about 50 ng/mL, such as about 10 ng/mL). In particular methods, the cells are given fresh RDM each day after aspiration of the media from the previous day. Generally, the cells are cultured in the RDM for about 2, 3, 4, 5, 6, 7, 8, 9, 10, 11, 12, 13, 14, 15, or 16 days, such as for about 7 days to derive differentiated retinal cells. iii. Retinal Medium

Next, the differentiated retinal cells can be even further differentiated by culturing the cells in Retinal Medium (RM). The Retinal Medium comprises Activin A and can additionally comprise Nicotinamide. The RM can comprise about 50 to about 200 ng/mL, such as about 100 ng/mL, of ACTIVIN A, and about 1 mM to about 50 mM, such as about 10 mM, of nicotinamide. Alternatively, the RM can comprise other TGF-β pathway activators such as GDF1 and/or WNT pathway activators such as WAY-316606, IQ1, QS11, SB-216763, BIO (6-bromoindirubin-3′-oxime), or 2-amino-4-[3,4-(methylenedioxy)benzyl-amino]-6-(3-methoxyphenyl) pyrimidine. Alternatively, the RM can additionally comprise WNT3a.

The RM can include DMEM and F12 at about a 1:1 ratio, knockout serum replacement at about 1% to about 5%, such as about 1.5%, MEM non-essential amino acids (NEAA), sodium pyruvate, N-2 supplement, B-27 supplement, and ascorbic acid. The medium can be changed daily with room temperature RM. The cells are generally cultured in the RM for about 8, 9, 10, 11, 12, 13, 14, 15, 16 or 17 days, such as for about 10 days to derive differentiating RPE cells. The retinal pigment epithelial cells produced by the methods disclosed herein can be cryopreserved, see for example, PCT Publication No. 2012/149484 A2, which is incorporated by reference herein. The cells can be cryopreserved with or without a substrate. In several embodiments, the storage temperature ranges from about −50° C. to about −60° C., about −60° C. to about −70° C., about −70° C. to about −80° C., about −80° C. to about −90° C., about −90° C. to about −100° C., and overlapping ranges thereof. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored 48 hours to about one week. In yet other embodiments, the cells are stored for about 1, 2, 3, 4, 5, 6, 7, or 8 weeks. In further embodiments, the cells are stored for 1, 2, 3, 4, 5, 67, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80, serum albumin, such as human or bovine serum albumin. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8%- to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C. minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C. The cells are then matured on a scaffold, as discussed below.

iv. RPE-Maturation Medium

For further differentiation of the RPE cells, the cells are preferably cultured in RPE Maturation Medium (RPE-MM). The RPE-Maturation Medium can comprise about 100 μg/mL to about 300 μg/mL, such as about 250 μg/mL, of taurine, about 10 μg/L to about 30 μg/L, such as about 20 μg/L, of hydrocortisone and about 0.001 μg/L to about 0.1 μg/L, such as about 0.013 μg/L, of triiodothyronine. Additionally, the RPE-MM can comprise MEM Alpha, N-2 supplement, MEM non-essential amino acids (NEAA), and sodium pyruvate, and fetal bovine serum (e.g., about 0.5% to about 10%, such as about 1% to about 5%). The medium can be changed every other day with room temperature RPE-MM. The cells are generally cultured in RPE-MM for about 5 to about 10 days, such as about 5 days. The cells can then be dissociated, such as with a cell dissociation enzyme, reseeded, and cultured for an additional period of time, such as an additional about 5 to about 30 days, such as about 15 to 20 days, for further differentiation into RPE cells. In further embodiments, the RPE-MM does not include a WNT pathway inhibitor. RPE cells can be cryopreserved at this stage.

b. Maturation of RPE Cells on the Scaffold and the Tissue Replacement Implant

The RPE cells can then be cultured in the RPE-MM for a continued period of time for maturation. In some embodiments, the RPE cells are grown in wells, such as a 6-well, 12-well, 24-well, or 10 cm plate. RPE cells can be maintained in RPE medium on a scaffold for about four to about ten weeks, such as for about six to eight weeks, such as for four, five, six, seven, or eight weeks. In some non-limiting examples, the RPE cells are cultured in a medium on a scaffold for about two to six weeks, such as about five weeks, to obtain mature and functional RPE cell monolayers. This culturing produces polarized RPE cells on the scaffold, which together form the tissue implant.

A variety of biological or synthetic solid matrix materials (i.e., solid support matrices, biological adhesives or dressings, and biological/medical scaffolds) are suitable for use as the scaffold. The matrix material is generally physiologically acceptable and suitable for use in vivo applications. Non-limiting examples of such physiologically acceptable materials include, but are not limited to, solid matrix materials that are biodegradable, such crosslinked or non-crosslinked alginate, hydrocolloid, foams, collagen gel, collagen sponge, polyglycolic acid (PGA) mesh. polyglactin (PGL) mesh, and bioadhesives (e.g., fibrin glue and fibrin gel). The polymer can be poly(DL)-lactic-co-glycolic) acid (PLGA) (see Lu et al., J. Biomater Sci Polym Ed. 9(11): 1187-205, 1998). In other embodiments, the matric includes poly(L-lactic acid) (PLLA) and poly(D,L-lactic-co-glycolic acid) (PLGA), such as with a co-polymer ratio of about 90:10, 75:25, 50:50, 25:75, 10:90 (PLLA:PLGA) (see Thomson et al., J. Biomed. Mater Res. A 95: 1233-42, 2010).

Suitable polymeric carriers also include porous meshes or sponges formed of synthetic or natural polymers. A non-limiting example is a polymeric hydrogel. Natural polymers that can be used include proteins such as collagen, albumin, and fibrin; and polysaccharides such as alginate and polymers of hyaluronic acid. Synthetic polymers can be biodegradable. Examples of biodegradable polymers include polymers of hydroxy acids such as polylactic acid (PLA), polyglycolic acid (PGA), and polylactic acid-glycolic acid (PLGA), polyorthoesters, polyanhydrides, polyphosphazenes, and combinations thereof.

In some embodiments, the scaffold is a PLGA scaffold, as disclosed below. PLGA is a copolymer of poly-lactic acid (PLA) and poly-glycolic acid (PGA). Poly-lactic acid contains an asymmetric α-carbon which is typically described as the D or L form in classical stereochemical terms and sometimes as R and S form, respectively. The enantiomeric forms of the polymer PLA are poly D-lactic acid (PDLA) and poly L-lactic acid (PLLA). PLGA is poly D, L-lactic-co-glycolic acid where D- and L-lactic acid forms are generally in equal ratio. PLGA biodegrades by hydrolysis of its ester linkages.

In some embodiments, the PLGA scaffold is cultured for a sufficient time such that the bulk of lactic acid release from the scaffold occurs in vitro. In some embodiments, greater than 50%, 60%, 70%, 80%, 90% or 95% of the lactic acid release occurs in vitro. The lactic acid release occurs over time. Thus, in some embodiments, maintaining the RPE in RPE medium on the scaffold for about four to about ten weeks, such as for about six to eight weeks, such as for four, five, six, seven, or eight weeks achieves this effect. In some non-limiting examples, culturing the RPE cells in a medium on a scaffold for about two to six weeks, such as about five weeks achieves this effect.

In some embodiments, the PLGA scaffold can have a DL-lactide/glycotide ratio of about 5:1 to about 1:5, such as about 4:1 to about 1:4, about 3:1o to about 3:3, about 2:1 to 1:2. In one specific non-limiting examples, the DL-lactide/glycotide ratio is 1:1. In this context, “about” indicates within 5%.

In more embodiments, the PLGA scaffold is about 10 to about 50 microns in thickness, such as about 20 to about 40 microns in thickness, such as about 20 to about 30 microns in thickness. The PLGA scaffold can be about 20, 21, 22, 23, 24, 25, 26, 27, 28, 29 or 30 microns in thickness. In this context, about indicates within 5%.

The scaffold has nanofibers that intersect each other, such that they intersect and form junctions. The scaffold can be treated to fuse fibers of the scaffold at the junctions of fiber intersections within the PLGA scaffold to increase mechanical strength. The average pore size is the space between the fibers in the PLGA scaffold.

In additional embodiments, the PLGA scaffold has a pore size of less than about 2 microns, such as less than about 1.5 microns, less than about 1.25 microns, or less than about 1 micron. In some embodiments, the PLGA scaffold has a pore size of about 0.5 microns to about 2 microns, about 0.5 to 1 microns, about 1 to about 2 microns. The PLGA scaffold can have a pore size of about 0.5, 0.75, 1, 1.25, 1.5, 1.75 or 2 microns. In this context, about indicates within 5%.

In further embodiments, the PLGA scaffold has a fiber diameter of about 100 to about 700 nm, such as about 150 to about 650 nm, such as about 200 to about 600 nm, such as about 300 to about 500 nm. In some embodiments, the PLGA scaffold has a fiber diameter of about 100, 150, 200, 250, 300, 350, 400, 450, 500, 550, 600 or 650 nm. In this context, “about” indicates within 5%.

It should be noted that any of the features of the PLGA scaffold of thickness, pore size and fiber diameter can be combined. One of skill in the art can understands that varying a DL-lactide/glycotide ratio will adjust pore and fiber diameter. Thus, one of skill in the art can produce PLGA scaffolds of the disclosed thickness, pore sizes and fiber diameters. Any features can be combined to arrive at specific combinations produced by varying DL-lactide/glycotide ratio. These are all understood to be disclosed herein. In a specific non-limiting example, the PLGA scaffold has a DL-lactide/glycotide ratio of 1:1, an average pore size of less than 1 microns, and a fiber diameter of 150 to 650 nm.

In some embodiments, the scaffold is treated with heat to fuse fibers of the scaffold at the junctions (fiber intersections) within the PLGA scaffold to increase mechanical strength of the PLGA scaffold. This heat treatment also reduces pore size by fusing the fibers at the junctions and thus allows the cells to form a monolayer on top of the scaffold. In some non-limiting examples, some embodiments, scaffold are place on an appropriate surface, for example, a metal surface such aluminum foil, such as in the form of an envelope, that is placed inside an oven set to the desired temperature for treatment. Suitable temperatures include about 35° C. to about 55° C., such as about 40° C. to about 50° C., such as about 43° C., 44° C., 45° C., 46° C., or 47° C. The scaffold can be heated for about 5 to about 20 minutes, such as about 10 to about 15 minutes, such as about 10, 11, 12, 13, 14 or 15 minutes. In one embodiment, the scaffold is treated at about 45° C. to for about 10 minutes. The temperature can then be increased relative to the first temperature, such as to about 50° C. to about 70° C., such as about 55° C. to about 60° C., such as about 55° C., 56° C., 57° C., 58° C., 59° C. or 60° C. The higher temperature treatment can be for about 45 minutes to about 75 minutes, such as about 50 minutes to about 70 minutes, or about 55 minutes to about 65 minutes. The higher temperature treatment can be applied for about 55, 56, 57, 58, 59, 60, 61, 62, 63, 64 or 65 minutes. In some embodiments, the scaffold is treated at about 60° C. for about 60 minutes. In one non-limiting example, the scaffold can be treated at about 45° C. to for about 10 minutes and then at about 60° C. for about 60 minutes. Following heat treatment, the scaffold can be stored.

In some embodiments, the PLGA scaffold is coated with vitronectin. In other embodiments, the PLGA scaffold is coated with an extracellular matrix or gelatin. This can occur after heating the scaffold, as disclosed above.

In other embodiments, the PLGA scaffold is coated with an extracellular matrix. An extracellular matrix is a complex mixture of structural and functional biomolecules and/or biomacromolecules including, but not limited to, structural proteins, specialized proteins, proteoglycans, glycosaminoglycans, and growth factors that surround and support cells within mammalian tissues and, unless otherwise indicated, is acellular. Extracellular matrices are disclosed, for example and without limitation, in U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666; each of which is incorporated by reference in its entirety). However, an ECM can be produced from any tissue, or from any in vitro source wherein the ECM is produced by cultured cells and comprises one or more polymeric components (constituents) of native ECM. ECM preparations can be considered to be “decellularized” or “acellular”, meaning the cells have been removed from the source tissue or culture.

In some embodiments, the ECM is isolated from a vertebrate animal, for example, from a mammalian vertebrate animal including, but not limited to, human, monkey, pig, cow, sheep, etc. The ECM may be derived from any organ or tissue, including without limitation, urinary bladder, intestine, liver, heart, esophagus, spleen, stomach and dermis. In specific non-limiting examples, the extracellular matrix is isolated from esophageal tissue, urinary bladder, small intestinal submucosa, dermis, umbilical cord, pericardium, cardiac tissue, or skeletal muscle. The ECM can comprise any portion or tissue obtained from an organ, including, for example and without limitation, submucosa, epithelial basement membrane, tunica propria, etc. In one non-limiting embodiment, the ECM is isolated from urinary bladder.

The scaffold can include pharmaceutical agents of interest. In some embodiments, the scaffold provides sustained release of one or more pharmaceutical agents. In more embodiments, the pharmaceutical agent is a molecule that inhibits de-differentiation (or epithelial to mesenchymal transition) of RPE cells or inhibit formation of drusen-deposits underneath RPE cells or suppresses reactive oxygen species in RPE cells. In some non-limiting examples, the inhibitor of RPE cell de-differentiation can be L,745,870 (3-([4-(4-chlorophenyl)piperazin-1-yl]methyl)-1H-pyrrolo[2,3-b]pyridine) or a dopamine receptor inhibitor. The pharmaceutical agent can be metformin, a Nox4 inhibitor, a reactive oxygen inhibitor aminocaproic acid, Riluzole, or a NK-κβ inhibitor. In a specific non-limiting example, the pharmaceutical agent is L,745,870. In another specific non-limiting example, the pharmaceutical agent is metformin. In a further non-limiting example, the pharmaceutical agent is a Nox4 inhibitor (VAS2870, GKT 137831 or GLX7013114). In a further non-limiting example, the pharmaceutical agent is a reactive oxygen species inhibitor (GKT 137831 or GLX7013114 or N-acetylcysteine).

The scaffold can be sterilized prior to seeding retinal pigment epithelial cells on the scaffold. In some embodiments, gamma irradiation is utilized to sterilize the scaffold. In other embodiments, an electron beam (ebeam) is used to sterilize the scaffold. Exemplary methods are disclosed, for example, in Bruyas et al., Tissue Eng. Part A, doi: 10.1089/ten.TEA.2018.0130 (Sep. 20, 2018) and Proffen et al., J. Orthop. Res. 33(7) 1015-1023 (2015)).

In some embodiments, retinal pigment epithelial cells are seeded onto the PLGA scaffold at about 125,000 to about 500,000 cells per 12 mm diameter of PLGA scaffold, such as about 150,000 cells per 12 mm diameter of PLGA scaffold, about 200,000 cells per 12 mm diameter of PLGA scaffold, about 250,000 cells per 12 mm diameter of PLGA scaffold, about 300,000 cells per 12 mm diameter of PLGA scaffold, about 350,000 cells per 12 mm diameter of PLGA scaffold, about 400,000 cells per 12 mm diameter of PLGA scaffold or about 450,000 cells per 12 mm diameter of PLGA scaffold.

In some embodiments, mature RPE cells are developed into functional RPE cell monolayers that behave as intact RPE tissue by continued culture in the RPE-MM with additional chemicals or small molecules that promote RPE maturation on the scaffold. In some embodiments, these small molecules are primary cilium inducers such as prostaglandin E2 (PGE2) or aphidicolin. The PGE2 can be added to the medium at a concentration of about 25 μM to about 250 μM, such as about 50 μM to about 100 μM. Alternatively, the RPE-MM can comprise canonical WNT pathway inhibitors. Exemplary canonical WNT pathway inhibitors are N-(6-Methyl-2-benzothiazolyl)-2-[(3,4,6,7-tetrahydro-4-oxo-3-phenylthieno[3,2-d]pyrimidin-2-yl)thio]-acetamide (IWP2) or 4-(1,3,3a,4,7,7a-Hexahydro-1,3-dioxo-4,7-methano-2H-isoindol-2-yl)-N-8-quinolinyl-Benzamide (endo-IWR1).

In some embodiments, for the continued maturation of the RPE cells, the cells can be dissociated in a cell dissociated enzyme such as TRYPLE™ and reseeded onto the scaffold, such as in a specialized SNAPWELL™ design, for at least about one to two weeks in RPE-MM with a MEK inhibitor such as PD0325901. Alternatively, the RPE-MM can comprise a bFGF inhibitor instead of the MEK inhibitor. Suitable methods for culturing RPE cells on the degradable scaffold are taught and described in PCT Publication No. WO 2014/121077, which is incorporated herein by reference in its entirety. Briefly, the main components of this method are a CORNING® COSTAR® SNAPWELL™ plate, a bioinert O-ring, and a biodegradable scaffold, such as any of the PLGA scaffold disclosed above. SNAPWELL™ plates provide the structure and platform for biodegradable scaffolds. The microporous membrane that creates an apical and basal side provides support to the scaffold as well as isolating the distinct sides of the polarized layer of cells. The ability of the SNAPWELL™ insert to detach the membrane allows the support ring of the insert to be used an anchor for the scaffold (see below). The resulting differentiated, polarized, and confluent monolayers of functional RPE cells on the scaffold can then be isolated and used as the tissue replacement implant.

In yet other embodiments the RPE cells on the scaffold have a resting potential of about −50 to about −60 mV, and a fluid transport rate of about 5 to about 10 μl cm⁻²h⁻¹. In additional embodiments, the RPE cells express MITF, PAX6, LHX2, TFEC, CDH1, CDH3, CLDN10, CLDN16, CLDN19, BEST1, TIMP3, TRPM1, TRPM3, TTR, VEGFA, CSPG5, DCT, TYRP1, TYR, SILV, SIL1, MLANA, RAB27A, OCA2, GPR143, GPNMB, MYO6, MYRIP, RPE65, RBP1, RBP4, RDH5, RDH11, RLBP1, MERTK, ALDH1A3, FBLN1, SLC16A1, KCNV2, KCNJ13, and CFTR, express miR204 and miR211, have a resting potential of about −50 to about −60 mV and have a fluid transport rate of about 5 to about 10 μl cm⁻²h⁻¹. In other embodiments, the RPE cells have a transepitelial resistance of greater than 1502*cm², such as greater than 200Ω*cm². In further embodiments, the RPE cells have a transepitelial resistance of 200Ω*cm² to 500Ω*cm², such as a transepitelial resistance of 200Ω*cm² to 400Ω*cm².

In one non-limiting example, a method for producing the tissue replacement implant includes: a) obtaining PLGA coated with vitronectin, wherein the PLGA scaffold comprises fibers that forming mesh structure and wherein the PLGA scaffold has an upper surface and a lower surface, wherein the PLGA scaffold is about 20- about 30 microns in thickness, has a DL-lactide/glycotide ratio of about 1:1, an average pore size of less than about 1 microns, and a fiber diameter of about 150 to about 650 nm; b) treating the scaffold with heat to fuse fibers of the scaffold at the junctions of fiber intersections within the PLGA scaffold to increase mechanical strength of the PLGA scaffold & to reduce pore size; c) seeding retinal pigment epithelial cells onto the PLGA scaffold at about 125,000 to about 500,000 cells per 12 mm diameter of PLGA scaffold; and d) culturing the retinal pigment epithelial cells on the PLGA scaffold in a tissue culture medium in vitro, with medium present on both the upper surface and the lower surface of the PLGA scaffold, for a time that is sufficient for i) polarization of the retinal pigment epithelial cells and ii) bulk degradation of the PLGA scaffold.

### Inhibitors of Use in Preparing a Tissue Replacement Implant

Disclosed below are inhibitors that are of use in preparing RPE cells and the disclosed tissue implants.

### 1. WNT Pathway Inhibitors

WNT is a family of highly conserved secreted signaling molecules that regulate cell-to-cell interactions and are related to the Drosophila segment polarity gene, wingless. In humans, the WNT family of genes encodes 38 to 43 kDa cysteine rich glycoproteins. The WNT proteins have a hydrophobic signal sequence, a conserved asparagine-linked oligosaccharide consensus sequence (see e.g., Shimizu et al Cell Growth Differ 8: 1349-1358 (1997)) and 22 conserved cysteine residues. Because of their ability to promote stabilization of cytoplasmic beta-catenin, WNT proteins can act as transcriptional activators and inhibit apoptosis. Overexpression of particular WNT proteins has been shown to be associated with certain cancers.

A WNT inhibitor herein refers to WNT inhibitors in general. Thus, a WNT inhibitor refers to any inhibitor of a member of the WNT family proteins including Wnt1, Wnt2, Wnt2b, Wnt3, Wnt4, Wnt5A, Wnt6, Wnt7A, Wnt7B, Wnt8A, Wnt9A, Wnt10a, Wnt11, and Wnt16. Certain embodiments of the present methods concern a WNT inhibitor in the differentiation medium. Examples of suitable WNT inhibitors, already known in the art, include N-(2-Aminoethyl)-5-chloroisoquinoline-8-sulphonamide dihydrochloride (CKI-7), N-(6-Methyl-2-benzothiazolyl)-2-[(3,4,6,7-tetrahydro-4-oxo-3-phenylthieno[3,2-d]pyrimidin-2-yl)thio]-acetamide (IWP2), N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (IWP4), 2-Phenoxybenzoic acid-[(5-methyl-2-furanyl)methylene]hydrazide (PNU 74654) 2,4-diamino-quinazoline, quercetin, 3,5,7,8-Tetrahydro-2-[4-(trifluoromethyl)phenyl]-4H-thiopyrano[4,3-d]pyrimidin-4-one (XAV939), 2,5-Dichloro-N-(2-methyl-4-nitrophenyl)benzenesulfonamide (FH 535), N-[4-[2-Ethyl-4-(3-methylphenyl)-5-thiazolyl]-2-pyridinyl]benzamide (TAK 715), Dickkopf-related protein one (DKK1), and Secreted frizzled-related protein (SFRP1) 1. In addition, inhibitors of WNT can include antibodies to, dominant negative variants of, and siRNA and antisense nucleic acids that suppress expression of WNT. Inhibition of WNT can also be achieved using RNA-mediated interference (RNAi).

### 2. BMP Pathway Inhibitors

Bone morphogenic proteins (BMPs) are multi-functional growth factors that belong to the transforming growth factor beta (TGFβ) superfamily. BMPs are considered to constitute a group of pivotal morphogenetic signals, orchestrating architecture through the body. The important functioning of BMP signals in physiology is emphasized by the multitude of roles for dysregulated BMP signaling in pathological processes.

BMP pathway inhibitors may include inhibitors of BMP signaling in general or inhibitors specific for BMP1, BMP2, BMP3, BMP4, BMP5, BMP6, BMP7, BMP8a, BMP8b, BMP10 or BMP15. Exemplary BMP inhibitors include 4-(6-(4-(piperazin-1-yl)phenyl)pyrazolo[1,5-a]pyrimidin-3-yl)quinoline hydrochloride (LDN193189), 6-[4-[2-(1-Piperidinyl)ethoxy]phenyl]-3-(4-pyridinyl)-pyrazolo[1,5-a]pyrimidine dihydrochloride (Dorsomorphin), 4-[6-[4-(1-Methylethoxy)phenyl]pyrazolo[1,5-a]pyrimidin-3-yl]-quinoline (DMH1), 4-[6-[4-[2-(4-Morpholinyl)ethoxy]phenyl]pyrazolo[1,5-a]pyrimidin-3-yl]quinoline (DMH-2), and 5-[6-(4-Methoxyphenyl)pyrazolo[1,5-a]pyrimidin-3-yl]quinoline (ML 347).

### 3. TGFβ Pathway Inhibitors

Transforming growth factor beta (TGFβ) is a secreted protein that controls proliferation, cellular differentiation, and other functions in most cells. It is a type of cytokine which plays a role in immunity, cancer, bronchial asthma, lung fibrosis, heart disease, diabetes, and multiple sclerosis. TGF-β exists in at least three isoforms called TGF-β1, TGF-β2 and TGF-β3. The TGF-β family is part of a superfamily of proteins known as the transforming growth factor beta superfamily, which includes inhibins, activin, anti-mullerian hormone, bone morphogenetic protein, decapentaplegic and Vg-1.

TGFβ pathway inhibitors may include any inhibitors of TGFβ signaling in general. For example, the TGFβ pathway inhibitor is 4-[4-(1,3-benzodioxol-5-yl)-5-(2-pyridinyl)-1H-imidazol-2-yl]benzamide (SB431542), 6-[2-(1,1-Dimethylethyl)-5-(6-methyl-2-pyridinyl)-1H-imidazol-4-yl]quinoxaline (SB525334), 2-(5-Benzo[1,3]dioxol-5-yl-2-ieri-butyl-3H-imidazol-4-yl)-6-methylpyridine hydrochloride hydrate (SB-505124), 4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, left-right determination factor (Lefty), 3-(6-Methyl-2-pyridinyl)-N-phenyl-4-(4-quinolinyl)-1H-pyrazole-1-carbothioamide (A 83-01), 4-[4-(2,3-Dihydro-1,4-benzodioxin-6-yl)-5-(2-pyridinyl)-1H-imidazol-2-yl]benzamide (D 4476), 4-[4-[3-(2-Pyridinyl)-1H-pyrazol-4-yl]-2-pyridinyl]-N-(tetrahydro-2H-pyran-4-yl)-benzamide (GW 788388), 4-[3-(2-Pyridinyl)-1H-pyrazol-4-yl]-quinoline (LY 364847), 4-[2-Fluoro-5-[3-(6-methyl-2-pyridinyl)-1H-pyrazol-4-yl]phenyl]-1H-pyrazole-1-ethanol (R 268712) or 2-(3-(6-Methylpyridine-2-yl)-1H-pyrazol-4-yl)-1,5-naphthyridine (RepSox).

### 4. MEK Inhibitors

A MEK inhibitor is a chemical or drug that inhibits the mitogen-activated protein kinase enzymes MEK1 or MEK2. They can be used to affect the MAPK/ERK pathway. For example, MEK inhibitors include N-[(2R)-2,3-Dihydroxypropoxy]-3,4-difluoro-2-[(2-fluoro-4-iodophenyl)amino]-benzamide (PD0325901), N-[3-[3-cyclopropyl-5-(2-fluoro-4-iodoanilino)-6,8-dimethyl-2,4,7-trioxopyrido[4,3-d]pyrimidin-1-yl]phenyl]acetamide (GSK1120212), 6-(4-bromo-2-fluoroanilino)-7-fluoro-N-(2-hydroxyethoxy)-3-methylbenzimidazole-5-carboxamide (MEK162), N-[3,4-difluoro-2-(2-fluoro-4-iodoanilino)-6-methoxyphenyl]-1-(2,3-dihydroxypropyl)cyclopropane-1-sulfonamide (RDEA119), and 6-(4-bromo-2-chloroanilino)-7-fluoro-N-(2-hydroxyethoxy)-3-methylbenzimidazole-5-carboxamide (AZD6244).

5. bFGF Inhibitors

Basic fibroblast growth factor (also known as bFGF, FGF2 or FGF-β) is a member of the fibroblast growth factor family. bFGF is present in basement membranes and in the subendothelial extracellular matrix of blood vessels. In addition, bFGF is a common component of human ESC culture medium in which it is necessary for the cells to remain in an undifferentiated state.

bFGF inhibitor herein refer to bFGF inhibitors in general. For example, bFGF inhibitors include, but are not limited to N-[2-[[4-(Diethylamino)butyl]amino-6-(3,5-dimethoxyphenyl)pyrido[2,3-d]pyrimidin-7-yl]-N′-(1,1-dimethylethyl)urea (PD173074), 2-(2-Amino-3-methoxyphenyl)-4H-1-benzopyran-4-one (PD 98059), 1-tert-Butyl-3-[6-(2,6-dichlorophenyl)-2-[[4-(diethylamino)butyl]amino]pyrido[2,3-d]pyrimidin-7-yl]urea (PD161570), 6-(2,6-Dichlorophenyl)-2-[[4-[2-(diethylamino)ethoxy]phenyl]amino]-8-methyl-pyrido[2,3-d]pyrimidin-7(8H)-one dihydrochloride hydrate (PD166285), N-[2-Amino-6-(3,5-dimethoxyphenyl)pyrido[2,3-d]pyrimidin-7-yl]-N′-(1,1-dimethylethyl)-urea (PD166866), and MK-2206.

### Use of the Tissue Replacement Implants and Methods of Treatment

Tissue replacement implants are provided that include a biodegradable scaffold and an effective amount of RPE cells. The tissue replacement implants described herein, and pharmaceutical compositions including these implants, can be used for the manufacture of a medicament to treat a condition in a patient in need thereof.

In some embodiments, the disclosed RPE cells are derived from iPSCs, and thus can be used to provide “personalized medicine” for patients with eye diseases. In some embodiments, cells obtained from patients, such as somatic cells or CD34+ cells, or umbilical cells, can be used to produce iPSC, which are then used to produce RPE cells. In some embodiments, the RPE cells (or the starting iPSC) can be genetically engineered to correct the disease-causing mutation, differentiated into RPE, and engineered to form a tissue implant. This tissue replacement implant can be used to replace the endogenous degenerated RPE of the same subject.

Alternatively, iPSCs can be generated from a healthy donor or from HLA homozygous “super-donors” or “universal” donor iPS cells and used to prepare the tissue implant. RPE cells can be treated in vitro with certain factors, such as pigment epithelium-derived factor (PEDF), transforming growth factor (TGF)-beta, and/or retinoic acid to generate an anti-inflammatory and immunosuppressive environment in vivo. These “superdonor” iPSC are commercially available, see the Cellular Dynamics International (for example, see the internet, globenewswire.com/news-release/2015/02/09/704392/10119161/en/Cellular-Dynamics-Manufactures-cGMP-HLA-Superdonor-Stem-Cell-Lines-to-Enable-Cell-Therapy-With-Genetic-Matching, Feb. 15, 2015).

The subject can be a human or veterinary subject. The RPE cells can be derived from a single subject, or several populations of RPE cells, such as 1, 2, 3, 4, or 5 types of RPE, each derived from a different subject, can be used in the implant.

Various eye conditions may be treated or prevented by the introduction of the tissue implant. The conditions include retinal diseases or disorders generally associated with retinal dysfunction or degradation, retinal injury, and/or loss of retinal pigment epithelium. The disclosed tissue replacement implants are of use for treating a retinal degenerative disease, retinal or retinal pigment epithelium dysfunction, retinal degradation, retinal or retinal pigment epithelial damage, such as damage caused by light, laser, inflammatory, infectious, radiation, neovascular or traumatic injury. The disclosed tissue replacement implants are also of use for treating loss of retinal pigment epithelium. The methods include locally administering the tissue replacement implant to the eye of the subject.

In some embodiments the retina degenerative disease is Stargardt's macular dystrophy, retinitis pigmentosa, age related macular degeneration, glaucoma, diabetic retinopathy, Lebers congenital amaurosis, late-onset retinal degeneration, hereditary macular or acquired retinal degeneration, Best disease, Sorsby's fundus dystrophy, retinal detachment, gyrate atrophy, traumatic eye injury, or choroideremia, pattern dystrophy. Additional conditions include retinal detachment, pattern dystrophy, and other dystrophies of the RPE. In a specific non-limiting example, the subject has age related macular degeneration. In certain embodiments, methods are provided for treating or preventing a condition characterized by retinal degeneration, comprising administering to a subject in need thereof the tissue replacement implant.

These methods can include selecting a subject with one or more of these conditions and administering the tissue replacement implant to treat the condition and/or ameliorate symptoms of the condition. The tissue replacement implant may also be transplanted together (co-transplantation) with other retinal cells, such as with photoreceptors. In some embodiments, the RPE cells in the tissue replacement implant are from the subject to be treated, and thus are autologous. In other embodiments, the RPE cells in the tissue replacement implant are produced from an MHC-matched donor or a universal donor. In another embodiment, the RPE cells in the tissue replacement implant are allogeneic.

The tissue replacement implant can be introduced to various target sites within a subject's eye. In some embodiments, the tissue replacement implant is introduced, such as by transplantation, to the subretinal space of the eye, which is the anatomical location of the RPE (between the photoreceptor outer segments and the choroids) in mammals. Exemplary methods are disclosed, for example, in PCT Publication No. WO 2018/089521, incorporated herein by reference. In some embodiments, the tissue replacement implant is introduced in the outer retina, retinal periphery, macula, or peri-macular regions, or within a choroid. In addition, dependent upon migratory ability and/or positive paracrine effects of the cells, introduction into additional ocular compartments can be considered, such as the vitreal space, the inner or outer retina, the retinal periphery and within the choroids.

The size of the tissue replacement implant to be transplanted may be generally determined by comparing the clinical assessment of the size of the region of retinal pathology present in a particular patient, with the constraints imposed by surgical feasibility of delivering an implant of a particular size. For example, in degenerations involving the central retina (e.g. age-related macular degeneration), a circular implant of from about 1.0-2.5 mm diameter (e.g., of about 1.5 mm diameter) that approximates the anatomic fovea will frequently be appropriate. In some cases, larger implants may be appropriate, maximally corresponding to the area of posterior retina lying between the temporal vascular arcades (histologic macula, clinical posterior pole) which is an ovoid area of approximately 6.0 mm (vertical)×7.5 mm (horizontal) centered on the fovea or positioned in the extra-foveal region. In some instances, it may likewise be appropriate to fashion a polymer scaffold of smaller dimension, as small as about 0.5 mm, to be placed in an area of circumscribed pathology. In addition, it may be of interest to custom fashion implants of irregular shape to suit the patient, for instance to cover areas of pathology while avoiding areas of residual high vision.

The tissue replacement implant can be introduced by various techniques known in the art. Methods for performing transplants are disclosed in, for example, in U.S. Pat. Nos. 5,962,027, 6,045,791, and 5,941,250; Biochem Biophys Res Commun Feb. 24, 2000; 268(3): 842-6; and Ophthalmic Surg February 1991; 22(2): 102-8). Methods for performing corneal transplants are described in, for example, U.S. Pat. No. 5,755,785; Curr Opin Opthalmol August 1992; 3 (4): 473-81; Ophthalmic Surg Lasers April 1998; 29 (4): 305-8; and Ophthalmology April 2000; 107 (4): 719-24. In some embodiments, transplantation is performed via pars pana vitrectomy surgery followed by delivery of the tissue replacement implant through a small retinal opening into the sub-retinal space. Alternatively, the tissue replacement implant can be delivered into the subretinal space via a trans-scleral, trans-choroidal approach. In addition, direct trans-scleral insertion to the anterior retinal periphery in proximity to the ciliary body can be performed.

The tissue replacement implant can also be transplanted together (co-transplantation) with other cells, such as photoreceptors.

In some embodiments, the methods include administering an immunosuppressive agent that reduces an immune response, for example, by downregulating the response of inflammatory cells or by inducing apoptosis of inflammatory cells. In other embodiments, the method includes administering a therapeutically effective amount of a neuroprotective agent that promotes survival and/or reduces degeneration of retinal neurons. In yet other embodiments, the method can include administering a therapeutically effective amount of an agent to inhibit unwanted angiogenesis, for example, to counteract the choroidal new vessel (CNV) growth under the fovea in AMD patients. An exemplary therapeutic agent can reduce activity of vascular endothelial growth factor (VEGF), for example, by binding to the receptor site of active forms of VEGF and preventing interaction of VEGF with its receptors. A therapeutically effective amount of and that suppresses the expression of VEGF by inhibiting pathways leading to VEGF secretion, such as STAT3, NF-kB, HIF-1α. Other drugs can prevent atrophy of RPE cells by targeting complement pathway, autophagy, or NF-kB pathways.

In further embodiment, the method includes administering to the subject a therapeutically effective amount of Ciliary Neurotrophic Factor (CNTF), Brain-Derived Neurotrophic Factor (BDNF), or Pigment Epithelial Derived Factor (PEDF), which can be used, for example, to promote development or function of neurons such as photoreceptor cells. Other exemplary, non-limiting embodiments include administering to the subject a therapeutically effective amount of thrombospondin 1, an anti-inflammatory cytokine (for example, interleukin (IL)-lra, IL-6, Fas ligand or tumor growth factor (TGF)-beta, a neurotrophic/neuroprotective growth factor such as, but not limited to, glial cell line-derived growth factor. brain-derived neurotrophic factor, nerve growth factor, neurotrophin-3, -4/5, -6, and vitamin E. Such agents may be provided singly or in combination.

The disclosure is illustrated by the following non-limiting Examples.

### EXAMPLES

Good Manufacturing Practice (GMP)/clinical-grade manufacturing was used to produce an AMD-patient specific iPSC-derived RPE (iRPE)-patch using a biodegradable scaffold. These patches were tested in two different animal models. The autologous iRPE-patch avoids immune-rejection by patient's cells and since these iRPE cells do not show any cellular phenotypes of AMD, can increase autologous iRPE-patch survival and integration in patients' eyes. This approach to transplant the patch at the borders of dry AMD lesion rescues photoreceptors in the transition zone, where RPE has atrophied and the photoreceptors are still alive (Bird, et al., JAMA Ophthalmol 132, 338-345 (2014)). Furthermore, these patches can be generated with major histocompatibility (MHC) matched RPE cells, or with heterologous RPE cells. These patches can include RPE cells from one subject, or from multiple subjects.

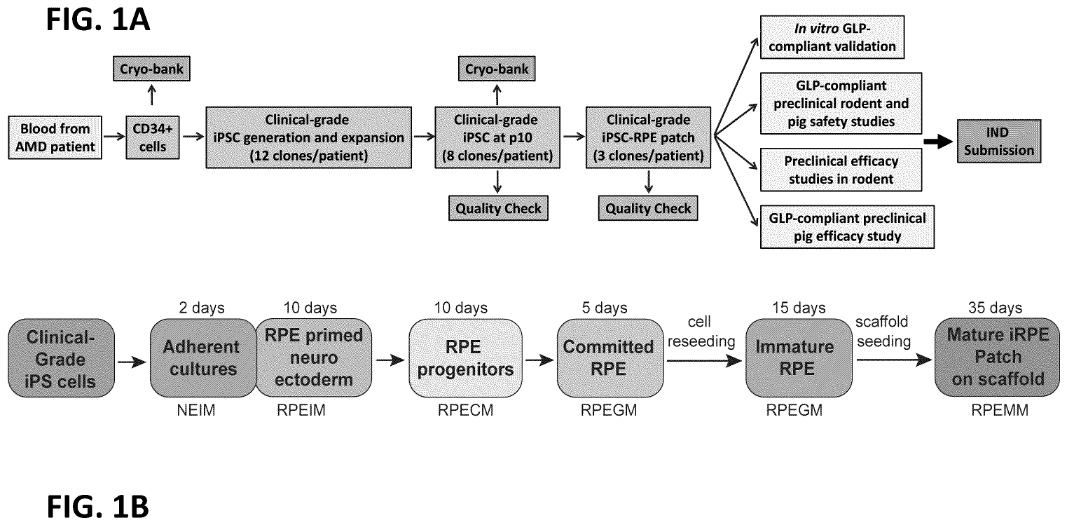

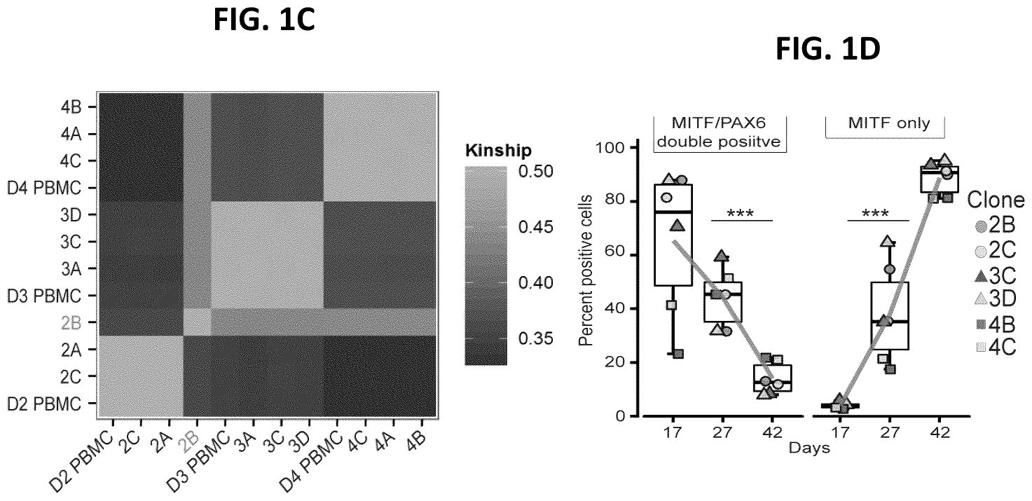

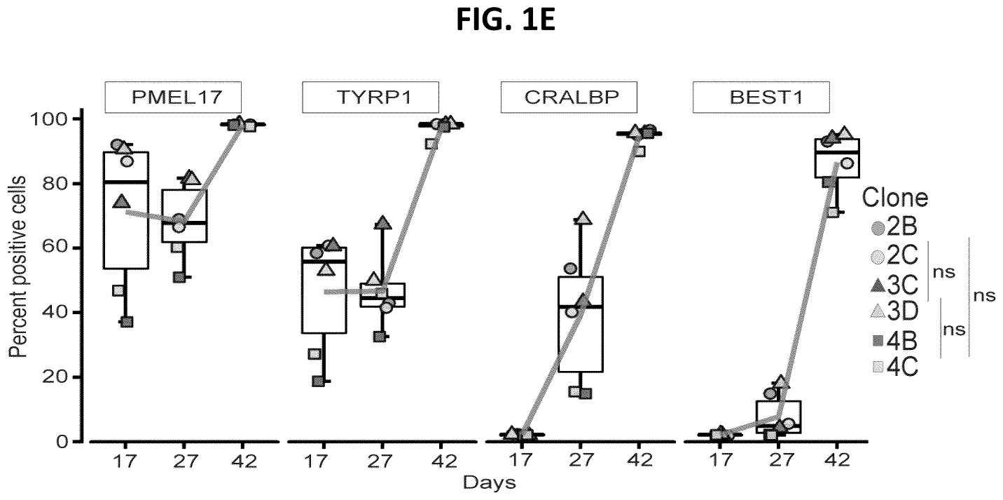

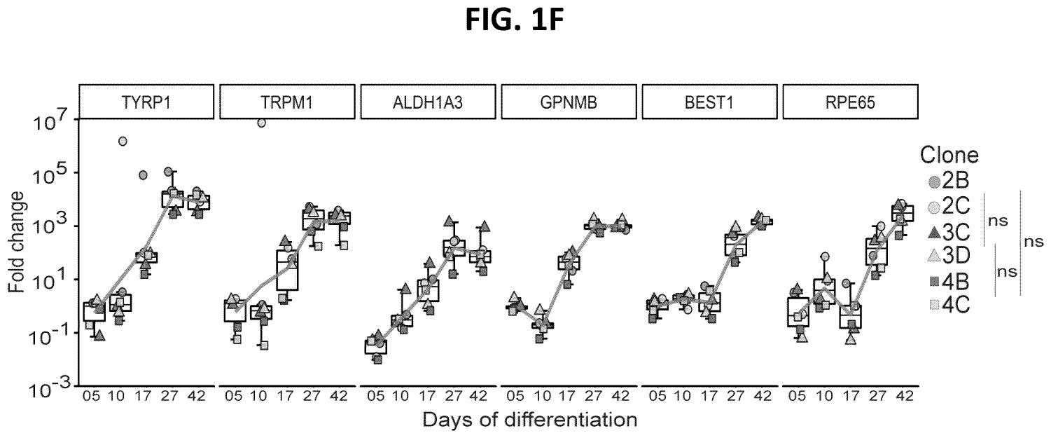

In the studies disclosed herein, the use of CD34+ cells isolated from the peripheral blood of AMD patients allowed development of oncogenic mutation-free clinical-grade iPSC banks. These iPSCs banks were used to develop a clinical-grade RPE differentiation process that is more efficient and reproducible as compared to the research-grade differentiation. Evidence is provided that iRPE-patches derived from three AMD patients do not show any cellular phenotypes of the disease, and mature and function to a similar extent. Additionally, it was demonstrated that seeding iRPE cells on a biodegradable-substrate significantly improved integration of the RPE monolayer as compared to transplanted cell suspension in immunocompromised rats, in rats with RPE-dysfunction associated retinal degeneration, and in a laser-induced RPE-injury pig model where the clinical dose of the iRPE-patch was tested. These experiments confirm the effectiveness of these tissue implants, and provide a complete workflow for performing pre-clinical studies to support an autologous iPSC-based phase I clinical trial for macular degeneration and for other disease indications (FIG. 1A).

### Example 1

### Materials and Methods

Research and clinical-grade iPSC derivation, maintenance, and RPE differentiation: Reporter iPSC line expressing GFP under the control of TYROSINASE enhancer and constitutive RFP was previously published (Maruotti et al., Proc Natl Acad Sci USA 112, 10950-10955 (2015)) and used to optimize the research-grade differentiation protocol. iPSCs were cultured on MEFs for 4 days before using for differentiation. To make cell aggregates, iPSCs were treated with Collagenase for 20 mins. After collagenase was aspirated, NEIM (DMEM/F12, KOSR, supplemented with N2, B27, LDN-193189 10 uM, SB431452 10 nM, CKI-7 hydrochloride 0.5 uM, and IGF-1 1 ng/ml) was added to the wells (1 ml/well) and cell scarper was used to scrape the colonies. Cell aggregates were grown in 10 cm² low attachment corning dishes in NEIM medium for 48 hrs. After 48 hours, floating cell aggregates were collected and seeded in MATRIGEL® coated plates in RPEIM (DMEM/F12, KOSR, supplemented with N2, B27, LDN-193189 100 uM, SB431452 100 nM, CKI-7 hydrochloride 5 uM, and IGF-1 10 ng/ml, PD0325901 1 uM) and cultured for 3 weeks. After 3 weeks in RPEIM, cells were moved to RPECM (DMEM/F12, KOSR, supplemented with N2, B27, Nicotinamde 10 mM and Activin A 100 ng/ml) for 3 more weeks with regular medium change. Pigmented patches of immature RPE cells were collected through differential trypsinization and seeded on to transwells or T-25 flasks in RPEGM (MEM, Sigma; 1% N2 supplement, ThermoFisher; 1% Glutamine, ThermoFisher; 1% non-essential amino acids, ThermoFisher; 125 mg Taurine (Sigma)/500 ml; 10 μg Hydrocortisone (Sigma)/500 ml; 0.0065 μg Triiodo-thyronin (Sigma)/500 ml; 5% FBS, Sigma) (Maminishkis et al., C Invest Ophthalmol Vis Sci 47, 3612-3624 (2006)). Table 1 provides a list of all reagents used in the manufacturing process. Flow cytometry was performed to check GFP expression in the differentiating cells at various time points.

TABLE 1
Detailed list of clinical-grade reagents used in iPSC generation
and RPE differentiation

| | | | Use |
|---|---|---|---|
| Reagents | Vendor | Catalogue | description |
| Plasmid 149 | Aldevron | p149 | Reprograming |
| Plasmid 150 | Aldevron | p150 | Reprograming |
| ViCell concentration | Beckman | B78897 | Differentiation |
| control | Coulter | | |
| 8X106 Beads/mL | | | |
| CryoStor CS10 | BioLife Solutions | 210102 | Ficoll/ |
| | | | Reprograming/ |
| | | | Differentiation |
| TPO 50 ug | CellGenix | 1017-050 | Reprograming |
| triCitrasol | Citra Labs | PN6030 | Ficoll |
| Fetal Bovine | GE Healthcare | SH30071.03 | Differentiation |
| Serum (U.S.), | (Hyclone) | | |
| Characterized | | | |
| DNase I | Genentech | 50242-100- | Reprograming |
| (PULMOZYME) | | 04010039 | |
| (2 ug in 2 mL) | | | |
| Lymphocyte | Lonza/corning | 17-829E/25- | Ficoll |
| Separation Medium | | 072-C1 | |
| CD34 | Lonza | VPA-1003 | Reprograming |
| Nucleofector ® | | | |
| Solution | | | |
| CD34 | Lonza | VPA-1003 | Reprograming |
| Nucleofector ® | | | |
| Supplement | | | |
| DPBS without Ca++ | Lonza/Life | 17- | Ficoll/ |
| and Mg++ | Technology | 512F/ | Reprograming/ |
| | | 14190250 | Differentiation |
| Versene Solution | Thermo Fisher | 15040-066 | Reprograming/ |
| | | | Differentiation |
| Recombinant hLIF | Millipore | LIF1010 | Reprograming |
| (10 ug/ml) | | | |
| H-1152 (1 mg) | Millipore | 555550 | Differentiation |
| CliniMACS ® | Miltenyi Biotec | 700-25 | Reprograming |
| PBS/EDTA Buffer | | | |
| ClimiMACs | Miltenyi Biotec | 171-01 | Reprograming |
| CD34+ microbeads | | | |
| CliniMACS CD56 | Miltenyi Biotec | 130-019-401 | Differentiation |
| microbeads | | | |
| CliniMACS | Miltenyi Biotec | 130-019-201 | Differentiation |
| Anti-Biotin | | | |
| microbeads | | | |
| Mouse Anti-CD24 | Miltenyi Biotec | Custom made | Differentiation |
| (Biotin Conjugated) | | | |
| DMSO | Mylan | 67457-178-10 | Reprograming/ |
| | Institutional | | Differentiation |
| Fit-3 (50 ug) | R&D Systems | 308-GMP-050 | Reprograming |
| SCF (50 ug) | R&D Systems | 255-GMP-050 | Reprograming |
| IL-6 (50 ug) | R&D Systems | 206-GMP-050 | Reprograming |
| IL-3 (50 ug) | R&D Systems | 203-GMP-050 | Reprograming |
| Recombinant | R&D Systems | AFL 338 | Differentiation |
| Human Activin A, | | | |
| Animal-Free | | | |
| (60 ug, 645 ul/vial, | | | |
| 0.094 mg/ml) | | | |
| CUSTOM VIALED | | | |
| Recombinant | R&D Systems | 291-GMP- | Differentiation |
| Human IGF-I, | | 5.5ug | |
| Animal-Free | | | |
| (5.5 ug, 55 uL)- | | | |
| CUSTOM VIALED | | | |
| PGE2 (10 mg) | R&D Systems | 2296 | Differentiation |
| SB431542 | R&D Systems | 1614 | Differentiation |
| Hydrate (10 mg) | | | |
| Blebbistatin (5 mg) | Sigma Aldrich | B0560 | Differentiation |
| Ascorbic Acid (2 g) | Sigma Aldrich | PHR1008-2G | Differentiation |
| CKI-7 | Sigma Aldrich | C0742-5mg | Differentiation |
| Dihydrochloride | | | |
| (5 mg) | | | |
| Niacinamide (1 g) | Sigma Aldrich | PHR1033-1G | Differentiation |
| Taurine (1 g) | Sigma Aldrich | PHR1109-1G | Differentiation |
| Hydrocortisone | Sigma Aldrich | H-6909 | Differentiation |
| (50 uM) | | | |
| 3,3′,5-Triiodo- | Sigma Aldrich | T5516 | Differentiation |
| L-thyronine | | | |
| sodium salt (1 mg) | | | |
| Y-27632 (1 mg) | Sigma Aldrich | Y0503-1MG | Reprograming |
| Beta- | Sigma Aldrich | 07604 | Reprograming |
| Mercaptoethanol | | | |
| HA-100 (5 mg) | StemCell | 72482 | Reprograming |
| | Technologies | | |
| StemSpan ACF | StemCell | 09805 | Reprograming |
| | Technologies | | |
| CHIR99021 (2 mg) | Stemgent | 04-0004-02 | Reprograming |
| A-83-01 (2 mg) | Stemgent | 04-0014 | Reprograming |
| LDN-193189 | Stemgent | 04-0074-10 | Differentiation |
| (10 mg) | | | |
| PD325901 (2 mg) | Stemgent | 04-0006 | Differentiation |
| Retronectin (2.5 mg) | Takara | T202 | Reprograming |
| UltraPure 0.5M | Thermo Fisher | 15575-020 | Differentiation |
| EDTA, pH 8 | | | |
| CTS N2 Supplement | Thermo Fisher | A13707-01 | Reprograming/ |
| (100 X) | | | Differentiation |
| 100x Glutamax | Thermo Fisher | 35050-061 | Reprograming |
| E8 Basal medium | Thermo Fisher | A15169-01 | Reprograming/ |
| | | | Differentiation |
| E8 supplement | Thermo Fisher | A15170-01 | Reprograming |
| CTS Vitronectin | Thermo Fisher | A27940 | Reprograming/ |
| (VTN-N) | | | Differentiation |
| DMEM/F12, HEPES | Thermo Fisher | 11330-032 | Reprograming/ |
| | | | Differentiation |
| MEM Alpha, | Thermo Fisher | 12571-063 | Differentiation |
| nucleosides | | | |
| MEM non-essential | Thermo Fisher | 11140-050 | Differentiation |
| AA | | | |
| Sodium Pyruvate | Thermo Fisher | 11360-070 | Differentiation |
| (100 mM) | | | |
| CTS KnockOut ™ | Thermo Fisher | A1099201 | Differentiation |
| SR XenoFree | | | |
| B-27 Supplement | Thermo Fisher | 17504-044 | Differentiation |
| 50 X, Serum | | | |
| free | | | |
| CTS TrypLE | Thermo Fisher | A12859-01 | Reprograming/ |
| Select Enzyme | | | Differentiation |
| Water for injection | APP | NDC-63323- | Reprograming/ |
| (WFI) | Pharmaceuticals | 185-10 | Differentiation |
| CTS B-27 | Thermo Fisher | A14867-01 | Reprograming |
| Supplement, | | | |
| XenoFree | | | |
| Recombinant | Waisman | WC-FGF2-FP- | Reprograming |
| human FGF2 | Biomanufacturing | 003 | |
| (0.1 mg/ml) | | | |
| 10M NaOH | Sigma Aldrich | 72068 | Differentiation |
| ViCell Reagent Pack | Beckman Coulter | 383722 | Differentiation |

Certificate of Analysis for all reagents have been validated for all these reagents for our clinical-grade manufacturing processes.

For clinical-grade protocol, feeder free iPSCs clones were derived from CD34+ PBMC using a previously published report (Mack, et al., PLoS One 6, e27956 (2011)). Mini banks of up to eight clones were validated for pluripotency by flow cytometry, sterility (WuXiApp Tech, Marietta, GA), normal G-band karyotyping (Cell Line Genetics, Madison, WI), STR identity (Univ. Madison Clinics, Madison, WI), plasmid loss (Cellular Dynamics Inc., Madison, WI), and oncogene sequencing (Q2 Solutions, Morrisville, NC). iPSCs cells were then seeded on vitronectin (A14701S, ThermoFisher, Carlsbad, CA) coated surface in E8 Medium (A1517001, ThermoFisher, Carlsbad, CA). After 2 days, cells were transferred to RPEIM for 10 days and then to RPECM for another 10 days. On day 22, cells were switched to RPEGM, trypsinized at day27 and reseeded in RPEGM. On day 42, cells were reseeded on to scaffolds in RPEMM (MEM, Sigma; 1% N2 supplement, ThermoFisher; 1% Glutamine, ThermoFisher; 1% non-essential amino acids, ThermoFisher; 125 mg Taurine (Sigma)/500 ml; 10 ug Hydrocortisone (Sigma)/500 ml; 0.0065 ug Triiodo-thyronin (Sigma)/500 ml; 5% FBS, Sigma; 50 uM PGE2, R&D Biosystems).

Real time PCR: Total RNA was isolated using NucleoSpin RNA (#740955, Machery-Nagel) per protocol. RNA was quantified using an ND-1000 spectrophotometer (Nanodrop Technologies). cDNA synthesis and custom made 24 RPE gene array plates were purchased from Bio-rad. Sybr green based Qpcr was run on ViiA 7 Real-Time PCR System (Thermo Fisher Scientific) according to manufacturer's protocol. Each sample was run in at least 3 biological replicate and data was analyzed in R-based software.

Trans Epithelial Resistance: Electric intactness was measured using EVOM2 and EndOhm chamber (World Precison Instruments) and each clone was measured for its resistance for 3 biological replicates.

Hexagonality measurement methodology: Cells were fixed in 4% paraformaldehyde and stained for Anti-Zonula Occluden-1 (ZO1) conjugated to AlexaFluor 594. Whole trans-wells were mounted and 2 mm×4 mm×60 micrometer sections of each well were imaged at 20× with a Zeiss Axio Scan-1. Z-stacks were then maximum-intensity projected (MIP) and cells in the MIP were analyzed for their borders using a convolution neural network. Once cell borders had been identified a binary “mask” was created to measure cell morphological properties. How close RPE were to an ideal convex regular hexagon was measured using a novel metric known as “Hexagonality”. Briefly, Hexagonality was assessed by taking 10 times the average of two different ratios (Eq. 3). The two ratios are the hexagon-side-ratio (HSR) as defined by Eq. 1 and the hexagon-area-ratio (HAR) as defined by Eq. 2.

$$\frac{{P}_{Cell}}{{P}_{Hull}} * [ 1 - \vert 1 - \frac{{P}_{cell}}{6 * \sqrt{\frac{4 * {A}_{cell}}{6 * cot ( \frac{π}{6} )}}} \vert ] = HSR \qquad Eq . 1$$

$$\frac{{A}_{Cell}}{{A}_{Hull}} * [ 1 - \vert 1 - \frac{4 * {A}_{cell}}{6 * {( \frac{{P}_{cell}}{6} )}^{2} * cot ( \frac{π}{6} )} \vert ] = HAR \qquad Eq . 2$$

$$10 * ( \frac{HSR + HAR}{2} ) = Hexagonality \qquad Eq . 3$$

In the above P_{cell} is the perimeter of the cell, P_{Hull} is the perimeter of the convex hull surrounding the cell, A_{Cell} is the area of the cell, A_{Hull} is the area of the convex hull.

Phagocytosis of Photoreceptor Outer Segments: Phagocytic ability of iRPE cells was measured using a published protocol with slight modifications (55). Bovine photoreceptor outersegments (POS) (InVision Bio) were labeled with pH-rodo dye (ThermoFisher) as per manufacturer's protocol. Mature iRPE cells after 5 weeks of culture on transwells or PLGA scaffolds were fed at the concentration if 5 POS/1RPE cell for 4 hours at 37C. Cells were washed with DPBS 3 times and incubated in 0.25% trypsin for 20 minutes. Trypsinized cells were collected with 1 ml pipette and suspended in 15 ml tube containing 10 ml of RPE MM. 15 ml tubes with cells were centrifuged at the 400 g for 5 minutes, the cell pellet was washed 3 times in 10 ml of DPBS, resuspended in 10 ml DPBS and the cell suspension was passed through a 0.44 um cell strainer.

Cells samples were run through MACS Miltenyi Flow Cytometer. Live cells were selected as DAPI negative. A laser channel with an excitation at 586 nm and emission at 515 nm was used to determine fluorescence from uptaken pH-rodo labelled POS. Flow cytometry data was analyzed with flowjo software and the median fluorescence for channel Y1 positive population was calculated for the fed and unfed samples. The ratio of fed samples to unfed samples was calculated and plotted in a graph.

Lactic Acid Measurements: PLGA scaffolds were incubated under the same conditions used for culturing of iPSC-RPE-patches. Culture medium was changed every alternate day and incubated medium was collected in a 15 ml tube. Media from three consecutive medium changes was combined in one tube. The same procedure was followed for all of the six technical replicates. Collected medium was immediately frozen and stored at −80 C. Lactic acid measurements were performed. Since each technical replicate tube from three consecutive media collections contained 12 ml of medium, it was lyophilized to reduce the total volume. Lyophilized material was resuspended in 1 ml of 1×D-PBS. Lactic acid measurement was performed using a Lactate Gen. 2 machine with a measuring range of 0.2-15.5 mmol/L (1.8-140 mg/dL).

Statistical Analysis: All statistical analysis was performed using R-software and where applicable the Dunn.test package. Data was first assessed for normality by determining data skewness, kurtosis, and q-q plots. All gene expression, TER, shape metrics, and phagocytosis data were found to have skewness or kurtosis values outside of a −1 to 1 range and showed significant deviance in q-q plots and thus were treated as non-normal. Dunn's test was therefore used for reporting multiple pairwise comparisons after a Kruskal-Wallis test for stochastic dominance among k groups was performed. A Bonferroni-Dunn correction was used for all pair-wise comparisons and an adjusted alpha of 0.05 was used for significance. For principle component analysis data from phagocytosis, TER, and gene expression profiles across all days and clones was scaled from 0 to 1 using the total pooled data for each metric. PCA was performed and clustering was shown based on k-nearest neighbors.

Oncogene Coding Variant Analysis: Coding regions and near exonic positions across 223 oncogenes were deeply sequenced by Q² Solutions (Morissville, NC) for each iPSC clone and accompany PBMC donor. Variants labeled as potentially deleterious were detected using Tute Genomics in the Q² provided variant call file (VCF). Additionally, three different somatic callers (Mutect version 1.15, SomaticSniper version 1.0.5.0; and Strelka version 1.0.15) were used to compare each iPSC clone to the matched PBMC. This parallel analysis found no new mutations in exon or splice positions.

Animals: Castrated Yucatan minipigs (RRID: NSRRC_0012) and Yorkshire pigs (age: 5-11 months; weigh: 30-40 Kg), Crl:NIH-Foxn1^{rmu} immunocompromised and Royal College of Surgeon rats were used. All animals were fed according to the body weight.

### Surgical Delivery of Cells in Rodents:

A. Suspension: Post-natal day (P) 21-28, RCS rats (RRID: RGD_1358258) or adult immunocompromised rats, Crl:NIH-Foxn1^{rmu} Nude rats (RRID #RGD_2312499) were anesthetized with 2, 2, 2-Tribromoethanol (intraperitoneal; 230 mg/kg; Sigma) and eyes received topical 0.5% proparacaine HCl anesthesia. Pupils were dilated with 1% tropicamide and 2.5% phenylephrine HCl and the eye was slightly proposed. A small scleral/choroidal incision (˜1 mm) was made 2 mm posterior to the limbus in the dorso-temporal region using increasing gauge needle tips. A small lateral corneal puncture was made using a 30-gauge needle to limit increase of intraocular pressure and reduce efflux of cells following injection. Two microliters of suspension containing the total cell dose (100,000 cells) was delivered into the subretinal space of one eye using a fine glass pipette (internal diameter, 75-150 μm) inserted into the subretinal space. The conjunctiva was then repositioned over the scleral incision. All animals included in the study received injection of cells with a minimum pre and post dose cell viability of 90%. All RCS rats received daily intraperitoneal injections of dexamethasone (1.6 mg/Kg) for 2 weeks post cell transplantation to minimize a potential inflammatory response.

B. iRPE-patch: Placement of the iRPE-patch followed the same general procedure as with suspension injection with the following exceptions. Subretinal blebs were created using 2-3 i balanced salt solution (BSS+) and scleral incision was extended using an 18ga needle to accommodate insertion of a 1-mm round implant. Using a sterile trephine, a 1-mm punch of RPE-scaffold was extracted from the culture plate. Using ILM peel forceps, the 1-mm implant was grasped at the distal end to offer protection and stability to the scaffold during implantation. Forceps were gently inserted into the subretinal space in the orientation of RPE facing the photoreceptors.

Optokinetic Tracking: Optokinetic tracking (OKT) thresholds were measured using a virtual optomotor system (VOS; CerebralMechanics, Lethbridge, AB, Canada) that allows evaluation of both the left and right eyes independently. Thresholds were evaluated at P90, using methods described elsewhere (39, 56). A single principle operator evaluated thresholds that were confirmed by a second operator.

Immunosuppression: Tetracycline antibiotics Doxycline and Minocycline were used orally is doses of 5 mg/Kg twice a day. A loading intramuscular dose of metilprednisolone was used at doses of 5 mg/Kg, followed by similar daily oral single doses of prednisone. Rapamycin was used orally with a loading dose of 2 mg, followed by a 1 mg daily dose. Tacrolimus was used in oral doses of 0.5 mg/day.

Laser-injury Model: An IQ 532micropulse laser (Iridex, USA) with a TXCELL™ scanning laser delivery device is used to selectively damage the RPE using a Volk HR centralis contact lens (Volk Optical Inc. Mentor, OH) with 740 field of view, 1/08× magnification and laser spot magnification of 0.93×. Micropulse power sufficient to obtained a mild whitening of the lasered area (1000-1600 milliwatts), and exposure times of 330 milliseconds are used. With micropulse duty cycles of 1% (0.100 milliseconds pulse “on” and 9.900 milliseconds pulse “off”) and a spot size of 200 microns, 7×7 confluent grids are made to create a 49 mm² lesion.

Pig iRPE-patch Transplantation: Sterilization of the surgical area with povidone iodine, a temporal canthotomy, superior rectus traction and nictitating membrane retraction was performed to increase the surgical exposure area. A nasal peritomy was done to exposed sclera and 4 surgical ports (infusion, chandelier illumination, and two working ports) are created 3.5 mm from limbus using 25G valve trocar cannulas (Alcon surgical, Fort Worth USA). After vitrectomy and posterior vitreous detachment, a localized retinal detachment (RD) was done in the visual streak (laser area) using a 25G/38G cannula (MedOne Surgical Inc. Sarasota, FL) and scissors retinotomy was done at the base of the RD. A sclerotomy (2.3-2.5 mm) was done in the area of the nasal port to accommodate the transplantation tool. The tip of the tool was introduced through the retinotomy into the subretinal space were the iRPE scaffold was released with the help of the viscous fluid injector device of the vitrectomy system (Alcon surgical, Fort Worth USA). An ocular wound clamp closed the sclerotomy to maintain the intraocular pressure while performing fluid air exchange to flatten the detached area which is confirmed by intraoperative OCT. The sclerotomy was closed with nylon 8-0.

Optical Coherence Tomography and Fluorescein Angiography: After the animal was adequately anesthetized, a Jet-Electrode was placed on the eye with an appropriate amount of GenTeal Tears (Alcon Fort Worth TX, NDC 0078-429-47). OCT was preformed using Spectralis (Heidelberg Engineering) with a 550 degree lens. The region of interest (ROI) was placed in the center and both averaged single B-scan across ROI and volume OCT scans covers entire ROI was performed. Follow-up function of the instrument was used to allow for OCT scans at the same retinal location for each examine time point. Spectralis was also used for capturing fluorescein angiogram with intravenous injection of Sodium Fluorescein (SF, Akorn Inc, Lake Forest, IL). Early (first min) and late phase (15 min) angiograms are recorded.

Multi-focal Electroretinography (mfERG): mfERG was recorded using the RETImap system (Roland Consult, Brandenburg Germany). Briefly, a bipolar contact lens (The GoldLens Corneal Electrode, Doran Instruments, Inc; Littleton, MA) was placed on the eye. A ground electrode, Genuine Grass Platinum Subdermal Needle Electrode (F-E2-12, Natus Manufacturing Limited, Galaway Ireland) was placed under the skin of the chin of the animal. A minimum of 3 cycles were used for each recording and a total of 3 overlapping retinal regions were measured. In each recording the ROI was shifted slightly around the field of view to account for slight differences in optics. A low bandpass of 10 hz and a high bandpass of 300 hz was used. The 7 mfERG components determined from the MATLAB program are N1 (first major trough) and P1 (the following peak) amplitudes (nV/deg²), N1P1 (difference between N1 and P1 amplitudes), Scalar Product, AUC, and widths of N1 and P1 (time in msec at half the max amplitude of peak). The 7 mfERG components are normalized by subtracting the laser signal of the implant with the healthy region signals and dividing by the pre-laser signal. A linear mixed model (LME) and ANOVA were performed to determine if statistical differences between groups. The equation function used in the fitlme function was: Data˜Week+Group*Component+(1|Pig_Name).

Immunostaining and Histology: Tissue preparation for immunohistochemistry was done by placing the eye in 4% PFA for a maximum of 4 hours after enucleation. The surgery area was dissected out and placed in 10% sucrose/PBS overnight followed by 24 hours in 20% sucrose/PBS. The samples were then placed in a 2:1 OCT:20% sucrose solution and flash frozen in a cryostat mold and placed in the −80 freezer until sectioning on the cryostat could be performed. Cryostat sectioning was done at 10 μm sections with tissue section separated every 50 μm. Immunohistochemistry was performed in general as follows: 5% Natural Goat Serum (NGS) (Thermo Fisher Scientific, Grand Island NY; #31873) blocking solution for 2 hrs followed by primary antibody incubation overnight in 1% NHS at room temperature. Primary antibodies include: RPE65 (1:300, Abcam, Cambridge, MA; ab78036, and custom antibody from M. Redmond lab/NEI 1:400), Biotinylated Peanut Agglutinin (PNA) (1:300, Vector Laboratories, Burlingame CA; B-1075), STEM121 (1:300 Takara Clontech Mountain View CA; Y40410), STEM101 (1:300, Takara Clonetch, Mountain View, CA), Rhodopsin (1:10,00, Encor Biotechnology Inc. Gainesville FL, MCA-B630); Red/Green Opsin (1:300 Millipore Burlington MA; AB5405); Blue Opsin (1:300 Millipore Burlington MA; AB5407); iPSCs—OCT4 (#653704, Biolegend, RRID: AB_2562018), TRA 1-81 (#560124, BD Biosciences); and SSEA-4 (#560126, BD Biosciences); RPE progenitors—MITF (#X2397M, Exalpha), PAX6 (#PRB-278P, Covance); committed RPE—PMEL17 (#HMB45, Dako), TYRP1 (#NBP2-32901, Novus Biologicals), and mature RPE—BEST1 (#NB300-164, Novus Biologicals). Ezrin (#E8897, SigmaAldrich), Collagen IV (#ab6311, Abcam), ALDH1A3 (#ab80176, Santa Cruz Biotechnology). Following overnight incubation, the tissue samples were washed 3× in 1% NGS solution and secondary antibodies conjugated to fluorescent markers, Alexa 488, Alexa 555 and or Alexa 633 (Thermo Fisher Scientific, Grand Island NY), to the appropriate primary in 1% NGS solution at 1:300 dilution. The slides were then either imaged on the Zeiss 800 confocal microscope or the Zeiss Axio Scan Z1 slide scanner. The number of nuclei (DAPI) in the implant region (either empty scaffold or iRPE-Patch) was normalized to the corresponding healthy region on the same section. The same distance across the retina (˜400 μm) was used for the implant area and the corresponding healthy area to count. (Unpaired t-test p<0.05; N=4 for empty scaffold, N=5 for iRPE-Patch).

### Example 2

### Clinical-Grade Triphasic Protocol Efficiently and Reproducibly Generates AMD-iRPE Cells

iPSC-derived from AMD-patient skin fibroblasts under clinical-grade conditions attained chromosomal copy number changes, likely during the reprograming process (Mandai et al., N Engl J Med 376, 1038-1046 (2017)). However, skin fibroblasts are not considered an ideal starting source when deriving iPSCs from older patients (Kang et al., Cell Stem Cell 18, 625-636 (2016)). Therefore, a manufacturing workflow was developed for autologous iPSC-derived RPE-patch from patient CD34+ peripheral blood cells. A pipeline for generation, functional validation, and in vivo testing of clinical-grade AMD patient-specific iPSC-RPE patch (FIGS. 1A, B) was developed. Due to the progenitor and proliferative nature, CD34+ cells isolated from patients' peripheral blood could provide a good source for iPSC generation. Multiple iPSC clones were generated from CD34+ cells and skin fibroblasts of three advanced “dry” AMD patients (ages 85, 89, and 87 years) using a clinical-grade episomal reprograming protocol (Mack, et al., PLoS One 6, e27956 (2011)). CD34+ cell derived iPSC were genomically more stable than skin fibroblasts derived iPSC (Mandai et al., N Engl J Med 376, 1038-1046 (2017)). Passage 10 banks were successfully generated from 3 iPSC clones/patient (derived from CD34+ cells) (Table 2).

TABLE 2
GMP-grade AMD iPSC Working Bank validation. iPSC Working Banks at passage 10 were validated
for being sterile (free of bacteria, fungus, and mycoplasma); normal G-band karyotyping,
expression of pluripotency markers (SSEA4, TRA1-60, TRA1-81, and OCT4 positivity); percent
cells that have lost the reprograming plasmid; identity of iPSCs with patient material.

| | | | | | | % ve | % ve |
|---|---|---|---|---|---|---|---|
| | | Pheno-¶ | | Sterility/ | Karyotype/ | SSEA4 ¶ | TRA1-60 ¶ |
| Donor [symbol] | Age¶ [symbol] | type [symbol] | Clone [symbol] | mycoplasma [symbol] | Gender [symbol] | cells [symbol] | cells [symbol] |
| 2 [symbol] | 85 [symbol] | Bilateral | A [symbol] | UD [symbol] | Normal•46/XY [symbol] | 99.3 [symbol] | 99.03 [symbol] |
| | | GA [symbol] | B [symbol] | UD [symbol] | Normal•46/XY [symbol] | 99.50 [symbol] | 100 [symbol] |
| | | | C [symbol] | UD [symbol] | Normal•46/XY [symbol] | 99.56 [symbol] | 99.56 [symbol] |
| 3 [symbol] | 89 [symbol] | Bilateral | A [symbol] | UD [symbol] | Normal•46/XY [symbol] | 99.25 [symbol] | 99.97 [symbol] |
| | | GA [symbol] | C [symbol] | UD [symbol] | Normal•46/XY [symbol] | 99.24 [symbol] | 100 [symbol] |
| | | | D [symbol] | UD [symbol] | Normal•46/XY [symbol] | 99.25 [symbol] | 99.97 [symbol] |
| 4 [symbol] | 87 [symbol] | Bilateral | A [symbol] | UD [symbol] | Normal•46/XX [symbol] | 99.8 [symbol] | 100 [symbol] |
| | | GA [symbol] | B [symbol] | UD [symbol] | Normal•46/XX [symbol] | 99.38 [symbol] | 100 [symbol] |
| | | | C [symbol] | UD [symbol] | Normal•46/XX [symbol] | 99.8 [symbol] | 100 [symbol] |

| | | | | | Onco-¶ |
|---|---|---|---|---|---|
| | | | %•Cells | STR•analysis¶ | gene• |
| | %•ve• | %•ve• | with | PBMC = iPSC¶ | sequence• |
| | TRA1-81•¶ | OCT4•¶ | plasmid | (patient• | (patient |
| Donor [symbol] | cells [symbol] | cells [symbol] | loss [symbol] | match) [symbol] | match) [symbol] |
| 2 [symbol] | 99.80 [symbol] | 99.94 [symbol] | 100 [symbol] | matched [symbol] | matched [symbol] |
| | 100 [symbol] | 100 [symbol] | 100 [symbol] | matched [symbol] | Several•¶ |
| | | | | | mis-match [symbol] |
| | 100 [symbol] | 99 [symbol] | 100 [symbol] | matched [symbol] | ••matched [symbol] |
| 3 [symbol] | 100.00 [symbol] | 99.96 [symbol] | 100 [symbol] | matched [symbol] | matched [symbol] |
| | 99.95 [symbol] | 100 [symbol] | 100 [symbol] | matched [symbol] | matched [symbol] |
| | 100.00 [symbol] | 99.96 [symbol] | 100 [symbol] | matched [symbol] | matched [symbol] |
| 4 [symbol] | 100 [symbol] | 99.96 [symbol] | 100 [symbol] | matched [symbol] | matched [symbol] |
| | 100 [symbol] | 99.96 [symbol] | 100 [symbol] | matched [symbol] | matched [symbol] |
| | 100 [symbol] | 99.96 [symbol] | 100 [symbol] | matched [symbol] | matched [symbol] |

¶
UD = undetectable.
Sterility was tested at WuXi AppTec (Mariette, GA);
G-band Karyotyping and STR analysis was performed at Cell Line Genetics (Madison, WI);
Plasmid Loss was detected using a fluidigm single cell qPCR assay at Cellular Dynamics International, Inc. (Madison, WI) [symbol]
indicates data missing or illegible when filed

These banks were further validated for: sterility; over 85% expression of SSEA4, TRA1-60, TRA1-81, and OCT4; normal G-band karyotyping; loss of reprograming plasmid; matched STR-identity; and matched oncogene sequence to the patient (Table 2). To determine if CD34+ cell derived iPSC acquired any sequence alterations during clinical-grade reprograming and expansion, coding regions of all 223 oncogenes were sequenced across the 9 iPSC clones. 8 iPSC clones matched their respective donor PBMCs with the exception of clone B from donor 2 (D2B) (FIG. 1C). However, none of the sequence changes in iPSC clone D2B were associated with any known cancerous phenotype (Q² Solutions, Morrisville, NC) and unlike Merkle et al, no mutations were seen in the coding region of the p53 tumor suppressor gene (Mandai et al., N Engl J Med 376, 1038-1046 (2017); Merkle et al., Nature 545, 229-233 (2017)). Thus, CD34+ cells are likely to produce iPSCs with minimal mutations during reprograming and can be used for autologous iPSC-based therapies. Using the above criteria, three validated iPSC clones per donor were selected for iRPE-patch manufacturing.

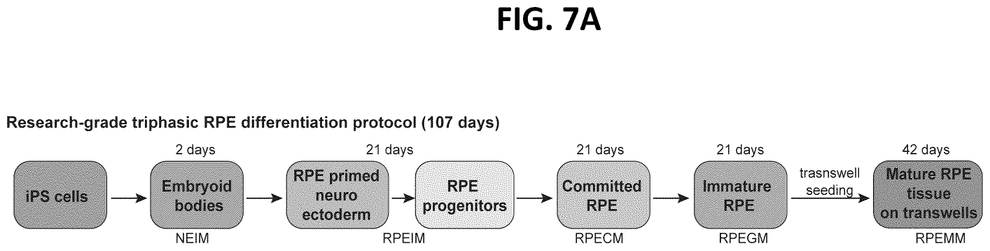

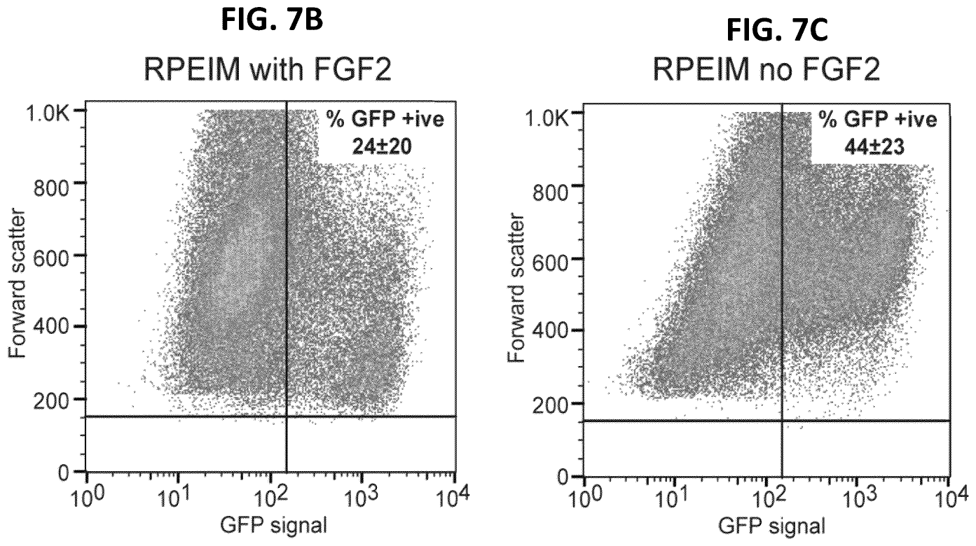

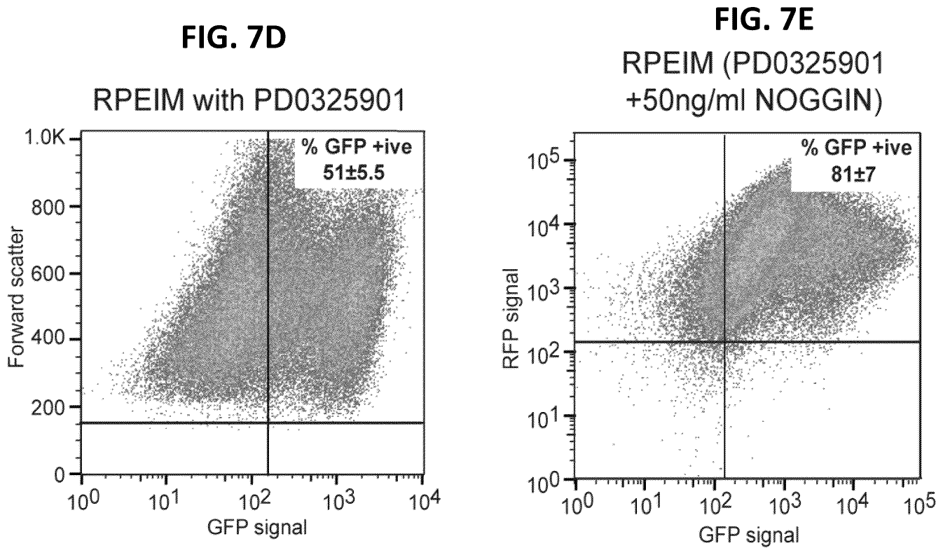

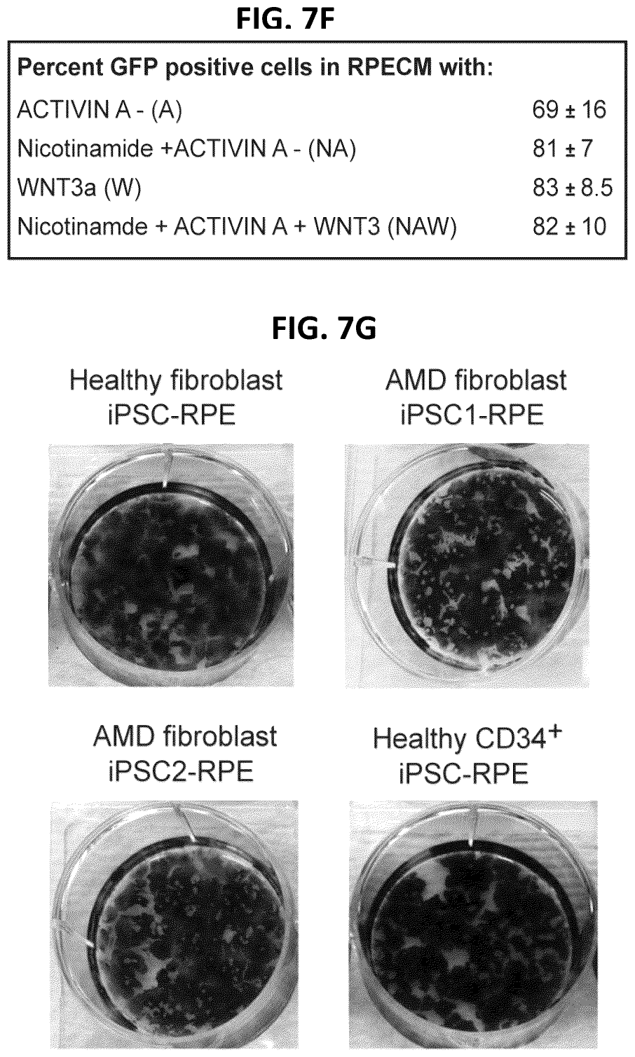

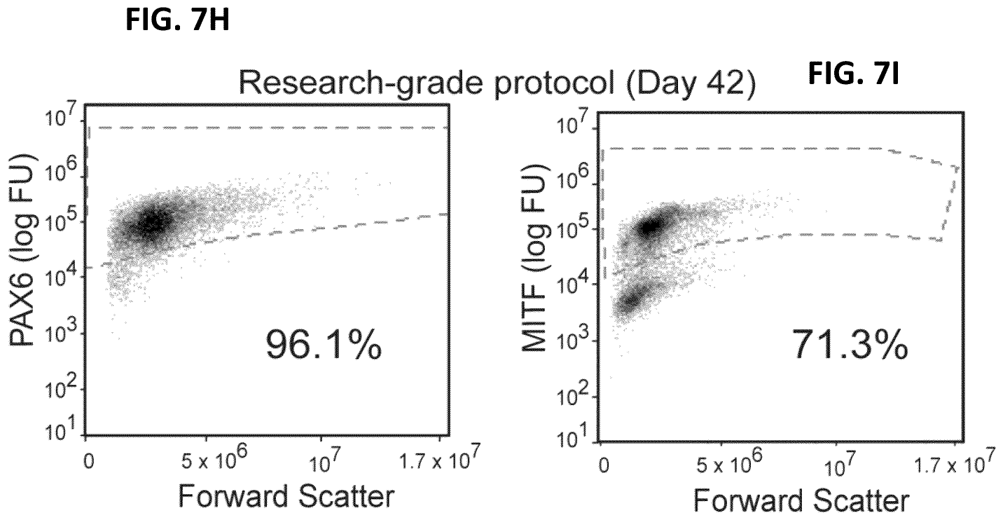

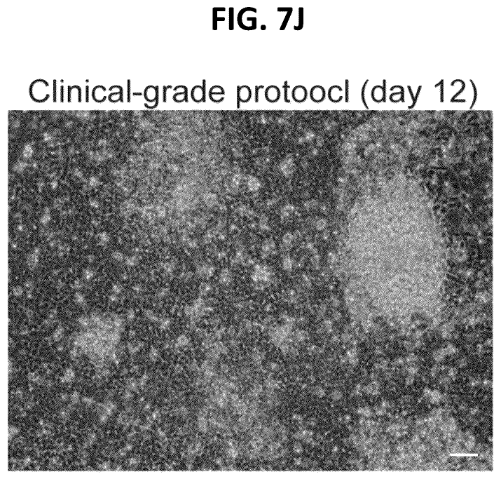

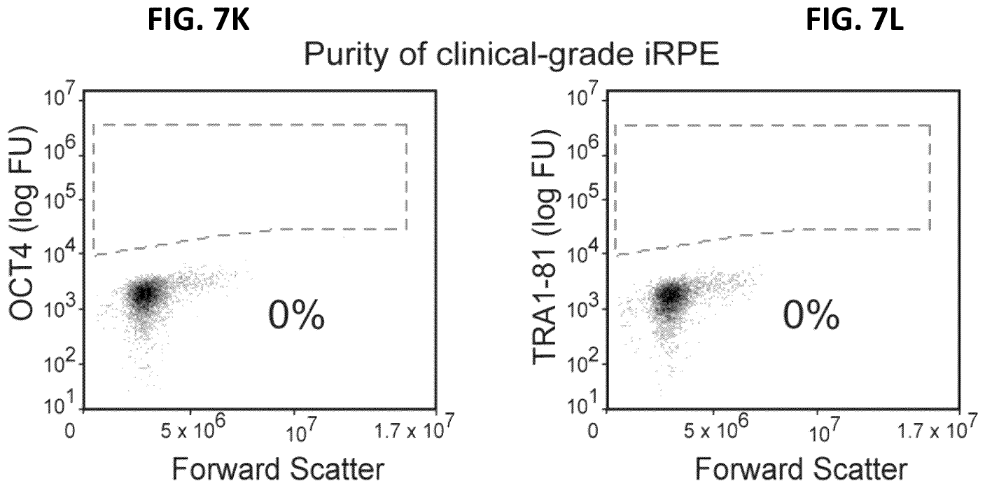

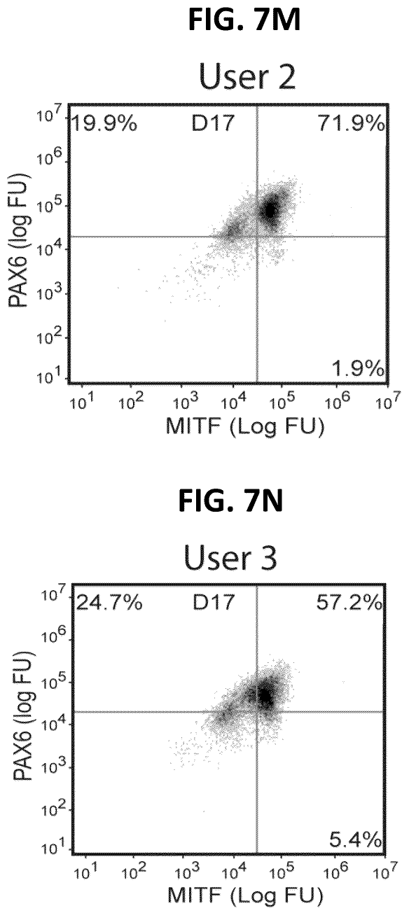

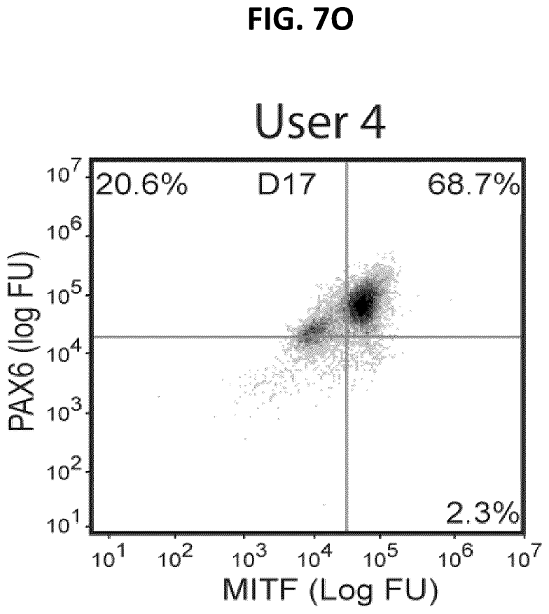

RPE differentiation can be induced in stem cells derived neuroectoderm cells by the activation of TGF or canonical WNT pathways (Idelson et al., Cell Stem Cell 5, 396-408 (2009); Leach, et al., Invest Ophthalmol Vis Sci 56, 1002-1013 (2015); Lamba, et al., Proc Natl Acad Sci USA 103, 12769-12774 (2006); Reh, et al., Methods Mol Biol 636, 139-153 (2010)). To further improve the efficiency and reproducibility of differentiation and to make iPSC-RPE manufacturing clinically-compatible, a triphasic differentiation protocol was optimized. (FIG. 7). The protocol comprised the following three outcomes: (1) dual SMAD inhibition promotes neuronal-fate and FGF pathway activation inhibits RPE phenotype (Fuhrmann, Curr Top Dev Biol 93, 61-84 (2010); Bharti et al., PLoS Genet 8, e1002757 (2012); Chambers et al., Nat Biotechnol 27, 275-280 (2009); Meyer et al., Proc Natl Acad Sci USA 106, 16698-16703 (2009)) and based on these observations, allow level of DUAL SMAD and FGF inhibition was combined to promote iPSCs into RPE-primed neuroectoderm cells and increased differentiation efficiency from 24% to 81% (FIGS. 7B-E); (2) activation of TGF or WNT pathways induced committed RPE-fate in these RPE-primed neuroectoderm at a much higher efficiency and reproducibility, as compared to the published protocols (see, Idelson et al., Cell Stem Cell 5, 396-408 (2009); Fuhrmann, Curr Top Dev Biol 93, 61-84 (2010); Carr et al., PLoS One 4, e8152 (2009)) (FIGS. 7F, G); and, (3) committed RPE were matured by inducing primary cilium with PGE2 treatment in cells to actively suppress canonical WNT pathway. Overall, this triphasic differentiation protocol improved iPSC-RPE differentiation efficiency, reproducibility, and generated mature RPE cells.

To test the reproducibility of the clinical-grade differentiation protocol across multiple iPSC clones and multiple patients, it was tested on iPSCs derived from all three AMD patients (two iPSC clones per patient). Two different starting conditions were compared, 3D cell aggregate v/s a 2D monolayer differentiation. The advantage of 2D monolayer protocol is that it improves the efficiency of differentiation and makes it user independent (FIGS. 1B, FIG. 7A, M). Despite starting with the same number of cells in 3D and 2D protocols, the 2D protocol was faster at inducing epithelial phenotype in cells. By day (D)12, cells displayed epithelial morphology prompting a shift to RPE Commitment Medium (RPECM). By D17, over 80% of cells in 4 out of 6 clones co-expressed PAX6/MITF and a small percent expressed only MITF, confirming the RPE-primed stage of neuroectoderm cells (FIG. 1D). As expected, by D27 the number of PAX6/MITF double positive RPE progenitors dropped to 30-40% and the number of MITF-only positive cells increased dramatically (20-60% across all six clones), suggesting a shift to the committed RPE population. By D42, over 80% cells across all six iPSC clones expressed MITF with a concomitant loss of PAX6/MITF double positive population, confirming the shift to immature RPE phenotype (FIG. 1D) (Idelson et al., Cell Stem Cell 5, 396-408 (2009)). In contrast, in the 3D protocol, most cells continued to be PAX6 positive by D42, suggesting an extended progenitor stage of cells (FIGS. 7H, I). Results with PAX6 and MITF expressing cells were further corroborated by the analysis of downstream and known targets of these genes (Idelson et al., Cell Stem Cell 5, 396-408 (2009); Fuhrmann, Curr Top Dev Biol 93, 61-84 (2010)). A large majority of cells across all six iPSC clones expressed RPE-progenitor markers PMEL17 (40-85%) and TYRP1(20-60% across six iPSC clones) at D17 and a negligible number of cells expressed RPE-maturity markers CRALBP and BEST1 (FIG. 1E). As the cells continue to mature, the expression of PMEL17 and TYRP1 stayed stable at D27 and increased to over 99% in all six iPSC clones by D42 after cell enrichment, confirming cell purity (FIG. 1E). In comparison, CRALBP and BEST1 positive cells continued to increase over time, reaching over 95% for CRALBP and over 70% for BEST1 across all six iPSC clones (FIG. 1E). The resultant RPE cells had no detectable iPSC (no OCT4 or TRA1-81+ cells; FIGS. 7K, 7L). Expression analysis for genes involved in RPE pigmentation (GPNMB and TYR), visual cycle (ALDH1A3, TRPM1, RPE65), and RPE maturation (RPE65 and BEST1) (Strunnikova et al., Human Molecular Genetics 19, 2468-2486 (2010)) confirmed that all six AMD-iPSC clones differentiated with similar efficiency and progressively attained maturity, underscoring reproducibility of the clinical-grade differentiation process (FIG. 1F). Furthermore, the manufacturing process reproducibility was confirmed across four different users (FIGS. 7M-7O). Overall, the 2D protocol was at least 40% faster and more efficient in iPSC-RPE differentiation as compared to the 3D research-grade protocol.

### Example 3

Biodegradable Scaffold Helps Clinical-Grade AMD-iRPE Cells to Functionally Mature into a Monolayer Tissue

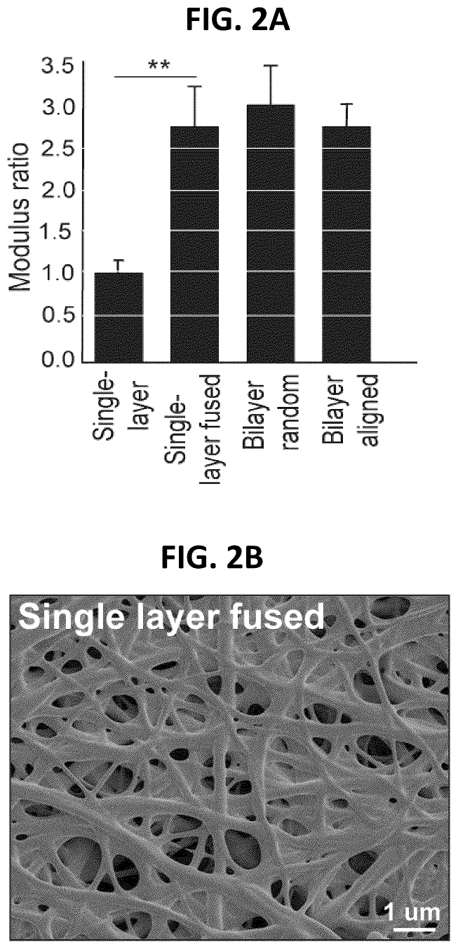

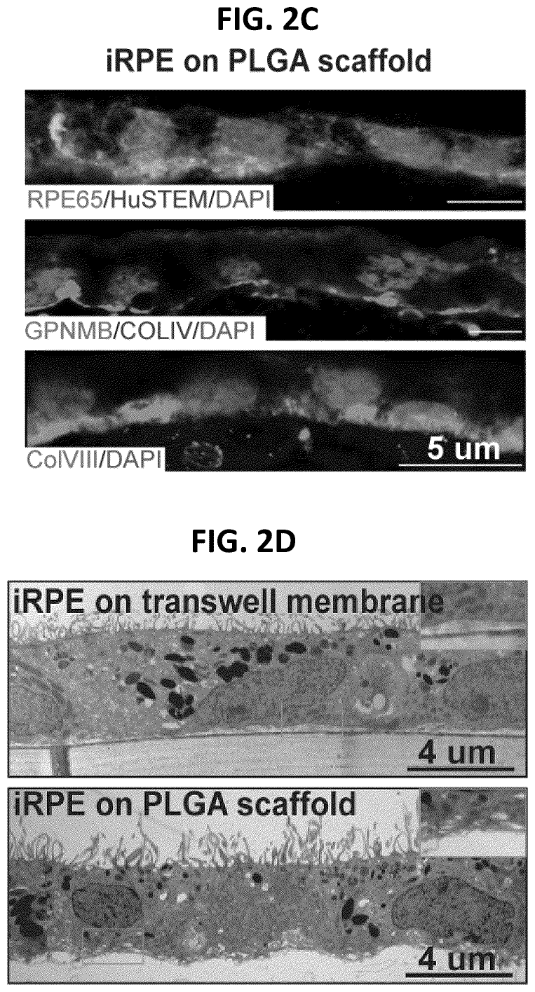

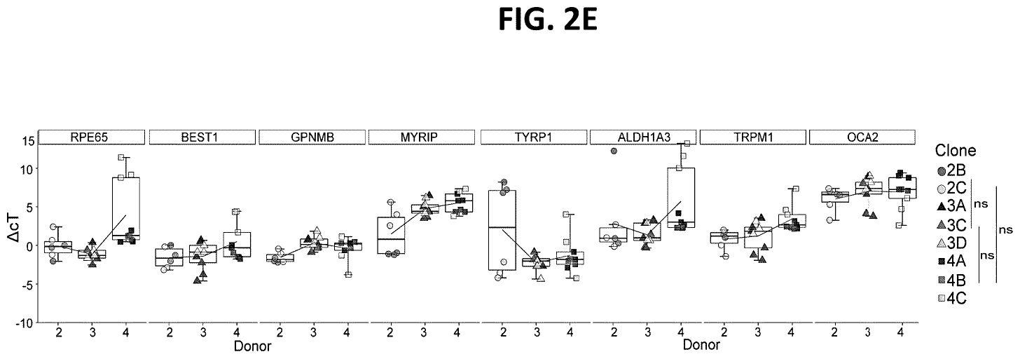

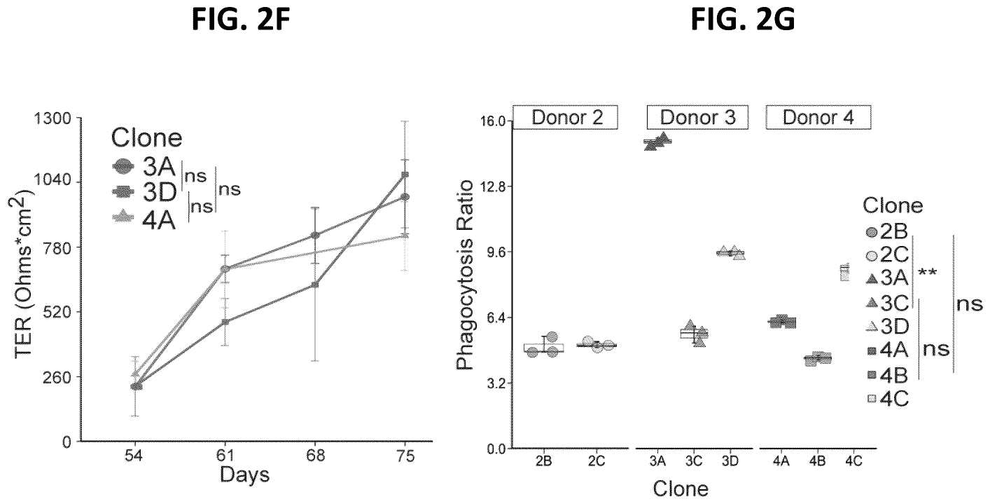

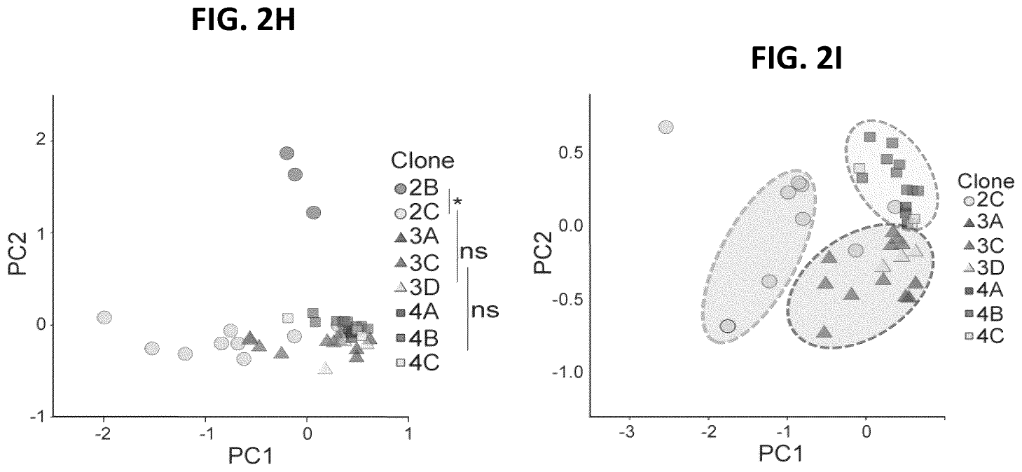

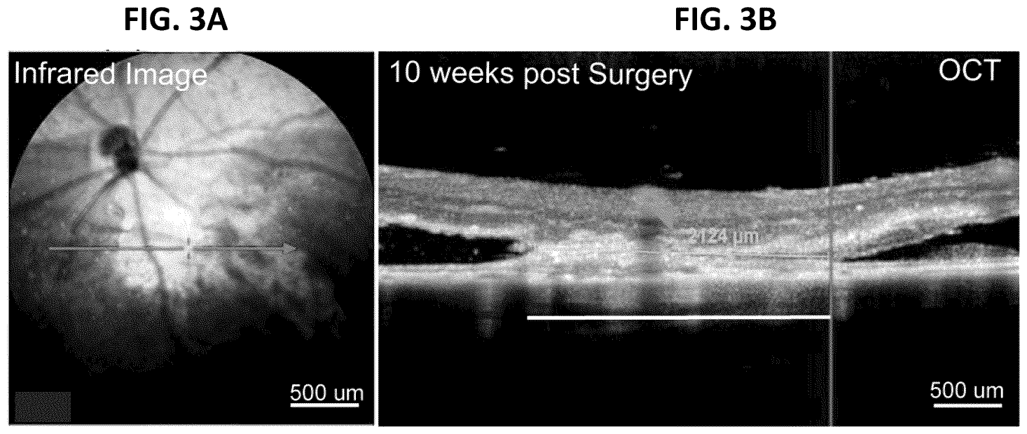

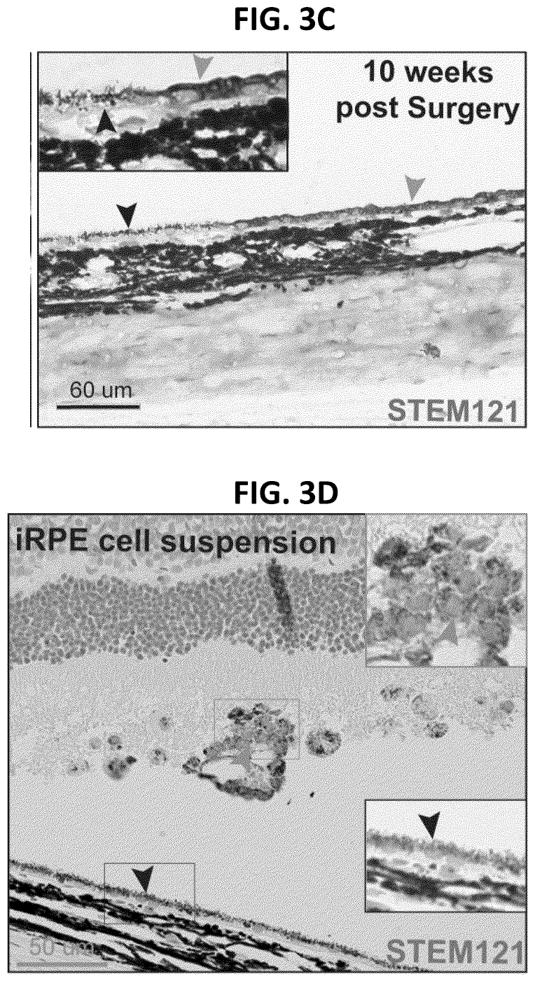

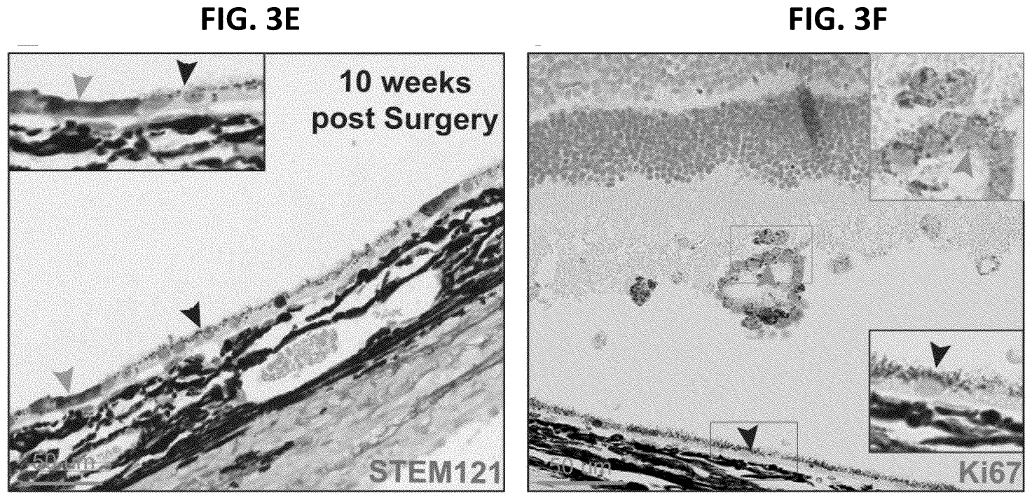

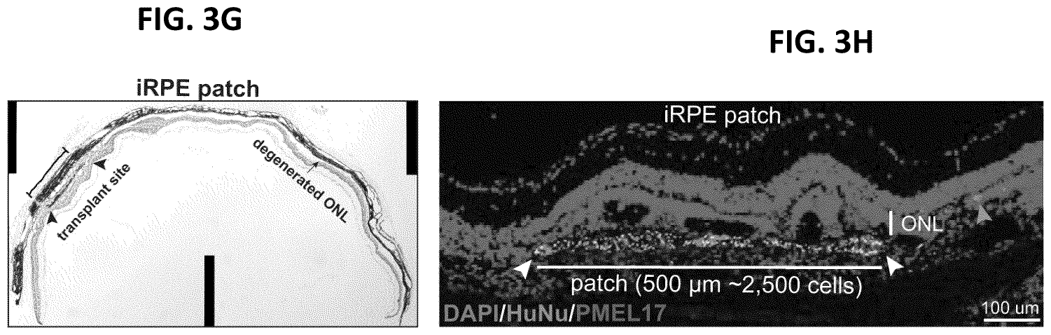

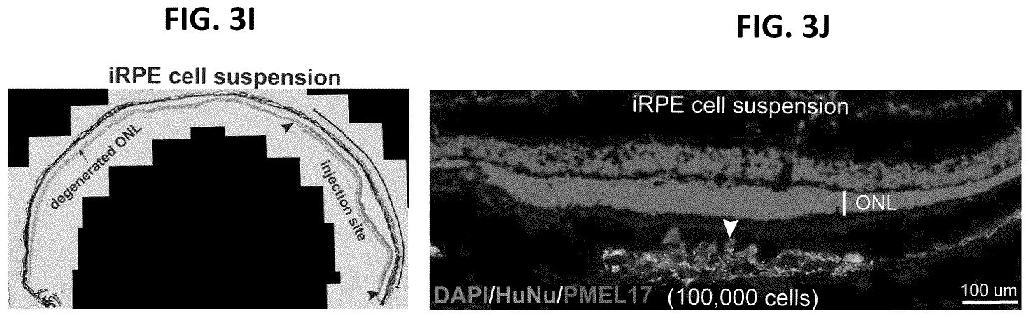

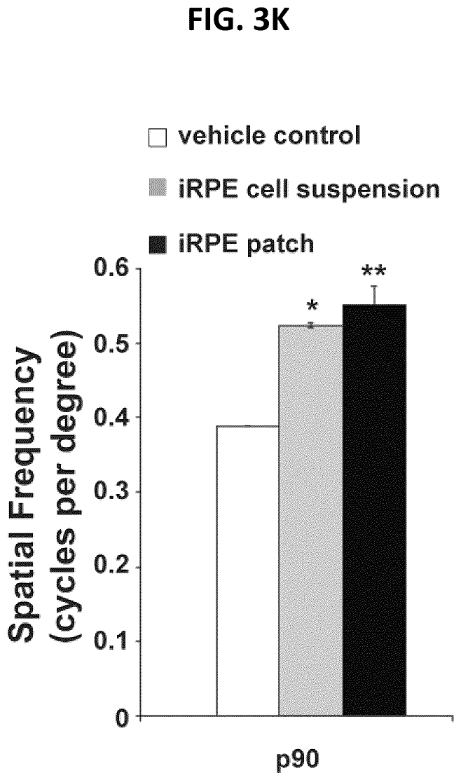

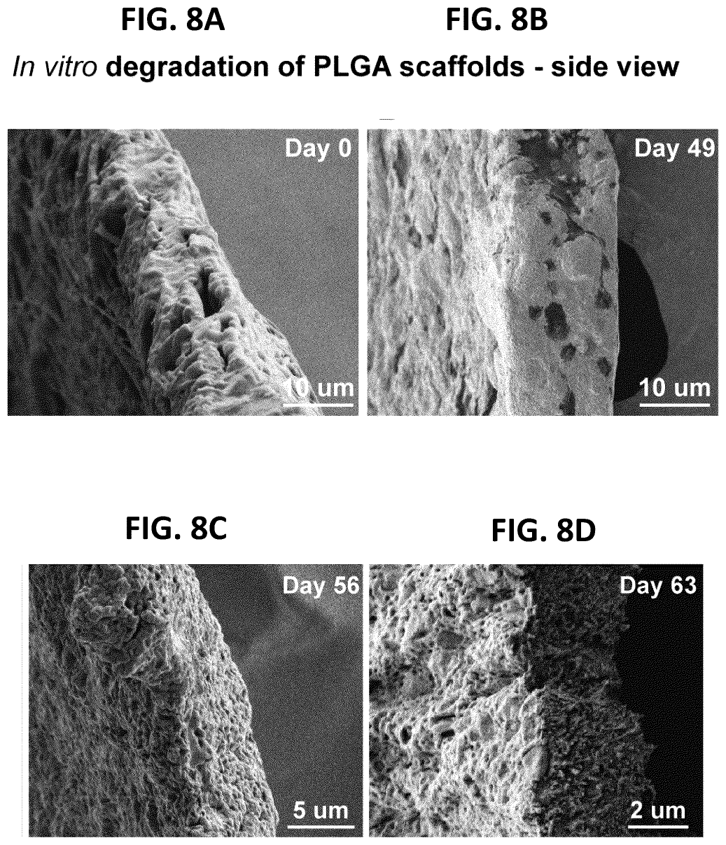

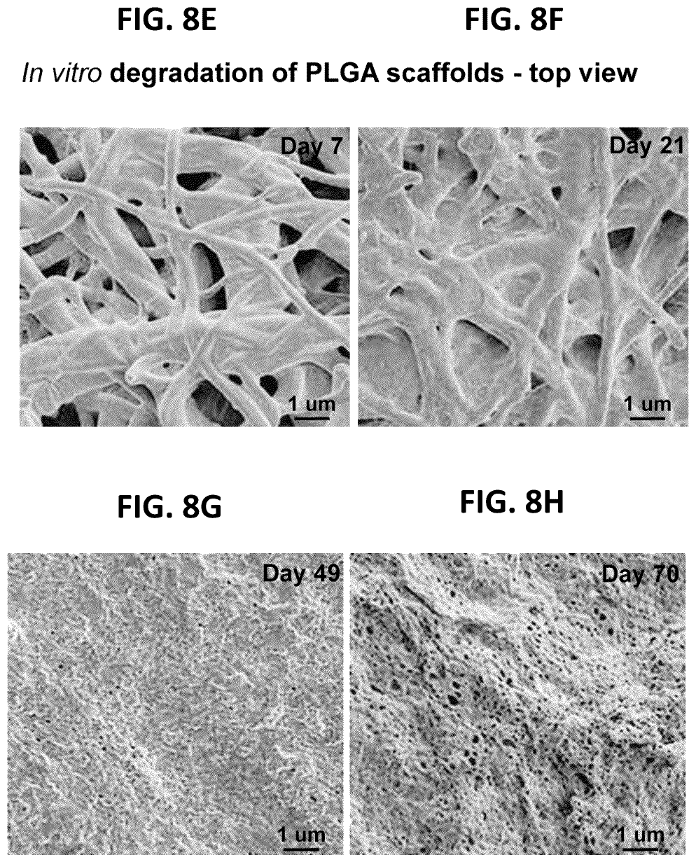

It was hypothesized that a biodegradable scaffold would provide suitable material for RPE cells to secrete extracellular matrix (ECM) to form a polarized monolayer. As the scaffold degrades, ECM and cells would constitute a native-like RPE tissue that would enhance the possibility of long-term integration of iRPE-patch in patients' eyes. Scaffolds used in the clinical-grade process were manufactured using poly-(lactic-co-glycolic acid)/PLGA (50:50 lactic acid/glycolic acid, IV midpoint 1.0 dl/g), with 350 nm mean fiber diameter previously shown to be optimal for RPE growth (Liu, et al., Biomaterials 35, 2837-2850 (2014); Stanzel et al., Stem Cell Reports 2, 64-77 (2014)). A single layer heat-fused nanofibers scaffold was selected for iRPE-patch manufacturing because of its high Young's Modulus that correlated with the ease of transplantation (FIGS. 2A, 2B). As expected for a biodegradable scaffold, it completely degraded in 80-90 days (SEM confirmed scaffold thickness at D49—10 μm; D56—5 μm; D63—2-4 μm; and D80-90—complete degradation; FIGS. 8A-8H; FIG. 3C). iRPE derived from all three patients matured and polarized on the scaffold as confirmed by high RPE65 and GPNMB expression and basal distribution of ECM proteins COLLAGEN IV and VIII (representative donor 3 clone C (D3C) shown in FIG. 2C). This suggested that iRPE cells synthesized a de novo Bruch's membrane equivalent, supporting our hypothesis that the 3D architecture of PLGA scaffold would trigger the secretion of ECM proteins by iRPE cells.

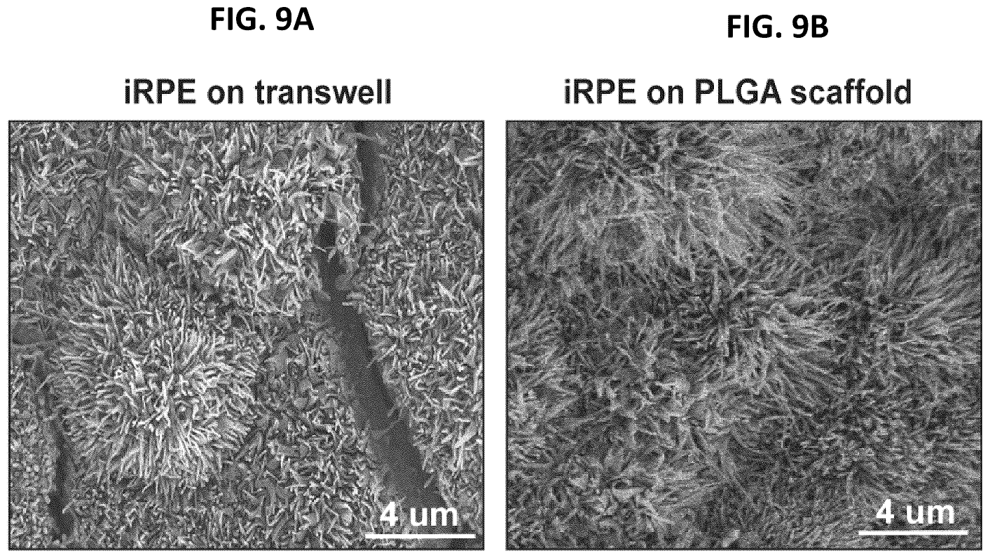

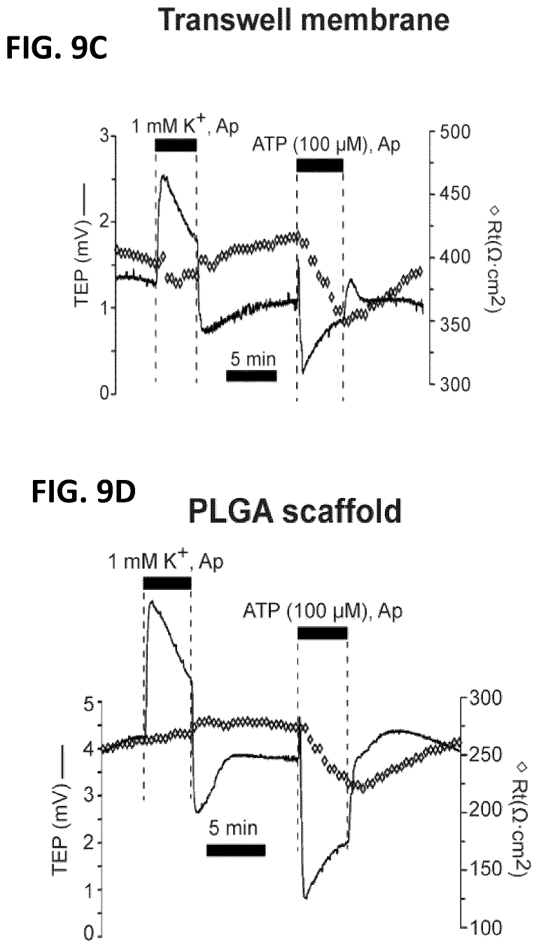

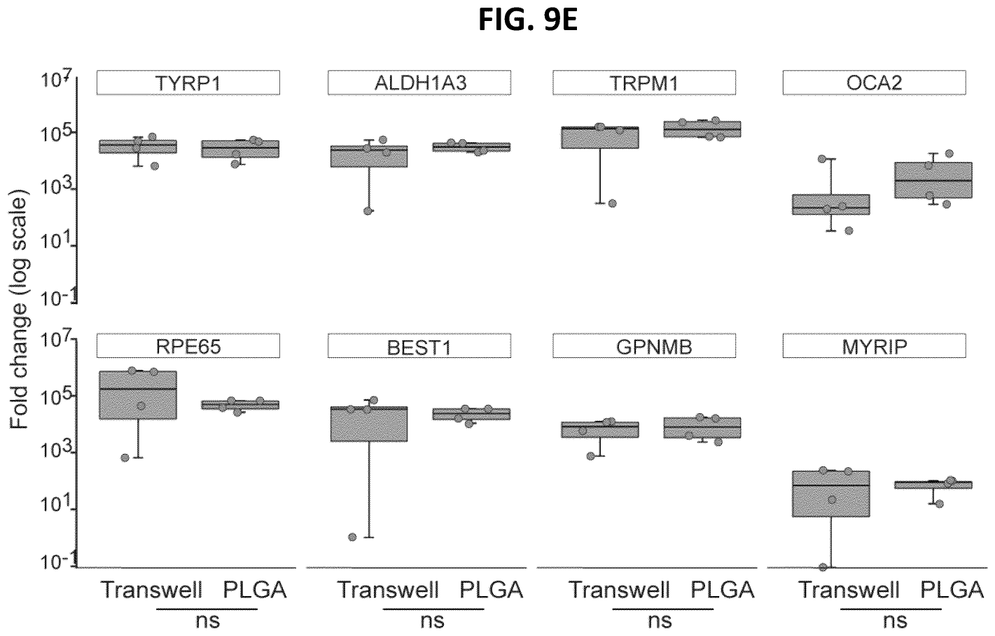

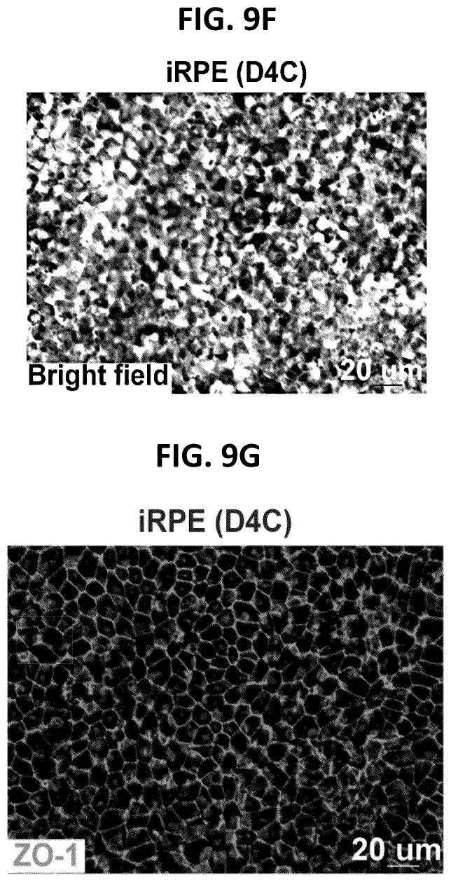

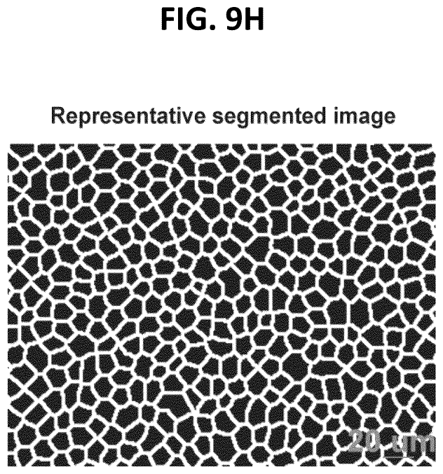

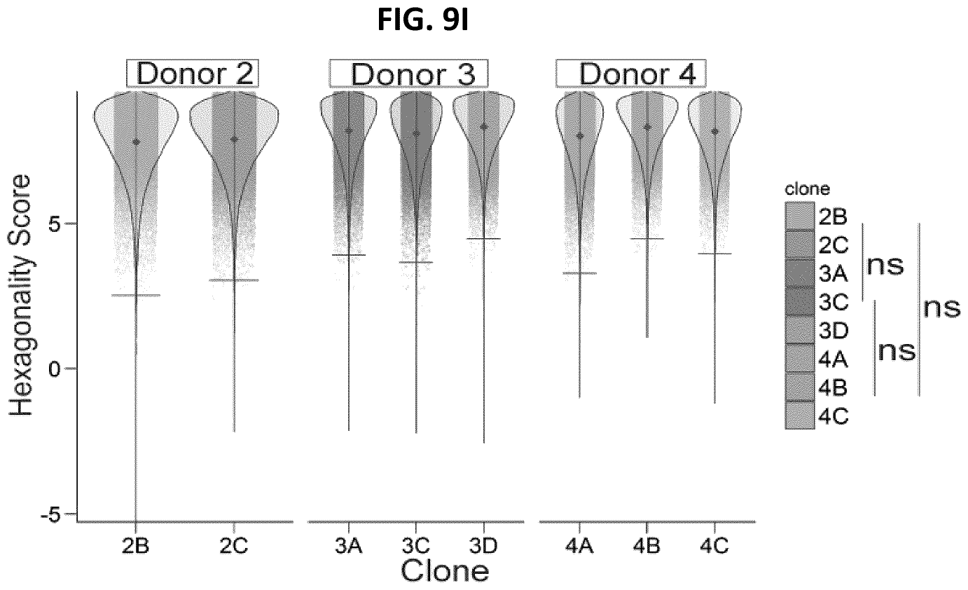

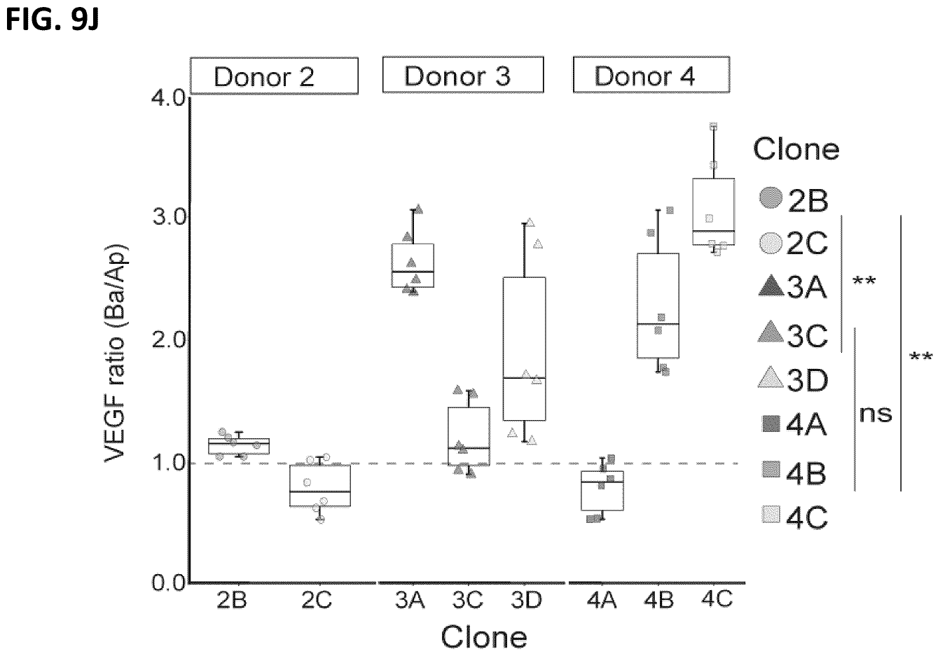

Previously, primary RPE and iRPE monolayer maturity and functionality was validated on semi-permeable transwell (polyester) membranes (May-Simera et al., Cell Reports 22, 189-205 (2018); Maminishkis et al., C Invest Ophthalmol Vis Sci 47, 3612-3624 (2006)). To determine if AMD-patient derived clinical-grade iRPE-patch on a PLGA scaffold and transwell behaved similarly, a structural, molecular, and functional comparison was made between D3C iRPE-patch on two surfaces. TEM and SEM confirmed the presence of dense apical processes with apically located melanosomes and tight junctions between neighboring cells; basal infoldings were only detected on PLGA scaffold (inset FIG. 2D; FIGS. 9A, 9B). Consistent with structural similarity between the two patch-types, both monolayers demonstrated similar electrical properties (FIGS. 8C, 8D) and similar expression of key RPE-specific genes OCA2, GPNMB, TYRP1, TRPM1, ALDH1A3, RPE65, and BEST1 with lower variability across samples on PLGA scaffolds as compared to the polyester membrane (FIG. 9E). Altogether, these results demonstrate that iRPE mature similarly on a polyester membrane or PLGA scaffold, but that there are native-like features on the PLGA scaffold, showing its superiority.

Donor-genetics is the biggest source of variation in cell types derived from iPSCs (Miyagishima et al., Stem Cells Transl Med 5, 1562-1574 (2016); Kajiwara et al., Proceedings of the National Academy of Sciences of the United States of America 109, 12538-12543 (2012)). To determine if such variation also exists in clinical-grade iRPE-patches derived from different patients and to develop criterion to functionally validate iRPE-patches before transplantation in patients, iRPE-patches were produced from all eight iPSC clones (FIG. 1C). A performance metric for “hexagonality” was designed to measure how close the iRPE-patches were to an ideal convex regular hexagonal pattern (See Example 1). Quantitative morphological assessment of iRPE-patches performed on images obtained with tight junction stain (ZO-1, FIGS. 9F, 9G) revealed similar hexagonality across all eight iRPE-patches (hexagonality score 8.1±0.1; out of 10; FIG. 9H), suggesting similar epithelial phenotype of iRPE patches from all three patients. Gene expression analysis of RPE-patches from all eight iPSC clones demonstrated similar expression of RPE-markers and suggested similar maturity of RPE monolayer across different patient iRPE (FIG. 2E). This conclusion was further corroborated by TER measurement during the last three weeks of iRPE-patch maturation from clones D3A, D3D, D4A. All three iRPE-patches demonstrated progressively increasing TER (250-1000 Ohms·cm²), suggesting gradual maturity of iRPE-patch (FIG. 2F). The iRPE-patch from six out of eight iPSC clones phagocytosed POS and secreted VEGF in a polarized fashion (FIG. 2G; FIG. 9I). Variation between different samples could be due to: (1) genetic differences between donor; (2) state of pluripotency of different clones; or (3) technical variation in different assays. To determine the main source of variation, a Principal Component Analysis (PCA) was performed for data obtained from all these assays (FIG. 2H). Interestingly, an iRPE-patch derived from D2B iPSC clone that differs in sequence from its donor also showed significant variation in functional output (FIGS. 1C, 2H). Thus, sequence changes likely account for variation in functional output from this sample. PCA performed by excluding D2B data revealed that the functional output of iRPE-patches clustered by donors (FIG. 2I), suggesting that patient genetics is likely a major source of variation in iRPE-patch function. Overall, this validation exercise confirmed the notion that a combination of sequencing, molecular, and functional readouts can be used to validate transplantable iRPE-patches.

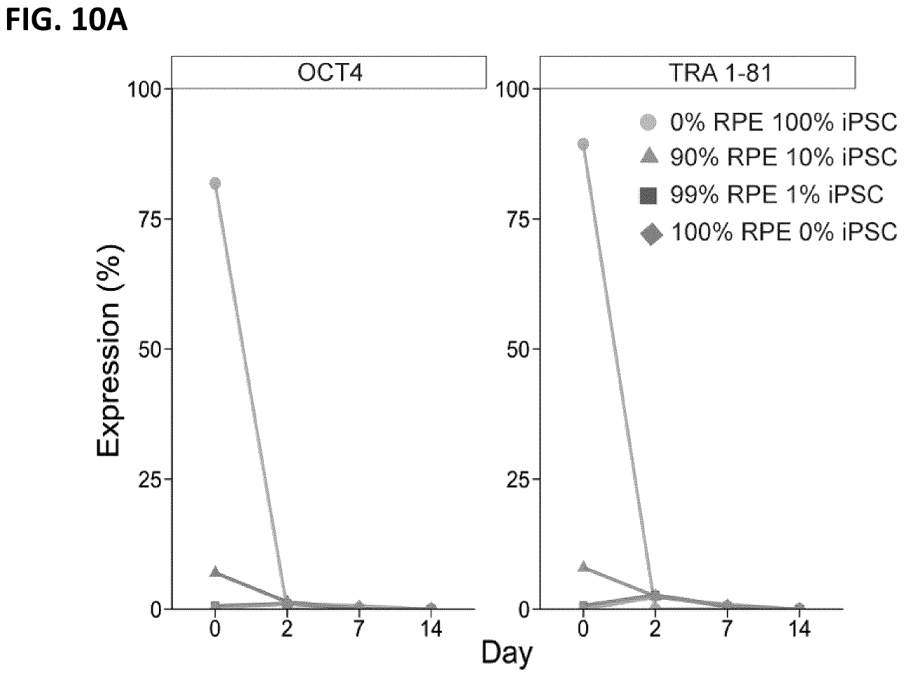

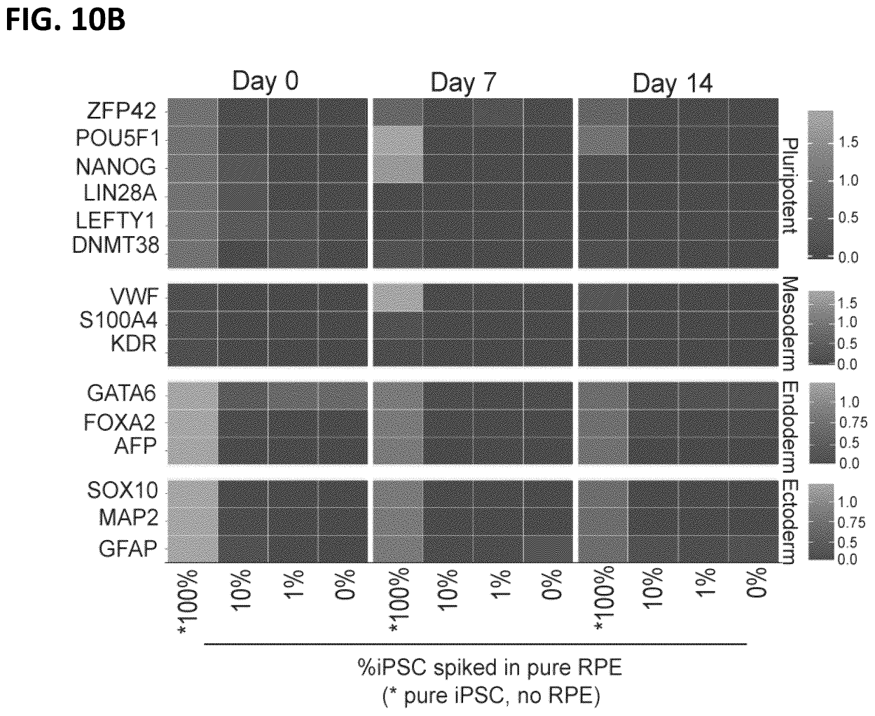

The purity of differentiated cells in the final product is a goal in developing a stem cell therapy. To check if iPSCs cannot survive culture conditions used for iRPE-patch maturation, an in vitro spiking study was performed. RPE cells mixed with 100%, 10%, 1%, or 0% iPSCs were seeded and cultured on PLGA scaffolds for 35 days. Flow cytometry confirmed that over 90% iPSCs had died within two days of culture and no iPSCs could be detected after D14 on PLGA scaffolds (FIG. 10A). Gene expression analysis also confirmed the absence of iPSCs, non-RPE cells, and the absence of non-RPE lineage markers in RPE cells in all cultures (except, as expected, for 100% iPSC; FIG. 10B). In conclusion, the in vitro spiking experiment confirmed that iPSCs do not survive RPE maturation conditions and the iRPE-patch contains less than (if any) detectable levels of iPSCs or non-RPE cells.

### Example 4

Clinical-Grade AMD iRPE-Patch Safely Integrates in the Eye and Shows Improved Efficacy Over Cell Suspension in a Rodent Pre-Clinical Study

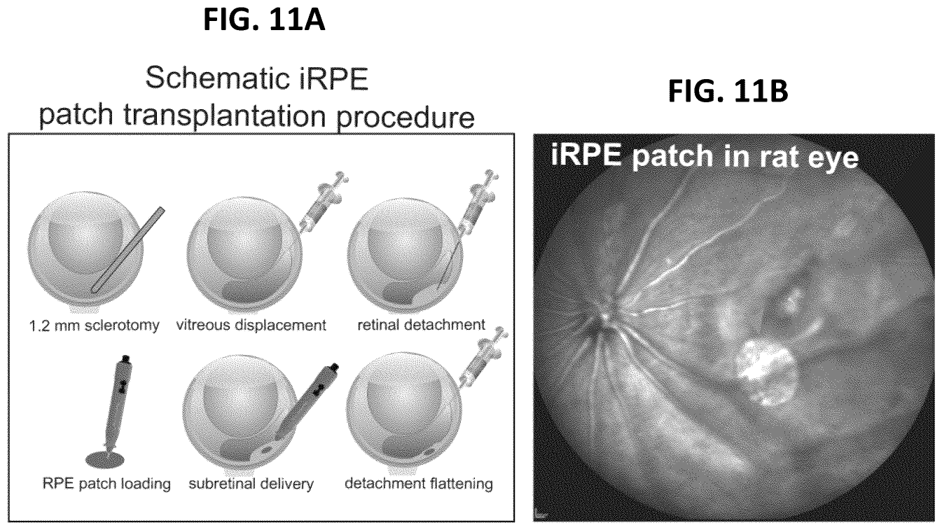

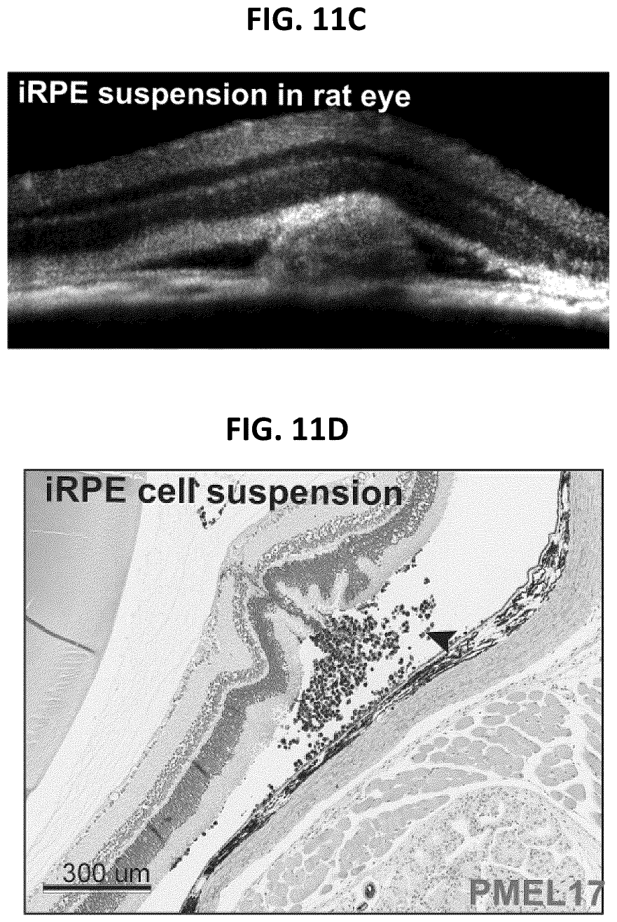

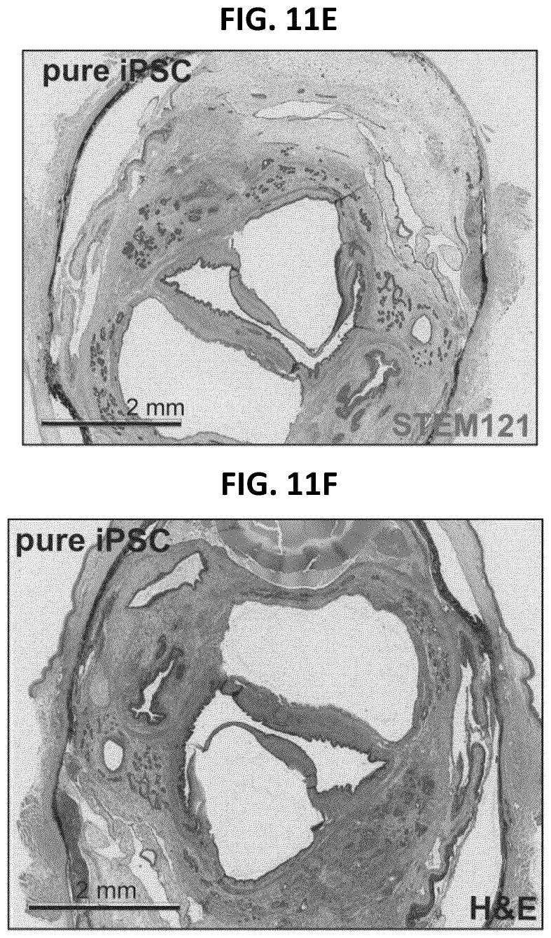

To test the long-term integration and the safety profile of AMD-iRPE-patch, 0.5 mm diameter (˜2,500 cells) clinical-grade patches were transplanted in the sub-retinal space of immunocompromised (Crl:NIH-Foxn1^{rmu}) rat eyes (FIGS. 11A, B). Fundus infrared imaging and optical coherence tomography (OCT) ten weeks post-surgery confirmed successful integration of the patch under the host retina (FIGS. 3A, B, horizontal line) and complete reattachment of the retina above the area of the transplant (FIG. 3B arrowhead). Histological analysis confirmed the OCT data that the AMD-iRPE-patch completely integrated on rat's Bruch's membrane (STEM121, human cells; FIG. 3C, arrowhead). In contrast, injected iRPE cell suspension rarely integrated into the rat RPE, consistent with previous observations that suspension cells do not form contiguous monolayer in the back of the eye (Diniz et al., Investigative Ophthalmology & Visual Science 54, 5087-5096 (2013)) (arrowheads FIG. 3D STEM121; FIG. 3F STEM121; FIG. 11D, PMEL17). AMD-iRPE cells in suspension or as a patch were negative for the Ki67 proliferation marker and no cases of tumor/teratoma were noted, whereas teratomas were observed when pure iPSCs were injected in the sub-retinal space (FIGS. 3E, 11E, F; Table 3). No signs of systemic toxicity of the transplant were noted in rats, all animals maintained their food consumption and body weight throughout the 10-week period (Table 3) suggesting the safety of human iRPE-cells as a transplantable patch. The data showing successful integration of iRPE-patches suggested that the patch would be more efficacious compared to cell suspension.

TABLE 3
Summary of preclinical rat and pig studies performed to demonstrate
safety and efficacy of clinical-grade AMD iPSC-RPE patch

| 1 | iPSC-RPE Patch Safety Study* in RNU-Crl:NIH-Foxn1^{rnu} Rats | | | |
|---|---|---|---|---|
| | (0.5 mm diameter iPSC-RPE patch) | | | |
| 1a | Sham surgery | 10 | 7 | 0/8 STEM121 |
| | | | | positive (2 unscheduled deaths) |
| 1b | iPSC-RPE sheet | 20 | 7 | 6/15 eyes showed integrated |
| | | | | iPSC-RPE patch |
| | | | | (one unscheduled death) |
| 2 | Cell Suspension Safety Study* in RNU-Crl:NIH-Foxn1^{rnu} | | | |
| | Rats (100,000 cells) | | | |
| 2a | Vehicle control | 5 | 5 | 0/5 STEM121 positive |
| 2b | Pure iPSCs | 10 | 5 | 3/10 teratoma |
| 2c | Scaffold pieces | 10 | 5 | 0/10 STEM121 positive |
| | cut from 8 mm² | | | |
| | PLGA punch | | | |
| 2d | iPSC-RPE cell suspension | 10 | 5 | 1/10 endo-ophthalmitis; |
| | | | | 9/10 STEM121 |
| | | | | and PMEL17 +ive; |
| | | | | 9/10 Ki67 −ive; no teratoma |
| 3 | Cell Suspension and Patch Efficacy Study in RCS | | | |
| | Rats (0.5 mm diameter iPSC-RPE patch or 100,000 cells) | | | |
| 3a | Sham surgery | 18 | 5/10 | PR degeneration |
| 3b | Suspension-100,000 cells | 11 | 5/10 | PR rescue in all eyes |
| 3c | Sheet-1 mm scaffold | 7 | 5/10 | 8/10 eyes showed integrated |
| | | | | iPSC-RPE patch and PR rescue |
| 4 | Empty PLGA Scaffold Safety Study in | | | |
| | WT Pig Eye (4 × 2 mm PLGA piece) | | | |
| 4a | Sham surgery | 3 | 10 | na |
| 4b | Empty PLGA scaffold | 3 | 10 | No inflammation, |
| | | | | slow degradation |
| | | | | of PLGA and |
| | | | | recovery of mfERG |
| | | | | signals |
| 5 | iPSC-RPE Patch Efficacy Study in Laser-injured Pig Eye | | | |
| 5a | Empty PLGA scaffold | 3 | 8 | Slow degeneration of PRs |
| | (4 × 2 mm) | | | |
| 5b | iPSC-RPE patch | 3 | 8 | Integration of iPSC-RPE patch |
| | (4 × 2 mm) | | | in pig eye and no |
| | | | | degeneration of PRs |
| 5c | iPSC-RPE | 3 | 8 | |
| | cell suspension | | | |
| | (100,000 cells) | | | |
| 5d | iPSC-RPE transwell | 3 | 8 | No degeneration of PRs, but no |
| | (4 × 2 mm) | | | integration of iPSC-RPE |
| | | | | transwell in pig eye |

*Body weight and food consumption was checked weekly for immunocompromised rats in the safety study. There was no apparent effect the patch or cell suspension on weight and food consumption of these animals.

To compare their efficacy, iRPE-patch and iRPE-cell suspension were transplanted in a previously established Royal College of Surgeon (RCS) rat model (35-38) between post-natal (p) day 21 and 28 with vehicle control (BSS+ or empty scaffold), a 0.5 mm diameter iRPE-patch (˜2,500 cells), or 100,000 iRPE cells in suspension. Both the iRPE-patch and cell suspension rescued overlying photoreceptors; notice the increased thickness of photoreceptor outer nuclear layer (ONL) in the transplanted area compared to the non-transplanted area (arrowheads mark human cells, human nuclear antigen/HuNu; human specific PMEL17; FIGS. 3G-J). The apparent difference in the thickness of ONL between iRPE-patch and iRPE-cell suspension transplanted rat retina is likely because of a relatively more invasive surgical procedure used to deliver a patch as compared to an injection of cells in suspension. Optokinetic (OKN) measurements (Douglas et al., Visual Neuroscience 22, 677-684 (2005)) confirmed that at p90 day the iRPE-patch and iRPE-cell suspension transplanted animals showed similar recovery as compared to vehicle control animals (FIG. 3K; Table 2). Overall, these rat experiments indicate that clinical-grade AMD iRPE-patch was able to completely integrate in the sub-retinal space of rodent eye as opposed to cell suspension that shows limited integration. The dose of cells on the patch is 1/40^{th} of the cells in suspension (2,500 cells on a 0.5 mm patch as compared to 100,000 iRPE-cell suspension). Despite that 40-fold difference, the iRPE-patch and cell suspension showed similar recovery by OKN. Thus, the AMD iRPE-patch was more efficacious compared to the cell suspension.

### Example 5

The Human Clinical Dose of AMD iRPE-Patch Integrates in the Eye of a Laser-induced RPE Injury Pig Model and Rescues Degenerating Retina

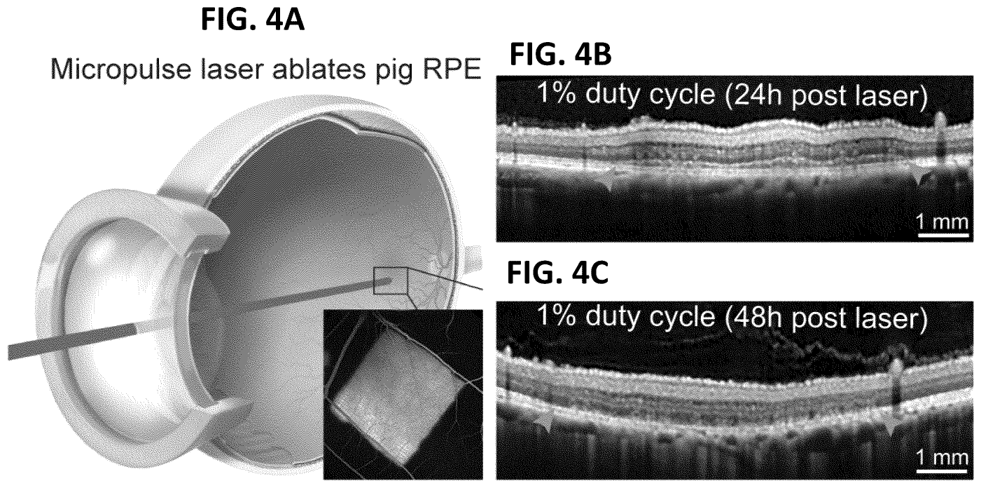

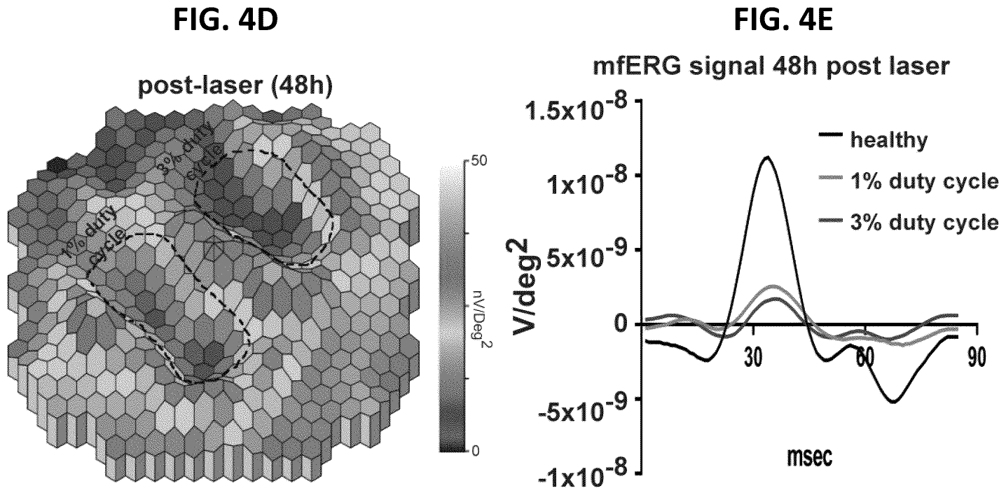

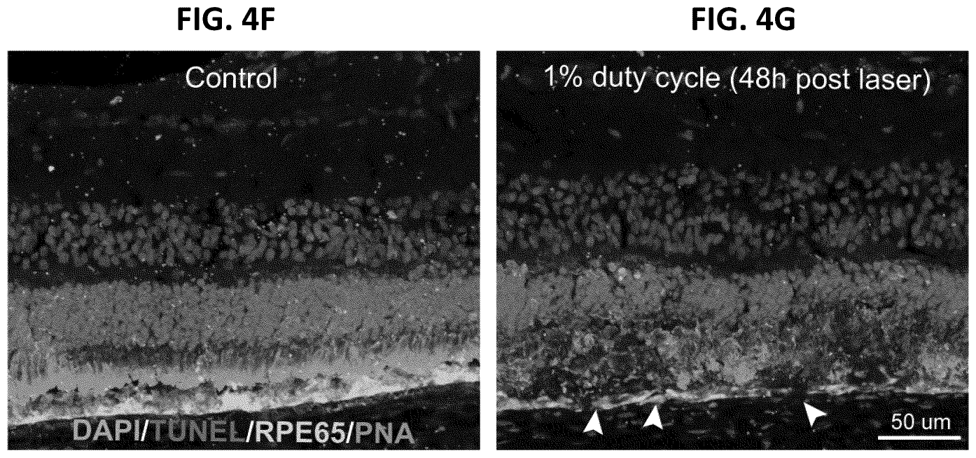

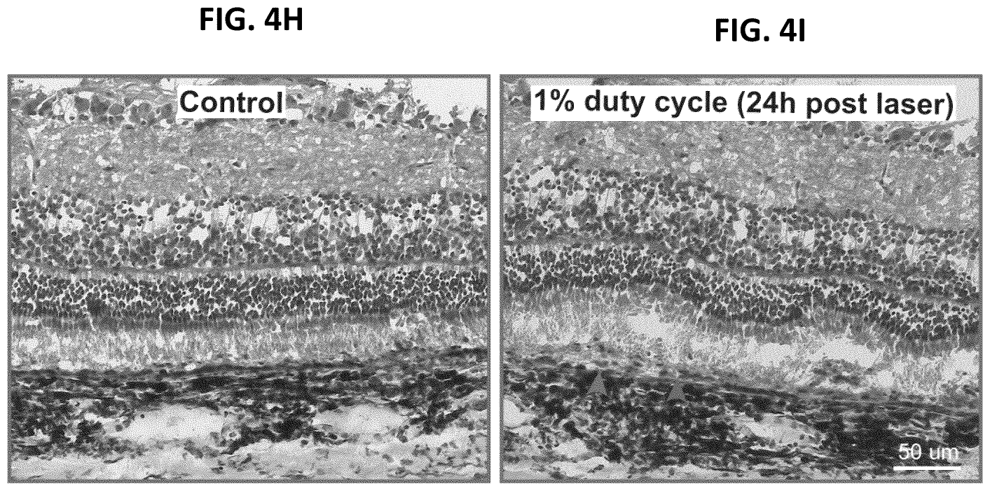

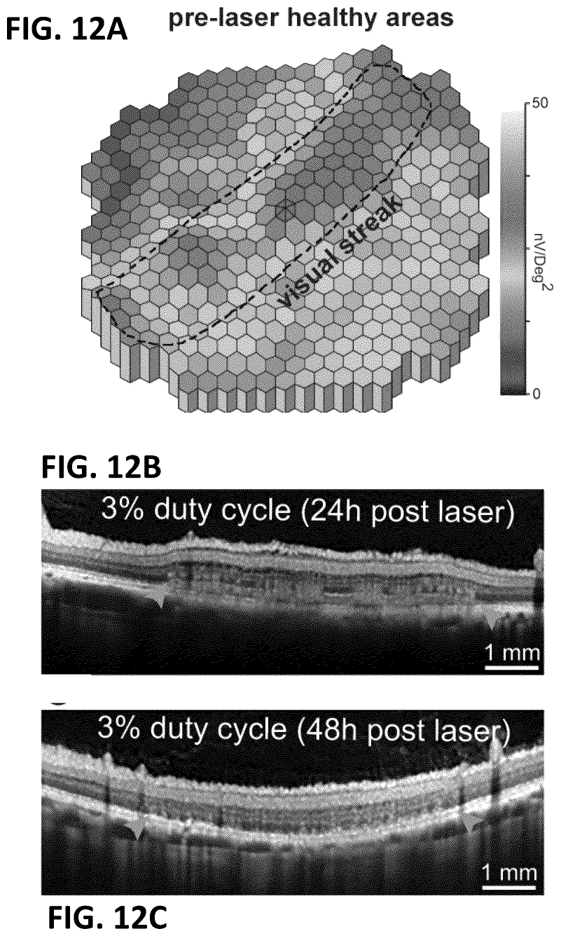

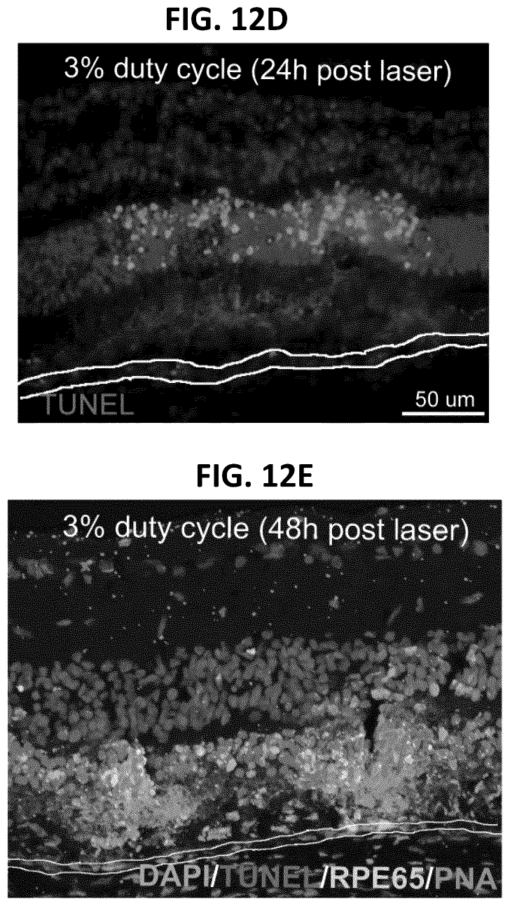

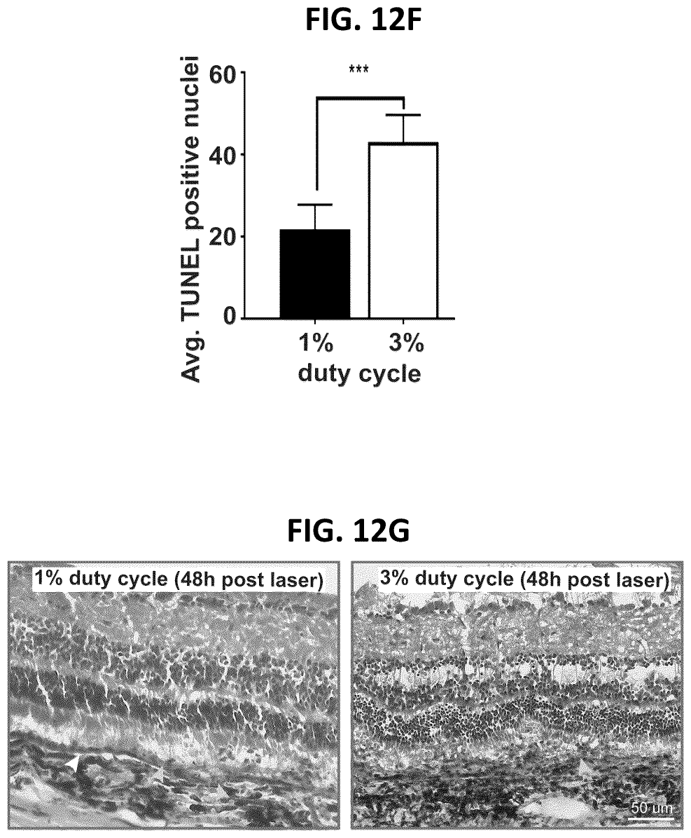

In RCS rat, the RPE monolayer is dysfunctional, but is still present, unlike what is seen in AMD patients. In order to test AMD iRPE-patch in an animal model with atrophied RPE, utilizing the entire human clinical-dose of 4×2 mm iRPE-patch, laser-induced RPE ablation was optimized in pigs. A property of melanin was exploited to efficiently absorb a 532 nm wavelength and a micropulse laser was used to selectively injure the pig RPE (Sivaprasad, et al., Surv Ophthalmol 55, 516-530 (2010)) (FIG. 4A). RPE injury was targeted at the pig visual streak, which contains the highest density of cone photoreceptors (FIG. 12A). OCT analysis of RPE/retina injury caused by 1% or 3% laser duty cycles (DC) at 330 msec exposure times revealed RPE detachment in 1% DC at 24h post-laser and RPE thinning at 48h (FIGS. 4B, 4C), whereas 3% DC additionally caused sub-retinal fluid accumulation at 24h post-laser and notable RPE/photoreceptor outer segment interface damage by 48h (FIGS. 12 B, 12C). Pre-laser mfERG confirmed similar electrical response across the visual streak in the retina, whereas post-laser both 1% and 3% DC laser treated areas showed comparable reduction in mfERG signals (FIGS. 4D, 4E, dotted line). OCT and mfERG results were confirmed at cellular levels by TUNEL staining combined with RPE65 and PNA immunostaining, which revealed apoptotic RPE and photoreceptor cells both in 1% and 3% DC laser-treated eyes, with higher apoptosis in photoreceptors at 3% as compared to 1% DC laser (FIGS. 4F, 4G, arrowheads FIGS. 12D, 12E). H&E staining further confirmed that both laser powers thermally damaged the RPE, but 1% DC caused less damage to photoreceptor outer segments both at 24 h and 48h (FIGS. 4H, 4I; 12F, 12G, arrowheads). Taken together, OCT, mfERG, and histology data suggest that 1% DC micropulse laser is preferred over 3% for inducing specific damage to pig RPE, while maintaining retinal electrical responses.

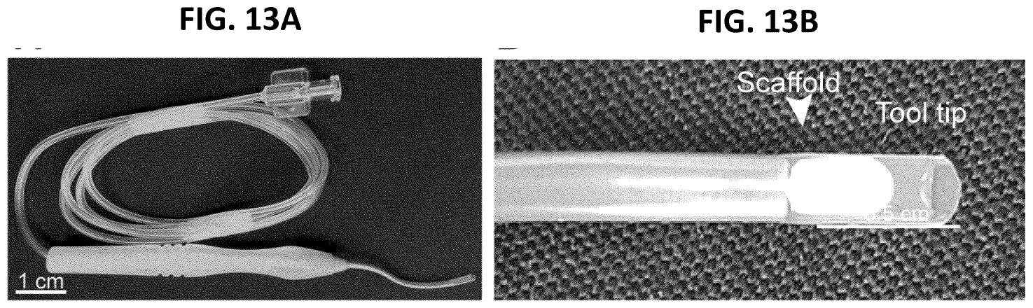

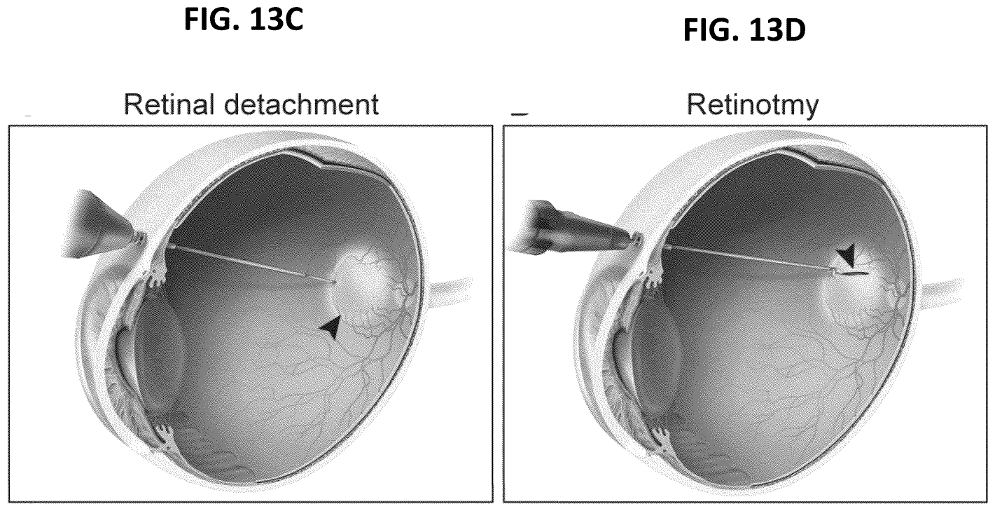

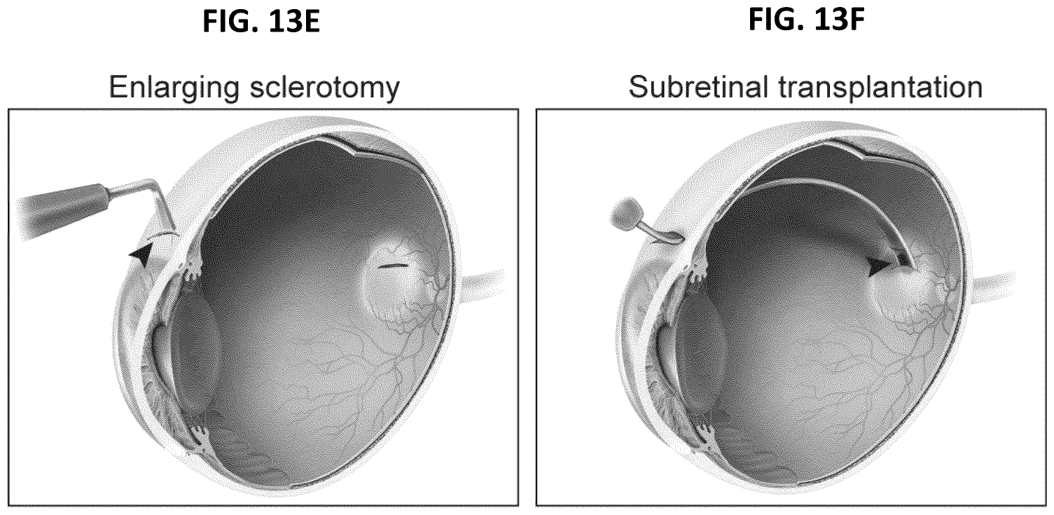

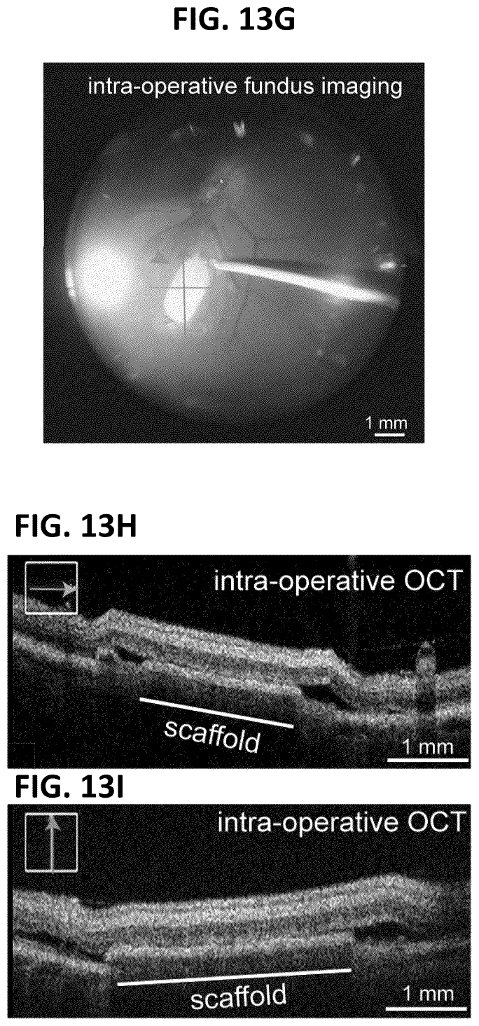

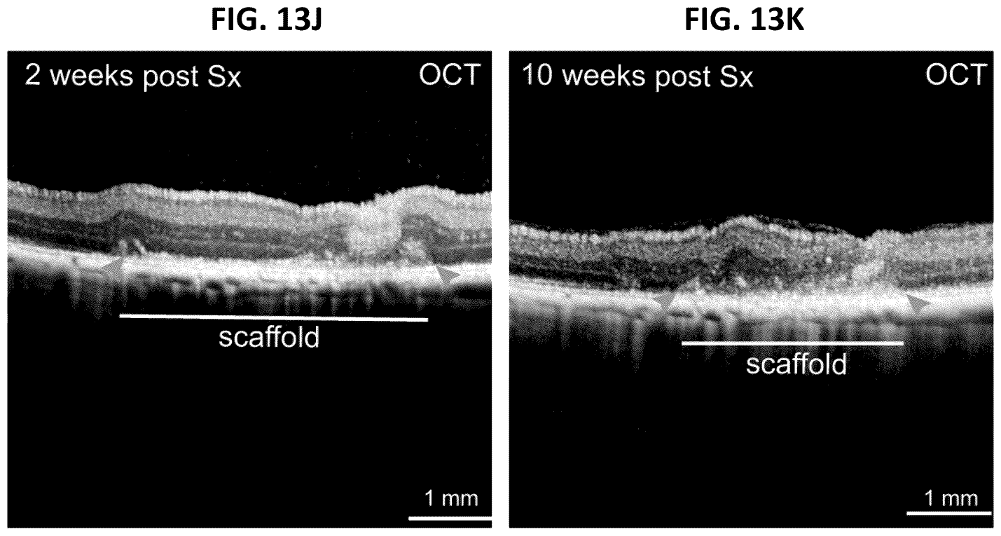

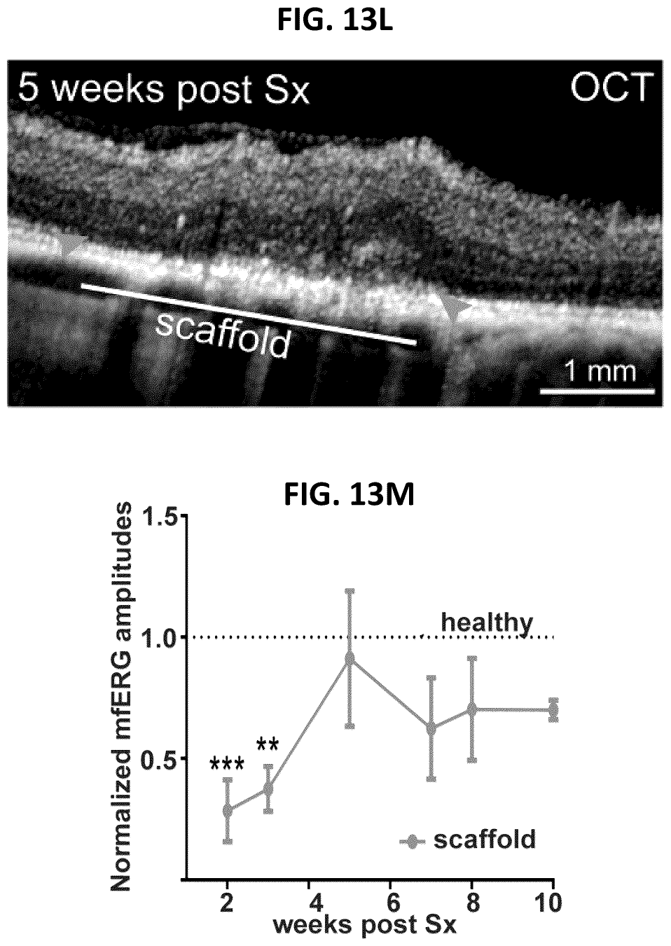

To deliver a 4×2 mm patch, a specific transplantation tool was designed with an S-shaped cannula that fits human (or pig) eye curvature and allows an easy delivery of the iRPE-patch while maintaining its orientation (FIGS. 13A, 13B, arrowhead). Surgery involved a four-port vitrectomy, posterior vitreous and retinal detachment, a 2.5 mm retinotomy, sclerotomy enlargement, and sub-retinal delivery of the iRPE-patch loaded in the tool (FIGS. 13C-13F, arrowhead). Intra-operative optical coherence tomography (iOCT) confirmed the correct sub-retinal delivery of the patch (FIGS. 13G-13I, arrowhead). Bare PLGA-scaffold without cells was first tested in the sub-retinal space of non-immunosuppressed, non-laser injured pigs to determine if PLGA degradation products (lactic acid and glycolic acid) caused any inflammation in the eye. OCT of pig eye two weeks post-surgery confirmed that the empty-scaffold can be delivered with minimal retinal damage (FIG. 13J, arrowhead). Ten weeks post-surgery there were no signs of inflammation, but the photoreceptor outer segment layer was thinner and showed retinal tubulations, suggesting damage of photoreceptors-likely because the empty-scaffold interfered with nutrient supply from the host RPE, the RPE-photoreceptor visual cycle, and the photoreceptor outer segment phagocytosis (FIG. 13K). Coinciding with scaffold degradation (5 weeks post-surgery), up to 80% of the N1P1 multi-focal ERG signal over the area of the implant had recovered, suggesting minimal damage caused by the empty-scaffold (FIG. 13M). These in vivo results are consistent with the lactic acid release profile of degrading PLGA scaffold. 83% of total lactic acid from the scaffold was released during in vitro culture. This included ˜70% of lactic acid that was released during the bulk degradation phase of our PLGA scaffold that started at week 3 and ended at the end of week 5 while scaffold was still in culture (Table 4). Furthermore, the highest amount of lactic acid released by the scaffold during the bulk degradation phase (0.0074±0.0014 mmol/L/scaffold/day) was 310× less than the systemic concentration of lactic acid (2.3 mmol/L) in blood (Wacharasint, et al., Shock 38, 4-10 (2012)) and 513× less than the lactic acid concentration in the eye at the RPE apical surface (Adler and Southwick, Ophthalmic Res 24, 243-252 (1992)). Overall, this data confirmed that the PLGA-scaffold is not inflammatory in the sub-retinal space of a pig eye and that the newly developed transplantation tool safely delivered the patch.

TABLE 4
Lactic released by the PLGA scaffold during degradation. Days 1-35 are the in vitro stage of the scaffold
and days 35 onwards are in vivo after transplantation. Grey highlights the days of PLGA scaffold bulk
degradation phase (days 19-36). Note, the bulk degradation of the scaffold occurs in vitro.

| | | | percent•lactic•acid |
|---|---|---|---|
| ¤ | Samples^{¤} | mmol/L/scaffold/day^{¤} | released/scaffold/week^{¤} |
| In-vitro^{.}stage^{.}of^{.}iPSC-RPE-patch^{.} | 1-1^{.}to^{.}1-6^{.}(days^{.}1-6)^{¤} | 0.00047 ± 0.00016^{¤} | 4.2% ± 1.7%^{¤} |
| maturation^{¤} | 2-1^{.}to^{.}2-6^{.}(days^{.}7-12)^{¤} | 0.00047 ± 0.00016^{¤} | 4.2% ± 1.7%^{¤} |
| | 3-1^{.}to^{.}3-6^{.}(days^{.}13-18)^{¤} | 0.00047 ± 0.00016^{¤} | 4.2% ± 1.7%^{¤} |
| | 4-1^{.}to^{.}4-6^{.}(days^{.}19-24)^{¤} | 0.00297 ± 0.00056^{¤} | 31.9% ± 4.0%^{¤} |
| | 5-1^{.}to^{.}5-6^{.}(days^{.}25-30)^{¤} | 0.00273 ± 0.00069^{¤} | 29.4% ± 7.5%^{¤} |
| | 6-1^{.}to^{.}6-6^{.}(days^{.}31-36)^{¤} | 0.00089 ± 0.00048^{¤} | 9.6% ± 4.9%^{¤} |
| In-vitro stage of iPSC-RPE-patch | 7-1^{.}to^{.}7-6^{.}(days^{.}37-42)^{¤} | 0.00047 ± 0 00016^{¤} | 4.2% ± 1.7%^{¤} |
| after^{.}transplantation^{.}in^{.}the^{.}sub- | 8-1^{.}to^{.}8-6^{.}(days^{.}43-48)^{¤} | 0.09047 ± 0.00016^{¤} | 4.2% ± 1.7%^{¤} |
| retinal^{.}space^{¤} | 9-1^{.}to^{.}9-6^{.}(days^{.}48-54)^{¤} | 0.00047 ± 0.00016^{¤} | 4.2% ± 1.7%^{¤} |
| | 10-1^{.}to^{.}10-6^{.}(days^{.}54-60)^{¤} | 0.00047 ± 0.00016^{¤} | 4.2% ± 1.7%^{¤} |

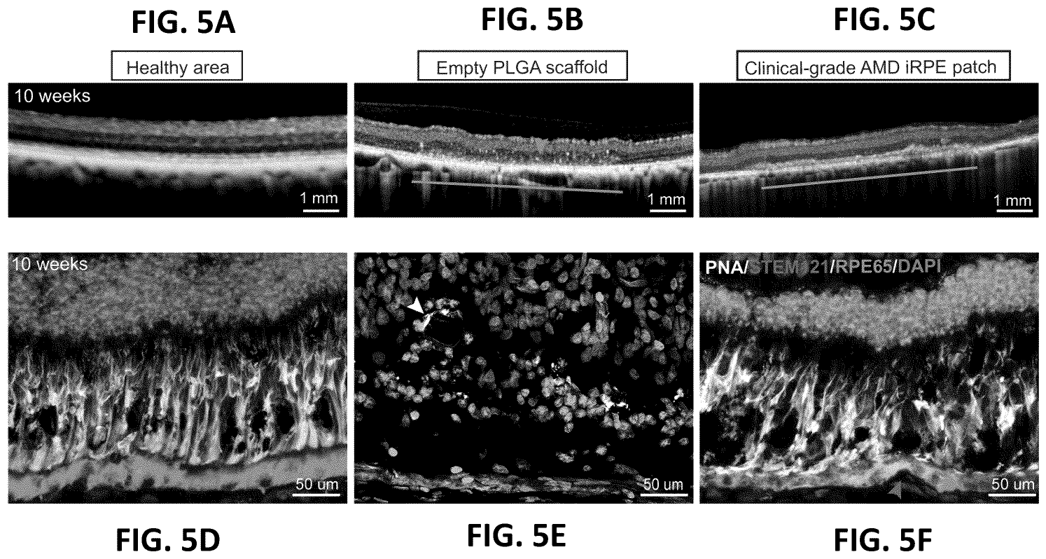

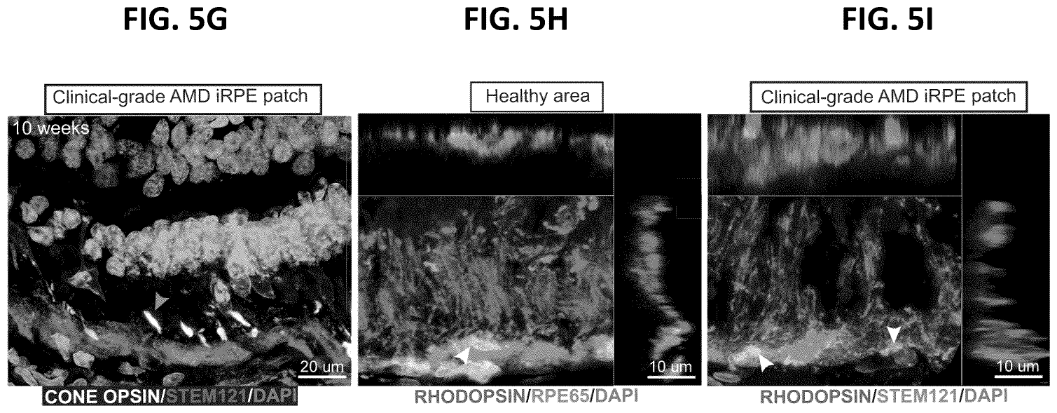

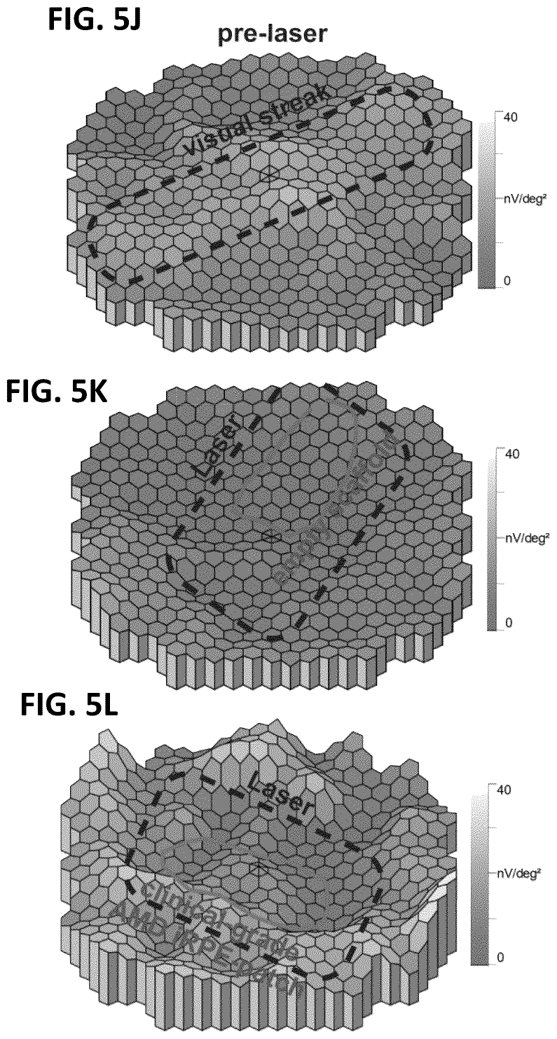

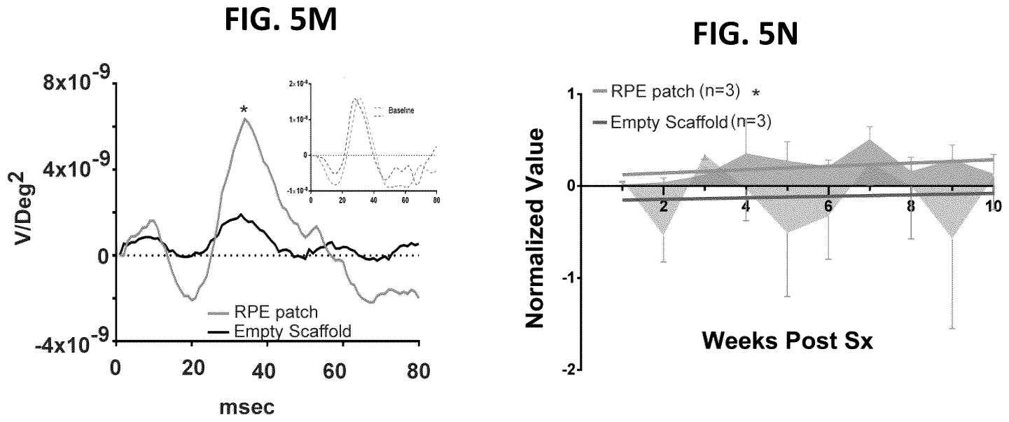

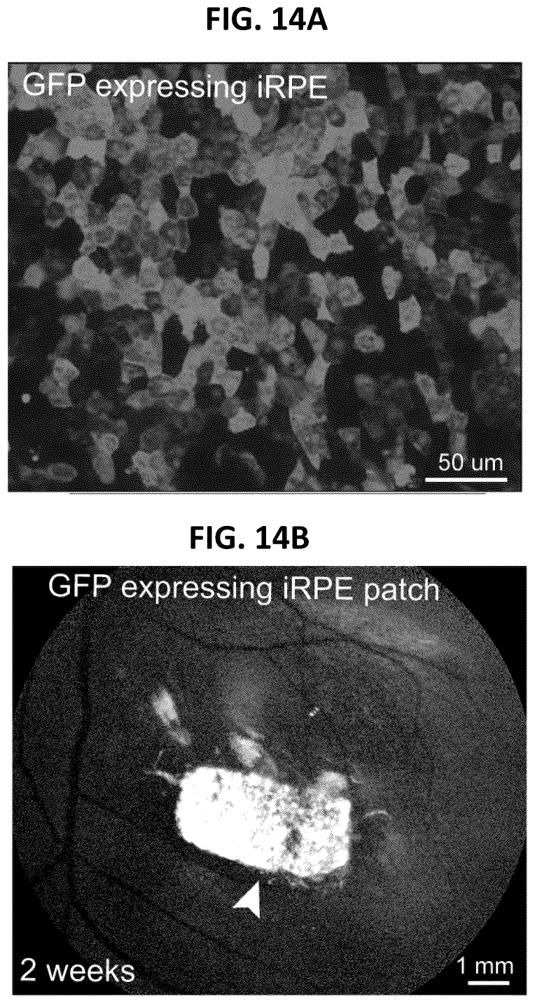

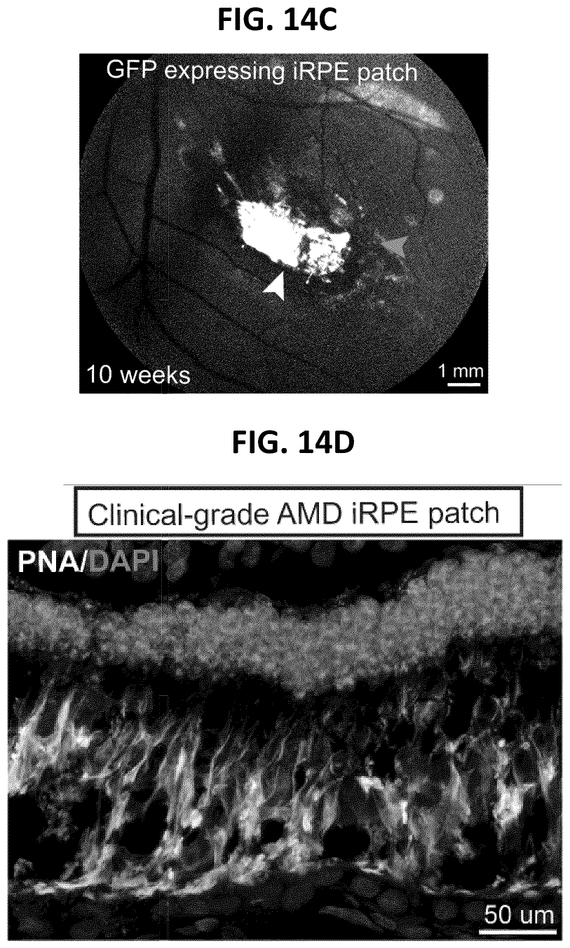

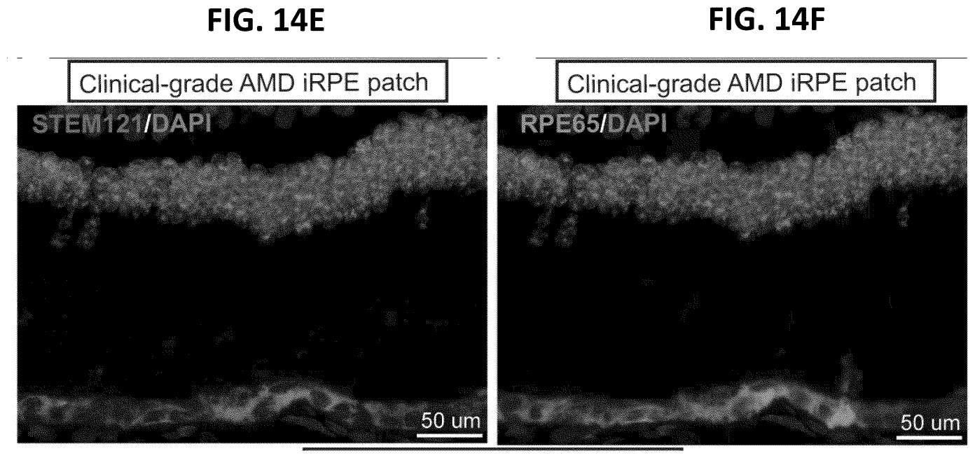

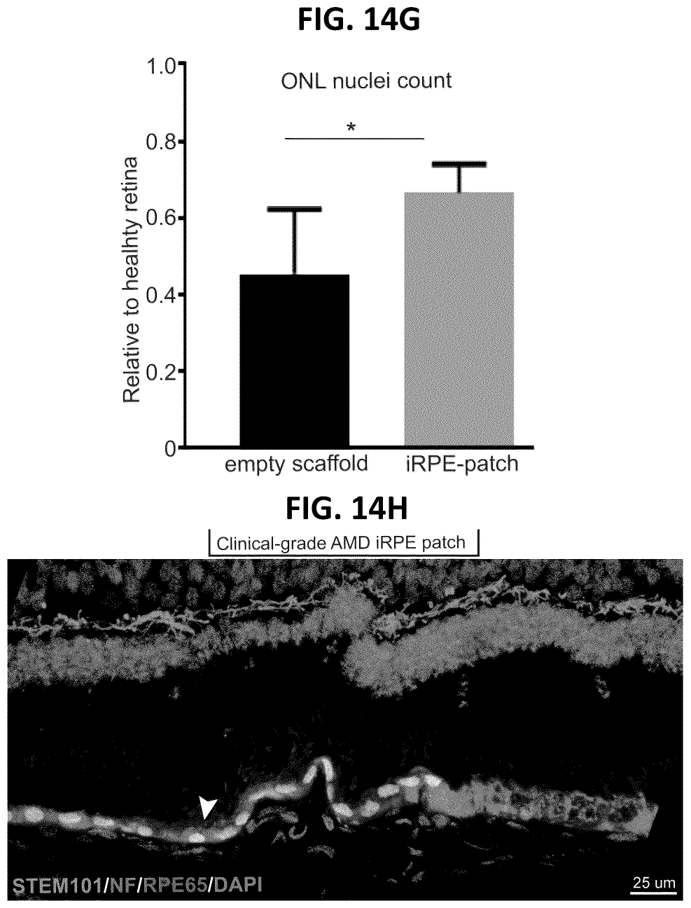

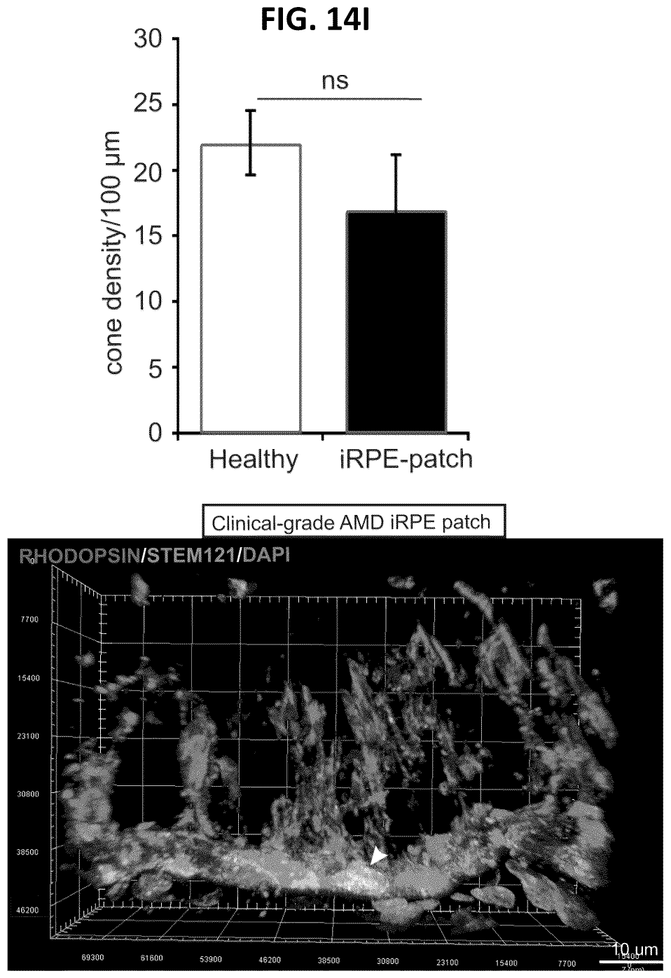

Fundus imaging of pig eyes transplanted with a GFP expressing iRPE-patch confirmed the survival of human cells for 10 weeks (FIGS. 14A-14C, arrowhead), achieved by suppressing the systemic and resident innate immune responses using prednisone, doxycycline and minocycline, and the adaptive immune response using tacrolimus and sirolimus (Santa-Cecilia et al., Neurotox Res 29, 447-459 (2016); Scholz et al., J Neuroinflammation 12, 209 (2015); Swijnenburg et al., Proc Natl Acad Sci USA 105, 12991-12996 (2008); Xian and Huang, Stem Cell Res Ther 6, 161 (2015)). The possibility was that, once the biodegradable PLGA scaffold degrades, the human clinical dose of AMD patient-derived iRPE-patch integrates on pig Bruch's membrane and is efficacious. A 4×2 mm patch was transplanted in a pig eye over an area with laser-induced RPE ablation. OCT confirmed that as the PLGA-scaffold degraded over 10 weeks, the clinical-grade iRPE-patch integrated in pig eye and the retina above the iRPE-patch maintained both inner and outer retinal layers as compared to the animals transplanted with an empty PLGA scaffold, where retinal tubulations were evident (FIGS. 5A-C; arrowhead in 5B and 5E). Immunostaining confirmed integration of the AMD-iRPE-patch in laser-injured pig eye and a mature phenotype of transplanted cells as validated by strong RPE65 immunostaining in iRPE-cells (STEM121; RPE65, FIGS. 5D-F, FIGS. 14D-14F). PNA staining confirmed improved organization of photoreceptor outer segments over iRPE-patch transplanted retina as compared to empty scaffold transplanted retina (White PNA, FIGS. 5D-F).

To quantify differences in photoreceptor rescue between empty scaffold and iRPE-patch transplants, the number of photoreceptor nuclei in the ONL above the area of both transplants was counted, and results were comparted to the adjacent healthy retina. The analysis demonstrated that in the ONL over the empty scaffold, the number of nuclei were 42% of the adjacent healthy area, whereas over the iRPE-patch area the number of nuclei were 73% of the heathy area (p<0.05, T-Test; FIG. 14G). To rule out the possibility that STEM121 labeling was not caused by pig RPE phagocytosing human iRPE cells, immunostaining was performed for a nucleus-specific human antigen (STEM101) (FIG. 14H). This data showed specific nuclear labeling only in a part of RPE providing additional evidence of integration of human iRPE-patch in the pig eye.

Because laser-damage and subsequent iRPE-patch transplantation was performed in the visual streak area of pig eye, it was determined if the human RPE cells were able to preserve pig cone photoreceptors. Immunostaining for specific cone opsins (S, L, and M) confirmed the preservation of pig cone photoreceptors above the area of the iRPE-patch (FIG. 5G, arrowhead). To test functional integration of human iRPE-patch in the pig eye, it was tested if human RPE cells are able to phagocytose pig photoreceptor outer segments (POS). Rhodopsin staining of healthy pig retina and retina transplanted with clinical-grade human iRPE-patch revealed phagocytosed POS inside human RPE cells, similar to what is seen for the native pig RPE (arrowheads in FIGS. 5H, I). Preservation of cone photoreceptors and functional integration of human RPE inside the pig eye prompted testing of recovery of electrical responses from laser-damaged pig retina over the area of iRPE-patch. Heatmaps of mfERG responses showed improved signal over the iRPE-patch transplanted laser-damaged visual streak area as compared to the empty scaffold transplanted pigs (FIGS. 5J-L). To address issues regarding regional variability in the placement and extent of laser injury on the effect of the iRPE-patch, a linear mixed effect (LME) analysis of all of the mfERG components was used. LME analysis of all mfERG components (N1, N1 width, P1, P1 width, NIP1, Area Under the Curve (AUC), and Scalar Product) revealed a significant difference between the iRPE-patch and the empty patch over 10 weeks (p<0.05) (FIG. 5M). This observation was further confirmed in the linear regression analysis of Area Under the Curve that also showed a significant difference between the two groups (FIG. 5N, y intercept difference, p<0.05). In summary, these results confirmed that clinical-grade AMD-iRPE-patch integrated with the pig retina and rescued pig photoreceptors after laser-injury of pig RPE, and that iRPE-patches from different patients demonstrate similar efficacy responses.

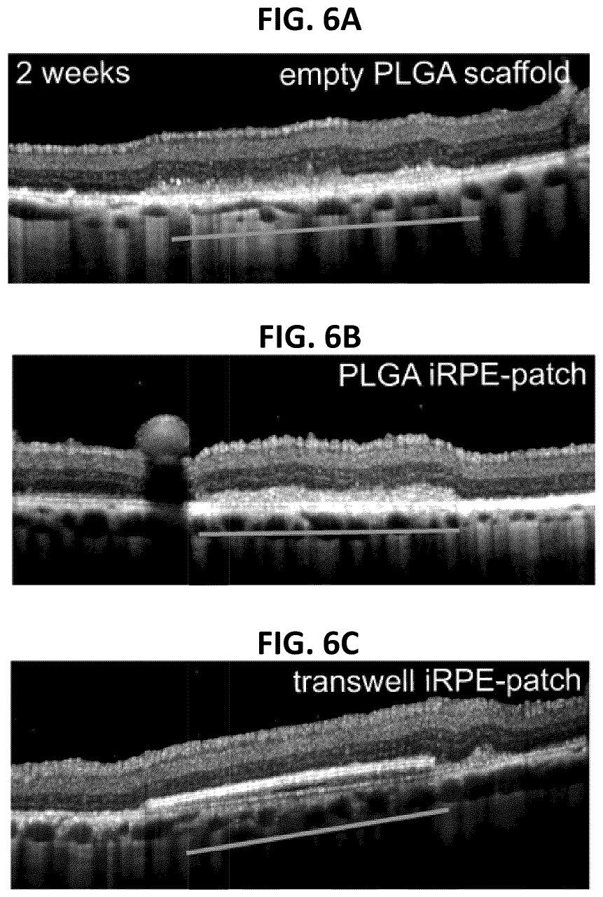

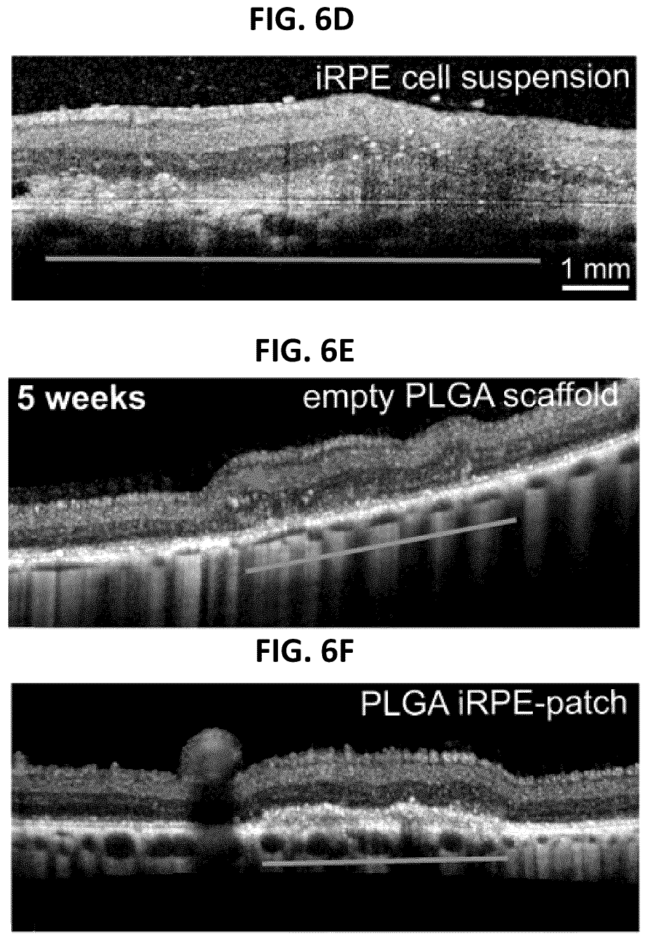

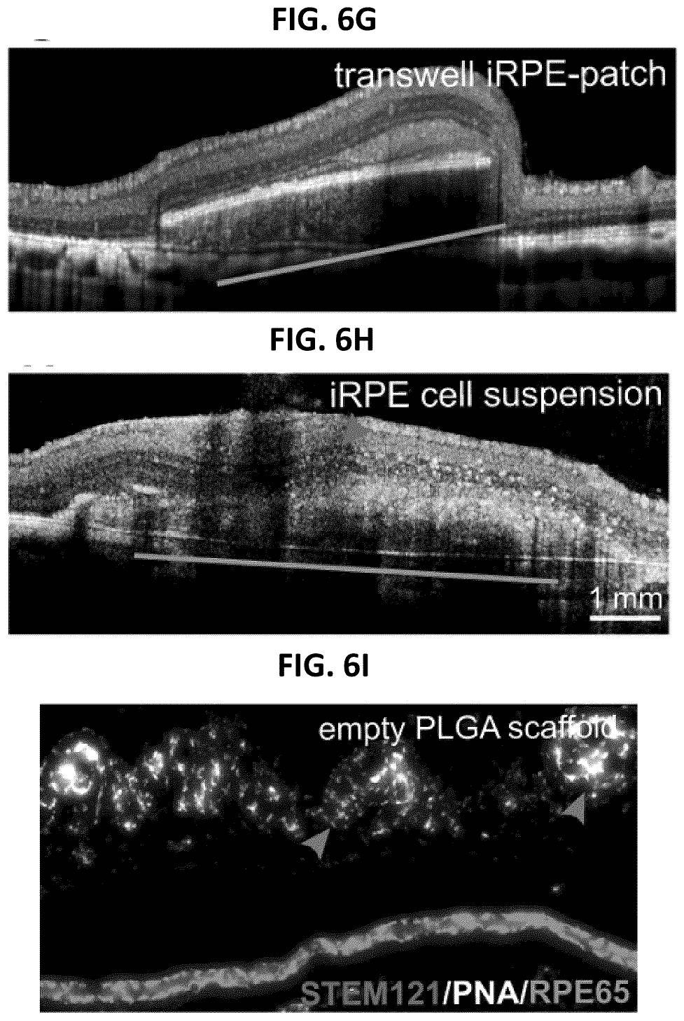

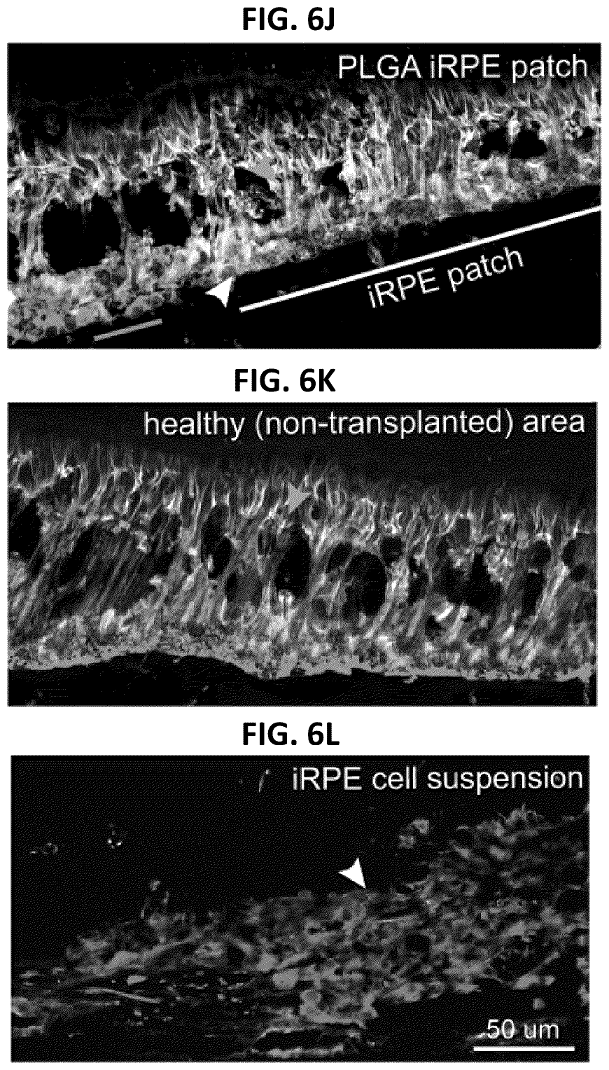

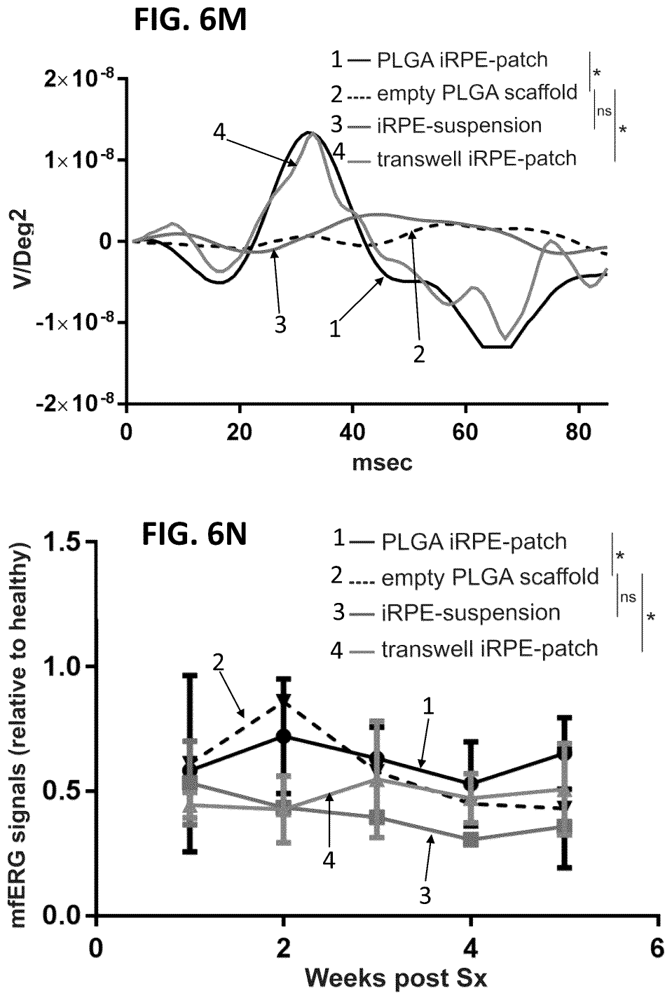

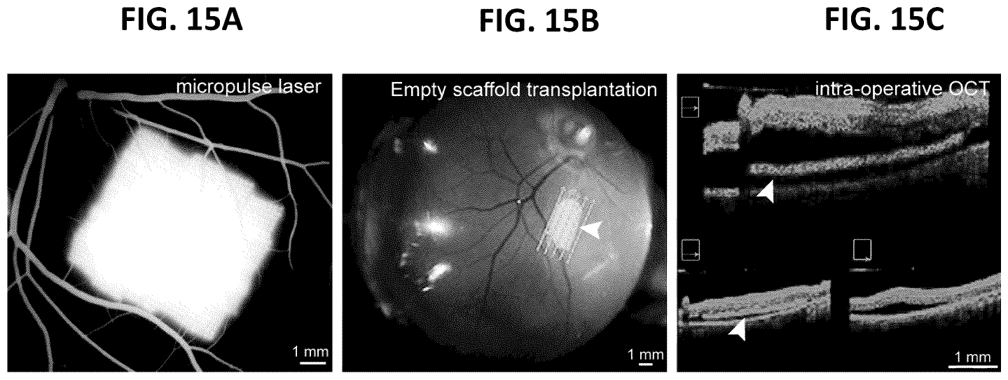

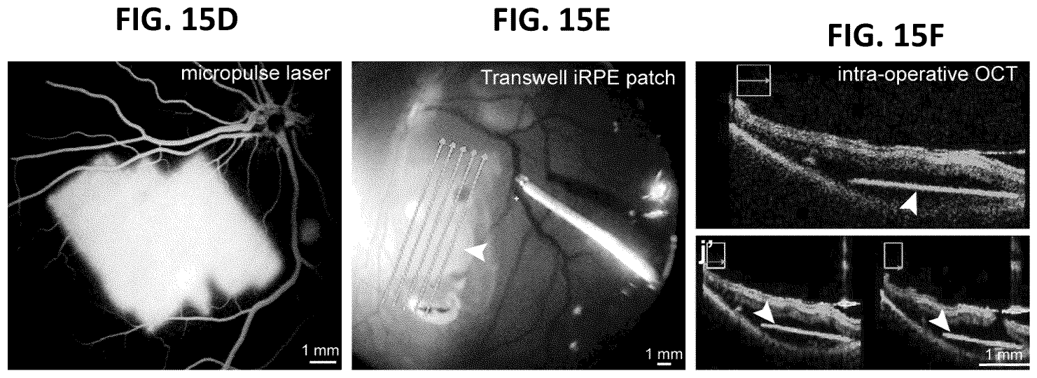

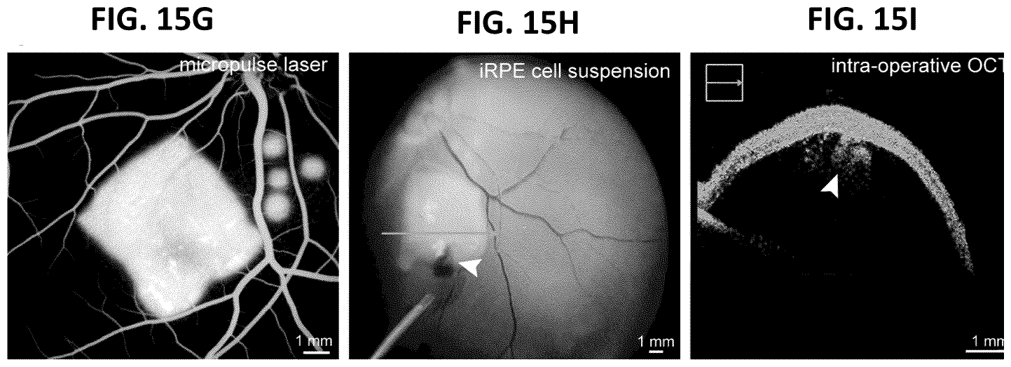

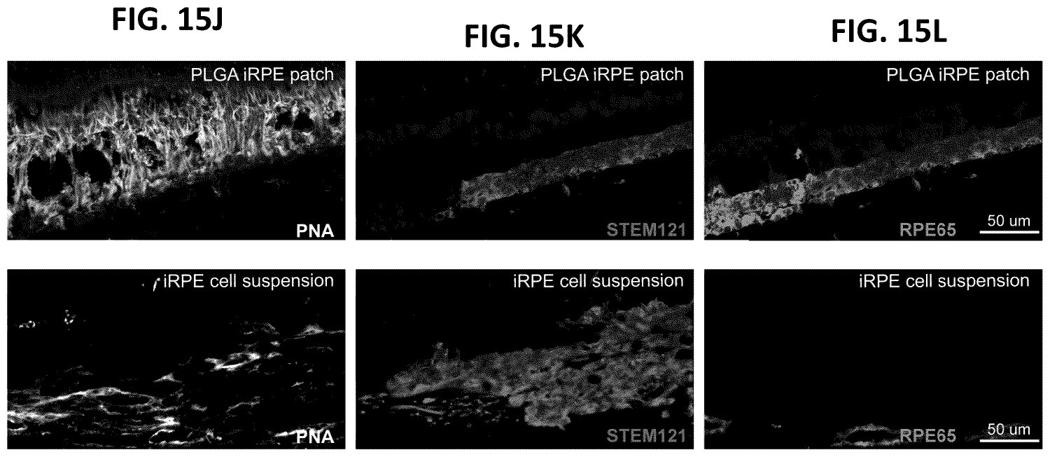

To perform a comparative analysis of PLGA-iRPE-patch with transwell-iRPE-patch (non-degradable transwell membrane) and iRPE cell suspension with empty scaffolds, all four transplants were tested in pigs with laser-ablated RPE (FIGS. 15A-I). Two weeks post-surgery, OCT confirmed correct delivery of all four transplants with minimal signs of inflammation (FIGS. 6A-D). At 5 weeks post-surgery, empty scaffold and iRPE suspension showed disruptions in the outer nuclear layer and external limiting membrane of the retina and structures that were reminiscent of outer retinal tubulations often seen in degenerating retina (47) (arrowhead in FIGS. 6E, H). In contrast, OCT showed that retina overlying iRPE-patches (both PLGA and transwell) did not exhibit any such retinal tubulations and both the outer nuclear layer and external limiting membrane appeared intact (FIGS. 6F, G). The OCT analysis was consistent across all three pigs evaluated for each treatment. Immunohistochemistry of the PLGA-iRPE-patch confirmed integration and post-transplantation maintenance of mature phenotype of human RPE cells in the back of the pig eye (compare 6I-K; White, STEM121, human-specific; grey, RPE65), and maintenance of photoreceptors above human iRPE-patch but not above the empty scaffold or cell suspension transplants. (PNA, photoreceptors; FIGS. 6I-L; FIGS. 8SD-I; Table 2). In contrast, ONL above the empty scaffold and iRPE suspension shows tubulations and degeneration as seen in OCT (arrowheads in FIG. 6I). Furthermore, unlike the iRPE-patch, iRPE cells in suspension lose the expression of RPE maturity maker RPE65 (arrowhead in FIG. 6L). Without being bound by theory, this is likely because trypsinized RPE cells, if not re-organized as a confluent monolayer, cannot maintain a stable epithelial phenotype (Radeke et al., Genome Med 7, 58 (2015)). Consistent with the OCT and immunostaining data, mfERG confirmed higher recovery of mfERG individual waveforms and integrated data over 5 weeks in the lasered area both in PLGA-iRPE-patch and transwell-iRPE-patch, as compared to empty PLGA-scaffold or iRPE cell suspension (FIGS. 6M, N). These results demonstrate that monolayer iRPE-patch is superior than cell suspension in rescuing retinal degeneration in laser-injured pig eye.

A successful autologous cell therapy requires an efficient and reproducible manufacturing process that generates a safe and efficacious product. The disclosed clinical-grade process provides reproducibility of the manufacturing process, and ensures safety and efficacy of the clinical product (Schwartz et al., Lancet 385, 509-516 (2015); Mandai et al., N Engl J Med 376, 1038-1046 (2017); Kamao et al., Stem Cell Reports 2, 205-218 (2014)). The manufacturing process was developed using CD34+ cells of three advanced-stage AMD (geographic atrophy) patients (Mack, et al., PLoS One 6, e27956 (2011); Badenes et al., PLoS One 11, e0155296 (2016); Badenes et al., PLoS One 11, e0151264 (2016)). Passage10 working banks of clinical-grade iPSCs were generated up to three banks per patient were validated for iPSC critical quality attributes (Table 2).

In addition to their pluripotency and correct G-band karyotyping, there were two safety attributes, specifically the loss of reprograming plasmid and oncogene sequencing. All nine clones from three patients lost their reprograming plasmid by passage 10. Without being bound by theory, this may be because a low-copy number EBNA-origin of replication-based plasmid (Carr et al., PLoS One 4, e8152 (2009)) was used. None of the iPSC clones acquired any oncogenic mutations during reprograming or expansion, and eight out of the nine iPSC clones had no observed sequence changes (Kwon et al., Proc Natl Acad Sci USA 114, 1964-1969 (2017)) (FIG. 1C).

One iPSC clone, D2B, which showed several sequence and copy number alterations also showed variation in iRPE-patch functional output (FIG. 2H). Without being bound by theory, these sequence changes may be the reason for variation in functional output at the iRPE-patch level. A combination of iPSC oncogene sequencing and test article functional analysis can be performed to identify transplantable derivatives of iPSCs.

Clinical-grade differentiation of iPSCs into a mature and polarized RPE-patch on a biodegradable PLGA-based scaffold (FIGS. 1, 2) was demonstrated. iPSC differentiation into a transplantable RPE-patch takes ˜10 weeks (Kamao et al., Stem Cell Reports 2, 205-218 (2014)). The process is user-independent (FIGS. 7M-7O) and scalable to multiple iPSC clones (FIGS. 1, 2). PCA suggested that patient genetics is likely the largest single contributing factor to any variability. Although iRPE-patches derived from different patients show different functional responses, the patches could be functionally validated to produce a transplantable product.

As compared to ESC-RPE suspensions (Schwartz et al., Lancet 379, 713-720 (2012); Schwartz et al., Lancet 385, 509-516 (2015)), iRPE-patches contain a fully-polarized monolayer of cells that integrate into the Bruch's membrane of immunocompromised rats, and in pigs after laser-induced RPE injury (FIGS. 3C, 3H, 3J, 5D-5I, 6I-6L). This outcome probably reflects a coordination of continuous PLGA scaffold degradation and ECM production by iRPE, which facilitates integration with the host Bruch's membrane. The results show that iRPE cells on PLGA scaffolds make Bruch's membrane proteins Collagen IV and Collagen VIII (FIG. 2C). Furthermore, unlike cell suspension that may not be able to perform most of RPE functions, the polarized RPE monolayer on PLGA-iRPE-patch and transwell-iRPE-patch performed a multitude of RPE functions (FIG. 2, FIG. 9). In combination, these properties of iRPE-patch suggest a mode of action for improved efficacy seen with the patch approach. RPE ablation in the laser-injury pig model was similar to the loss of RPE in advanced AMD eyes which undergo geographic atrophy (Bird, et al., JAMA Ophthalmol 132, 338-345 (2014)). Thus, in AMD patients, integration of a transplanted iRPE-patch can use “young” iRPE cells secreting metalloproteases that can modify “aged” Bruch's membrane (Greene, et al., Journal of Ocular Pharmacology and Therapeutics: The Official Journal of the Association for Ocular Pharmacology and Therapeutics 33, 132-140 (2017).). In comparison to the PLGA-iRPE-patch and transwell-iRPE-patch, an RPE cell suspension only demonstrated occasional integration as suggested previously (Weisz et al., Retina (Philadelphia, Pa.) 19, 540-545 (1999)) (FIGS. 3C, F).

While the RCS rat model and the pig laser induced RPE injury model do not fully recapitulate AMD pathophysiology, these models provide critical insight in the survival, integration, and potential efficacy of AMD patient-derived iRPE cells (FIGS. 3-6). Retinal function recovery with RPE suspension and iRPE—patches was different in rodents and pigs. A similar degree of visual function rescue was observed from both cell suspension (100,000 cells) and the iPSC-RPE-patch (2,500 cells). Cells in suspension do not form an intact polarized monolayer in the back of the rat eye, but rather behave as a chemical bioreactor that isotopically secretes neurotrophic factors. Unlike a cell suspension, the entire RPE-patch is a polarized cell monolayer that integrates into the rat eye and simultaneously serves the needs of the overlying photoreceptors while maintaining the integrity of its interface with the choroid. In pigs where an equal number of cells were transplanted in a 4×2 mm patch v/s 100,000 cell suspension, significantly higher protection of photoreceptors was seen with the patch. Furthermore, clinical-grade iRPE-patch from different AMD patients behaved similar (FIG. 5) suggesting a reproducible manufacturing process.

Thus, provided herein are a more robust clinical-grade manufacturing process and a RPE-patch on a biodegradable scaffold for better integration and functionality.

### Example 6

CRISPR Mediated Gene Correction in Albinism iPSC

Patient iPSCs were obtained that contained a heterozygous c.593C>T(p.P198L) in OCA2 gene resulting in loss of activity of this gene. This mutation was corrected using CRISPR/Cas9 technology. Guide-RNAs were designed and obtained from commercial sources to the proximity of exact genomic location of c.593C>T mutation in OCA2 gene. A donor plasmid that contained wild type sequence at this location was also obtained commercially. The donor plasmid also contained a puramycin selection cassette and green fluorescent protein (GFP) encoding cDNA. The guide RNAs, wildtype donor plasmid, and CAS9 protein encoding plasmid were all transfected in iPSCs using lipofectamine reagents (ThermoFisher). Puramycin resistant iPSCs colonies were selected and expanded. Selected colonies were further purified by flow cytometry for GFP. Mutation correction was confirmed by sequencing. The corrected iPSCs were then differentiated into the RPE patch using the methods disclosed above.

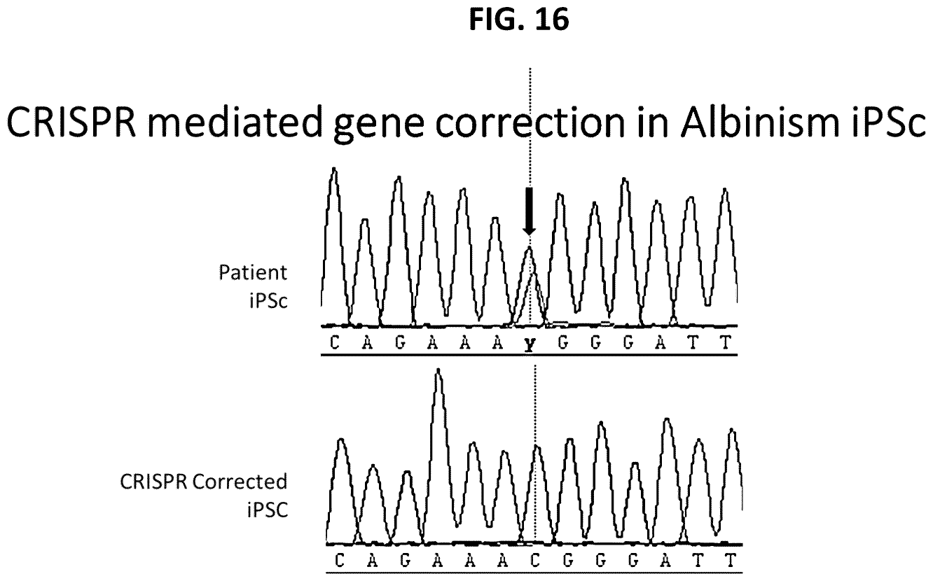

In FIG. 16, the top panel shows the sequence of region around the mutation area (SEQ ID NO: 1, wherein X is C or T). Note, two peaks, one for cytosine and one for thymidine showed the patient was heterozygous at this location. The bottom panel shows after gene correction, there is only one peak for nucleotide cytosine (SEQ ID NO: 1, wherein X is C).

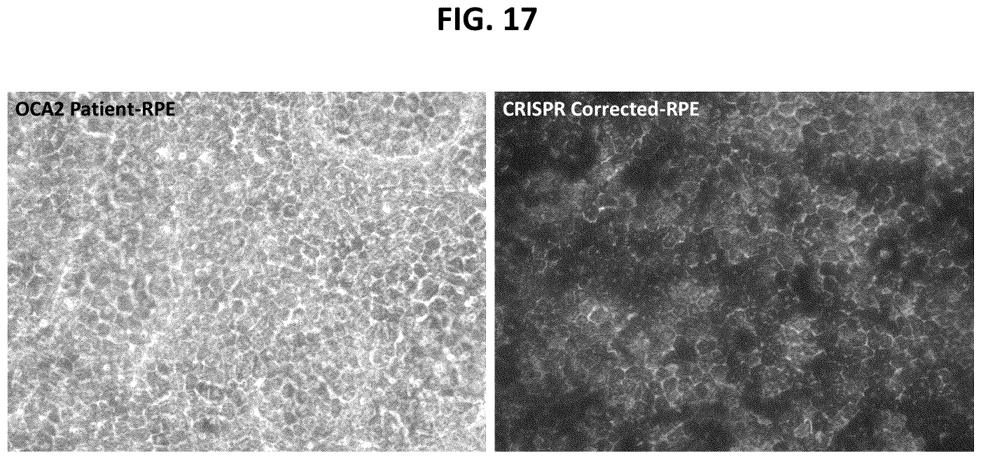

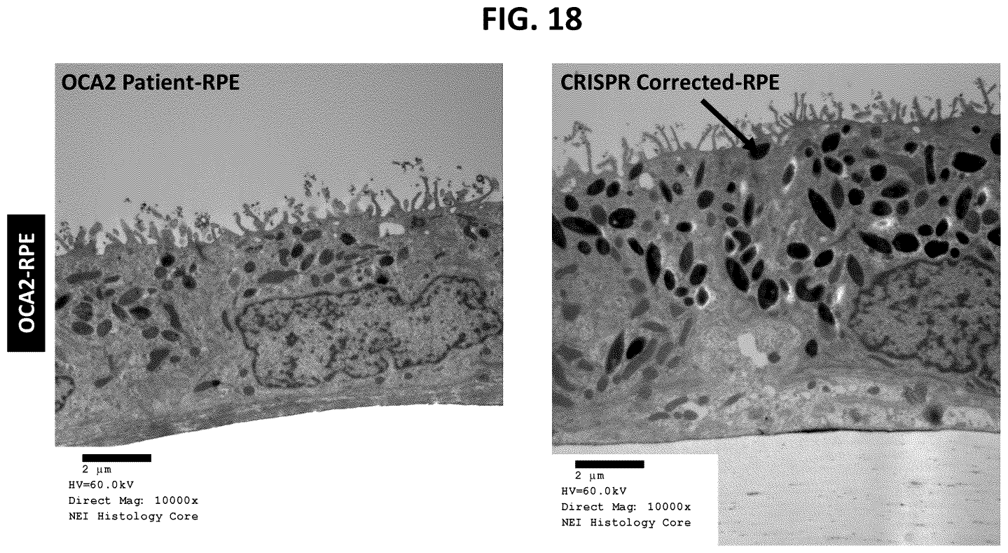

As shown in FIG. 17, when RPE cells from the patient iPSC line were differentiated into an RPE-patch, these cells stay non-pigmented because they the lack activity of the OCA2 protein product. In contrast, when CRISPR-corrected iPSCs are differentiated into an RPE-patch, they were pigmented. Thus, the RPE patch technology works well with genetically engineered cells and can be used to treat patients with monogenic diseases. FIG. 18 also shows OCA2 patient RPE, and CRISPR corrected RPE.

It will be apparent that the precise details of the methods or compositions described may be varied or modified without departing from the spirit of the described invention. We claim all such modifications and variations that fall within the scope and spirit of the claims below.

1. A tissue replacement implant, comprising:
polarized retinal pigment epithelial cells on a poly(lactic-co-glycolic acid) (PLGA) scaffold, wherein the PLGA scaffold is 20-30 microns in thickness, has a DL-lactide/glycotide ratio of about 1:1, an average pore size of less than about 1 micron, and a fiber diameter of about 150 to about 650 nm.

2. The tissue replacement implant of claim 1, wherein the polarized retinal pigment epithelial cells are human.

3. The tissue replacement implant of claim 1, wherein the polarized retinal pigment epithelial cells are produced from induced pluripotent stem cells or ES cells.

4. A method of treating a subject with a retinal degenerative disease, retinal or retinal pigment epithelium dysfunction, retinal degradation, retinal damage, physical injury to the retina, or loss of retinal pigment epithelium, comprising locally administering to the eye of the subject the tissue replacement implant of claim 1, thereby treating the subject.

5. The method of claim 4, wherein the retina degenerative disease is Stargardt's macular dystrophy, retinitis pigmentosa, age related macular degeneration, glaucoma, diabetic retinopathy, Lebers congenital amaurosis, late-onset retinal degeneration, hereditary macular or retinal degeneration, Best disease, Sorsby's fundus dystrophy, retinal detachment, gyrate atrophy, traumatic eye injury, or choroideremia, pattern dystrophy.


SEQUENCE LISTING (2 sequences)

SEQ ID NO: 1 - DNA, 13 bp, Homo sapiens
  source 1..13: mol_type = genomic DNA
cagaaaygggatt

SEQ ID NO: 2 - DNA, 13 bp, Homo sapiens
  source 1..13: mol_type = genomic DNA
cagaaacgggatt